(19) United States
(12) Patent Application Publication
(10) Pub. No.: US 2020/0216825 A1
(43) Pub. Date: Jul. 9, 2020

(54) CAS12a MUTANT GENES AND POLYPEPTIDES ENCODED BY SAME

(71) Applicant: Integrated DNA Technologies, Inc., Coralville, IA (US)

(72) Inventors: Christopher Anthony Vakulskas, North Liberty, IA (US); Nicole Mary Bode, Oxford, IA (US); Michael Allen Collingwood, North Liberty, IA (US); Sarah Beaudoin, Iowa City, IA (US)

(21) Appl. No.: 16/735,841

(22) Filed: Jan. 7, 2020

Related U.S. Application Data
(60) Provisional application No. 62/789,571, filed on Jan. 8, 2019.

(51) Int. Cl. C12N 9/22

(52) U.S. Cl. CPC C12N 9/22; C12N 2310/20

(57) ABSTRACT

This invention pertains to mutant Cas12a nucleic acids and proteins for use in CRISPR/Cas2a endonuclease systems, and their methods of use. IN particular, the invention pertains to an isolated mutant Cas12a protein, wherein the isolated mutant Cas12a protein is active in a CRISPR/Cas12a endonuclease system. The invention also includes isolated nucleic acids encoding mutant Cas12a proteins, ribonucleoprotein complexes and CRISPR/Cas12a endonuclease systems having mutant Cast12a proteins.

### CROSS-REFERENCE TO RELATED APPLICATIONS

This application claims the benefit of U.S. Provisional Application No. 62/789,571, filed Jan. 8, 2019 the disclosure of which is hereby incorporated by reference in its entirety.

### FIELD OF THE INVENTION

This invention pertains to Cas12a based CRISPR genes, polypeptides encoded by the same, mammalian cell lines that stably express Cas12, crRNAs and the use of these materials in compositions of CRISPR-Cas12a systems and methods.

### BACKGROUND OF THE INVENTION

Cas12a (previously named Cpf1) is a class 2/type V CRISPR RNA-guided endonuclease. (Zetsche, B et al., (2015) Cas12a is a single RNA-guided endonuclease of a class 2 CRISPR-Cas system. Cell 163:1-13). Cas12a is an effective nuclease used for genome editing and is an alternative to the Cas9 enzyme. Cas12a is a 1300 amino acid protein and is slightly smaller than Cas9 from S. pyogenes. The Cas12 system does not utilize a separate tracrRNA, and only requires a single short crRNA of 40-45 nucleotides in length that both specifies target DNA sequence and directs binding of the RNA to the Cas12a nuclease. (Hur, J. K., et al. (2016) Targeted mutagenesis in mice by electroporation of Cas12a ribonucleoproteins. Nature Biotechnology, 34:807-808). The PAM recognition sequence of Cas12a is TTTV which allows for expanded coverage in Thymidine rich areas of the genome that Cas9 cannot access.

Cleavage by Cas12a results in a staggered double-stranded break in the DNA with 4-5 nucleotide overhangs, which leaves staggered ends distal to the PAM site (Gao, P. et al., (2016) Type V CRISPR-Cas Cas12a endonuclease employs a unique mechanism for crRNA-mediated target DNA recognition. Cell Research 26:901-913. These double stranded breaks can then be repaired via non-homologous end joining (NHEJ) which often leads to mutations or insertions/deletions at the cut site or site or homology directed repair (HDR) which can generate precise editing events. Furthermore, when Cas12a cleaves, it does so further away from PAM than Cas9, which is also further away from the target site. As a result, the protospacer, and especially the seed sequence of the protospacer, are less likely to be edited, thereby leaving open the potential for a second round of cleavage if the desired repair event doesn't occur the first time.

Proteins are often unstable outside of living cells, which can make using them as therapeutics difficult. Disulfide bonds in proteins have been shown to be important in both stability and activity of the protein. When oxidized, cysteine residues can form disulfide bonds. These disulfide bonds are formed when the free thiol groups on cysteine residues are oxidized. The only naturally occurring amino acids containing sulfur are cysteine or methionine. However, the sulfur in methionine is not free and therefore cannot form a disulfide linkage.

Due to the free thiol the amino acid cysteine can be involved in formation of intra- or inter-molecular disulfide bonds or may remain as free thiol. In certain proteins, cysteine residues help stabilize or maintain enzymatic function (Trivedi M V, et al. The role of thiols and disulfides in protein chemical and physical stability. Current protein and peptide science. 2009: 100:614-625). In a peptide or protein, the presence of disulfide bridges provides structural rigidity and proper folding is necessary to form native disulfide bonds and to preserve function of the protein. However, when purifying protein, the free thiol groups on cysteine may form unnatural disulfide bonds which may negatively impact the protein structure, function, and stability. Cas12a has eight cysteine residues and the potential for disulfide bond formation is high, which may be problematic for the isolation of a properly folded protein during purification and/or the possibility to decrease long term storage stability.

The large number of cysteine residues increases the likelihood of unnatural disulfide bridging during protein isolation and the unnatural disulfide bridging may impact the protein function and long term storage stability of the purified protein. Unnatural bridging can lead to improper folding of the Cas12a protein and negatively impact the protein's effectivity and may decrease the long term stability of the isolated protein. To reduce the likelihood of unnatural disulfide bridging the proteins of interest may be isolated in non-oxidizing systems. However, this increases costs of purification and also makes the purification and isolation more difficult.

Optionally following isolation and purification the protein of interest may be treated with reducing agents such as dithiothreitol or mercaptoethanol. Dithiothreitol (DTT) or mercaptoethanol can break the disulfide bridge yielding free sulfhydryl groups. However, treatment with reducing agents such as DTT or mercaptoethanol may not always reduce all unnatural disulfide bridges, complicates isolation and purification schemes, may negatively affect the protein structure and may negatively impact long term storage of the protein.

There is therefore a need to modify the Cas12a protein to both aid in purification and increase the long term storage stability while still performing the intended purpose of RNA-targeted cleavage. Furthermore, multiple cysteines in the native Cas12a prevents the site specific introduction or covalent linkage of additional functional groups and site specific immobilization of the polypeptide. The methods and compositions of the invention described herein provide modified Cas12a proteins. These and other advantages of the invention, as well as additional inventive features, will be apparent from the description of the invention provided herein.

### BRIEF SUMMARY OF THE INVENTION

This invention pertains to Cas12a CRISPR genes and mutants, polypeptides encoded by the same, mammalian cell lines that stably express Cas12a and their use in compositions of CIRSPR-Cas12a systems and methods. Examples are shown employing the Cpf1 systems from Acidaminococcus sp. BV3L6 however this is not intended to limit scope, which extends to Cas12a homologs or orthologs isolated from other species.

The selective substitution of cysteine with other non-sulfur containing amino acids aids in purification of the Cas12a protein because the purification does not need to occur in a non-oxidizing system. Additionally, when the purified enzyme is isolated there is no need to store with reducing agents such is dithiothreitol or mercaptoethanol. Furthermore, it is possible that reducing agents will not properly reduce all the unnatural disulfide linkages thereby affecting the proteins structure, effectiveness, and long term storage stability.

In one embodiment mutant Cas12a (also known as Cpf1) enzymes are designed by substituting cysteine amino acid residues with non-thiol containing amino acid residues. For example, cysteine may be selectively substituted with other non-thiol containing amino acids. In selectively substituting cysteine residues one or more of the cysteine residues may be substituted with another amino acid. In selectively substituting cysteine residues all but one cysteine residue are substituted with another amino acid. In another embodiment cysteine is selectively substituted with other similar size and non-thiol containing amino acids. In a further embodiment cysteine is selectively substituted with glycine. In yet another embodiment cysteine is selectively substituted with polar amino acids such as serine or threonine. In another embodiment cysteine is selectively substituted with serine.

In one embodiment all cysteine residues are substituted with another amino acid. In a further embodiment all cysteine residues are substituted with other non-thiol containing amino acids. In another embodiment all cysteine residues are selectively substituted with other similar size and non-thiol containing amino acids. In a further embodiment all cysteine residues are selectively substituted with glycine. In yet another embodiment all cysteine residues are selectively substituted with polar amino acids such as serine or threonine. In another embodiment all cysteine residues are substituted with serine.

In another embodiment all but one cysteine residue are substituted with another amino acid. In a further embodiment all but one cysteine residue are substituted with other non-thiol containing amino acids. In another embodiment all but one cysteine residue are substituted with other similar size and non-thiol containing amino acids. In a further embodiment all but one cysteine residue are substituted with glycine. In a further embodiment all but one cysteine residue are selectively substituted with polar amino acids such as serine or threonine. In yet another embodiment all but one cysteine residue are selectively substituted with serine.

By selectively replacing the cysteine residues one can selectively label the protein of interest. The selective labels, or modifications, can be covalently attached to the remaining cysteine residue, remaining cysteine residues, or the selectively introduced cysteine residues. Alternatively, all of the cysteine residues are substituted with another amino acid. Cysteine may be reintroduced at selected locations and the modifications can be covalently attached at the reintroduced cysteine residue.

In a further embodiment cysteine may be selectively reintroduced into the polypeptide. In some embodiments cysteine may be selectively introduced at the C-terminal or N-terminal ends of the protein. In other embodiments cysteine may be selectively introduced to internal sites of the polypeptide. By selectively introducing cysteine to one or both terminal ends, or to internal sites, one can then selectively modify the protein and enable a range of biological and biophysical studies. Various modifications may be conjugated or covalently linked to the cysteine modified polypeptide. Sulfyrdryls that exist in the side chain of cysteine are commonly targeted for bioconjugation. Typically, only free or reduced sulfhydryl groups are available for reaction with thiol-reactive compounds. Sulfhydryl-reactive chemical groups include, but are not limited to, haloacetyls, maleimides, aziridines, acryloyls, arylating agents, vinylsulfones, pyridyl disulfides, TNB-thiols and disulfide reducing agents. Most of these groups conjugate to sulfhydryls by either alkylation (usually the formation of a thioether bond) or disulfide exchange (formation of a disulfide bond). Bioconjugation includes crosslinking, immobilization, surface modification and labeling of biomolecules. One skilled in the art would appreciate these various crosslinking technologies, as well as how to make and use proteins that include them.

In another embodiment affinity ligands are immobilized through the thiol group of the cysteine residues. Because amines occur at many positions on a protein's surface, it is usually difficult to predict where a coupling reaction will occur. Removing all cysteine residues and selectively reintroducing a cysteine or cysteine residues enables more selective immobilization of proteins and peptides. Cysteine residues may be added to terminal ends of the polypeptide or at selected internal locations. This selective placement ensures that every peptide molecule will be oriented on the support in the same way after immobilization. By selectively placing cysteine residues on the polypeptide it is possible to immobilize the polypeptide by covalently linkage through disulfide bridging. Various immobilization supports include, for example, but not limited to, are maleimide-activated supports, iodoacetyl-activated supports, or pyridyl disulfide supports.

In another embodiment mutant Cas12a was designed where each cysteine residue of the wild-type Cas12a was individually changed to a serine residue. Serine is an amino acid with a structurally-similar functional group to cysteine, but serine does not facilitate disulfide bridging. In this embodiment eight individual Cas12a mutants were designed in which each individual cysteine was substituted serine. However, it should be understood that substitutions with other amino acid residues are contemplated by the present invention. Individual cysteine substitutions were made at C65S, C205S, C334S, C379S, C608S, C674S, C1025S, and C1248S.

In other embodiments each of the individual cysteine to serine Cas12a mutants were tested for enzymatic activity in a bacterial cleavage system to determine the cleavage efficiency of the Cas12a substation mutants.

In another aspect the Cas12a mutants were subsequently tested for gene editing efficiency in human cells when the Cas12a mutants were delivered by plasmids in a tissue culture setting.

In another embodiment multi-substitution mutants were designed. Multi-substitution mutant Cas12a proteins include mutations in the WT-Cas12a introduced to at least two cysteine residues. It is contemplated that any specific multi-substitution mutants may be designed in which different cysteine residues and different combinations of cysteine residues are substituted. Multi substitution mutants may consist of substituting one or a combination of any of the eight cysteine residues present in the Cas12a mutant. For example, multi-substitution mutants may comprise the substitution of cysteine residues at amino residue C65, C205, C334, C379, C608, C674, C1025, and C1248 and combination of any substitutions at the cysteine residues. For example, multi-substitution mutants may comprise substitution of cysteine at C205S, C379S, C674S, and C1248S. In another example a multi-substitution mutant comprises substitutions at C65S, C205S, C334S, C379S, C674S and C1248S.

In yet another embodiment Cas12a mutants were tested for editing efficiency in human cells. In this embodiment one Cas12a mutant was generated by combining four of the eight cysteine to serine mutants into a single multi-substitution Cas12a mutant. This mutant comprised cysteine to serine substitutions at C205S, C379S, C674S, and C1248S. This multiple mutant was purified and tested for cleavage efficiency in human cells.

In another embodiment the Cas12a mutants were tested for editing efficiency in human cells. In this embodiment one Cas12a mutant was generated by combining 6 of the eight cysteine to serine mutants into a single Cas12a mutant. This mutant comprised C65S, C205S, C334S, C379S, C674S, and C1248S substitutions.

In another embodiment the Cas12a mutants were tested for editing efficiency in human cells when the purified Cas12a mutant protein was directly delivered into human cells by forming a RNP complex.

The present invention identifies amino acid positions in the (Acidaminococcus sp. BV3L6) AsCas12a gene that can be mutated from cysteine to alternative amino acids. The present invention identifies amino acid positions in the (Acidaminococcus sp. BV3L6) AsCas12a gene that can be mutated from cysteine to serine to reduce the likelihood of unnatural disulfide bridging. Surprisingly, combined mutations also increased the genome editing efficiency achieved with plasmid delivery of CRISPR reagents when compared to the unaltered wild-type Cas12a protein.

### BRIEF DESCRIPTION OF THE DRAWINGS

FIG. 1 shows the editing efficiency of the Cas12a mutants as compared to wild-type Cas12a when the protein is expressed from a plasmid.

FIG. 2 shows the editing efficiency of the Cas12a mutants as compared to wild-type Cas12a when the protein is delivered by RNP.

### DETAILED DESCRIPTION OF THE INVENTION

The methods and compositions of the invention described herein provide mutant Cas12a nucleic acids and polypeptides for use in a CRISPR/Cas12a system. The present invention describes mutant Cas12a protein with cysteine substitutions. In another embodiment the cysteine residues of the Cas12a protein are individually substituted with serine. In additional embodiments the Cas12a protein is modified to include multiple cysteine substitutions.

Cas12a provides a useful complement to Cas9 by expanding the range of PAM sequences that can be targeted from GC-Rich areas (Cas9) to AT-rich areas of the genome (Cas12a), thereby expanding the range of sequences that can be modified using CRISPR genome engineering methods. In addition to having a T-rich PAM site, another advantage of the Cas12a system as compared with Cas9 is the use of a single short RNA molecule.

Proteins are often unstable outside of living cells, which can make using them as therapeutics difficult. Disulfide bonds in proteins have been shown to be important in both stability and activity of the protein. These bonds are formed when the thiol groups on cysteine residues are oxidized. The amino acid cysteine can be involved in formation of intra- or inter-molecular disulfide bonds or may be present as free thiols. In certain proteins, cysteine residues are critical for enzymatic function (Trivedi, M. V., et al (2009) The role of thiols and disulfides in protein chemical and physical stability. Current protein & peptide science. 10(6):614-625). In a peptide or protein, the presence of disulfide bridges provides structural rigidity and proper folding is necessary to form native disulfide bonds and to preserve function of the protein. Cas12a has eight cysteine residues, the potential for disulfide bond formation is high, and this could be problematic for isolation of properly folded protein during purification and/or decrease long term storage stability.

By selectively replacing the cysteine residues with non-thiol containing amino acids the potential for unnatural disulfide bond formation can be reduced. In one embodiment the cysteine residues can be substituted with any non-thiol containing amino acid. In another embodiment the cysteine residues can be replaced with non-thiol containing yet similar sized amino acids, e.g., threonine or serine. In another embodiment the cysteine residues can be replaced, individual or collectively, with serine.

Changing the cysteines to serine, in certain locations, improved the cleavage activity of Cas12a when compared to WT Cas12a. This improved efficiency makes this a more effective endonuclease to be used in CRISPR applications and can replace the current Cas12a protein being used. Furthermore, changing the cysteine to serine, in certain locations, may improve the long term storage stability of the isolated and purified polypeptide.

In a first aspect mutant Cas12a proteins were developed by changing the cysteine residues of the wild-type Cas12a enzyme to serine. Serine is an amino acid with a structurally-similar functional group to cysteine but it does not facilitate disulfide bridging. Cas12a mutants were developed to provide a more stable Cas12a protein and to aid in purification and isolation of the protein as well as to increase the long term storage stability of the protein. Preferred single mutant Cas12a proteins include substitution mutations in the WT-Cas12a introduced at one of the following positions: C65, C205, C334, C379, C608, C674, C1025, and C1248. Exemplary single mutant Cas12a proteins include the following specific mutations introduced into the WT-Cas12a: C65S, C205S, C334S, C379S, C608S, C674S, C1025S, and C1248S. Exemplary single mutant Cas12a proteins include at least one member selected from the group consisting of SEQ ID Nos: 3-12. Additional substitution mutations can be included in the amino acid backgrounds of the single mutant CAs12a protein amino acid sequences, provided that the resultant Cas12 protein is active as a CRISPR/Cas12a endonuclease system, wherein the resultant CRISPR/Cas12a endonuclease system displays maintained on-target editing activity relative to a wild-type CRISPR/Cas12a endonuclease system.

Preferred multi-substitution mutant Cas12a proteins include mutations in the WT-Cas12a introduced to at least two of the following positions: C65S, C205S, C334S, C379S, C608S, C674S, C1025S, and C1248S. Exemplary multi-substitution mutant Cas12a proteins include mutations in the WT-Cas12a selected from the following amino acid mutations: C65S, C205S, C334S, C379S, C608S, C674S, C1025S, and C1248S. Exemplary multi substitution mutant Cas12a proteins include at least one member selected from the group consisting of SEQ ID Nos: 13-14. Additional substitution mutations can be included in the amino acid backgrounds of the multi-substitution mutant Cas12a protein amino acid sequences, provided that the resultant mutant Cas12a protein is active as a CRISPR/Cas12a endonuclease system, wherein the resultant CRISPR/Cas endonuclease system displays maintained on-target editing activity relative to a wild-type CRISPR/Cas12a endonuclease system

In another aspect, an isolated ribonucleoprotein complex is provided. The RNP includes mutant Cas12a protein and a gRNA complex. In one respect, the gRNA includes a crRNA and a tracrRNA in stoichiometric (1:1) ratio. In a second respect the crRNA includes an Alt-R® crRNA (Integrated DNA Technologies, Inc. (Coralville, Iowa, (US)) directed against a specific editing target site for a given locus and the tracrRNA includes Alt-R® tracrRNA (Integrated DNA Technologies, Inc. (Coralville, Iowa (US)). In another respect the gRNA includes a sgRNA. Preferred mutant Cas9 proteins include those as described above.

In an aspect, an isolated nucleic acid encoding a mutant Cas12a protein is provided. Preferred isolated nucleic acids encode mutant Cas12a proteins as described above. Exemplary isolated nucleic acids encoding mutant Cas12a proteins can be readily generated from a nucleic acid encoding the wild-type Cas12a protein using recombinant DNA procedures or chemical synthesis methods. Preferred nucleic acids for this purpose include those optimized for expression of the Cas12a proteins in bacteria, (e.g., E. coli.) or mammalian (e.g., human) cells. Exemplary codon-optimized nucleic acids for expressing WT-Cas12a in E. coli and human cells includes SEQ ID NO. 1. Moreover, the present invention contemplates fusion proteins of WT-Cas12a and mutant Cas12a, wherein the coding sequences of WT-Cas12a and mutant Cas12a are fused to amino acid sequences encoding for nuclear localization (“NLS”) of the fusion protein in eukaryotic cells or amino acid sequences to facilitate purification of the proteins.

In one respect, the isolated nucleic acid includes mRNA encoding one of the aforementioned mutant Cas12a proteins. In a second respect, the isolated nucleic acid includes DNA encoding a gene for one of the aforementioned mutant Cas12a proteins. A preferred DNA includes a vector that encodes a gene encoding for a mutant Cas12a protein. Such delivery methods include plasmid and various viral delivery vectors as are well known to those with skill in the art. The mutant Cas12a protein can also be stably transformed into cells using suitable expression vectors to produce a cell line that constitutively or inducibly expresses the mutant Cas12a. The aforementioned methods can also be applied to embryos to product progeny animals that constitutively or inducibly expresses the mutant Cas12a.

In another aspect a CRISPR/Cas12a endonuclease system is provided. The CRISPR/Cas12a endonuclease system includes a mutant Cas12a protein. Preferred mutant Cas12a proteins include those as described above. In one respect, the CRISPR/Cas12a endonuclease system is encoded by a DNA expression vector. In one embodiment, the DNA expression vector is a plasmid-borne vector. In a second embodiment, the DNA expression vector is selected from a bacterial expression vector and a eukaryotic expression vector. In third respect, the CRISPR/Cas12a endonuclease system comprises a ribonucleoprotein complex comprising a mutant Cas12a protein and a gRNA complex. In one respect, the gRNA includes a crRNA and a tracrRNA in stoichiometric (1:1) ratio. In a second respect the crRNA includes an Alt-R® crRNA (Integrated DNA Technologies, Inc. (Coralville, Iowa (US)) directed against a specific editing target site for a given locus and the tracrRNA includes Alt-R® tracrRNA (Integrated DNA Technologies, Inc. (Coralville, Iowa (US)). In another respect the gRNA includes a sgRNA.

In a fifth aspect, a method of performing gene editing having reduced off-target editing activity and/or increased on-target editing activity is provided. The method includes the step of contacting a candidate editing target site locus with an active CRISPR/Cas endonuclease system having a mutant Cas12a protein. In one respect, the method includes single mutant Cas12a proteins having mutations in the WT-Cas12a introduced at one of the following positions: C65, C205, C334, C379, C608, C674, C1025, and C1248. Exemplary single mutant Cas12a proteins include the following specific mutations introduced into the WT-Cas12a: C65S, C205S, C334S, C379S, C608S, C674S, C1025S, and C1248S. Exemplary single mutant Cas12a proteins include at least one member selected from the group consisting of SEQ ID Nos: 3-12. Additional substitution mutations can be included in the amino acid backgrounds of the single mutant CAs12a protein amino acid sequences, provided that the resultant Cas12 protein is active as a CRISPR/Cas12a endonuclease system, wherein the resultant CRISPR/Cas12a endonuclease system displays maintained on-target editing activity relative to a wild-type CRISPR/Cas12a endonuclease system.

In another respect the methods include a multi-substitution mutant Cas12 protein having mutations in the WT-Cas12a introduced to at least two of the following positions: C65S, C205S, C334S, C379S, C608S, C674S, C1025S, and C1248S. Exemplary multi-substitution mutant Cas12a proteins include mutations in the WT-Cas12a selected from the following amino acid mutations: C65S, C205S, C334S, C379S, C608S, C674S, C1025S, and C1248S. Exemplary multi substitution mutant Cas12a proteins include at least one member selected from the group consisting of SEQ ID Nos: 13-14. Additional substitution mutations can be included in the amino acid backgrounds of the multi-substitution mutant Cas12a protein amino acid sequences, provided that the resultant mutant Cas12a protein is active as a CRISPR/Cas12a endonuclease system, wherein the resultant CRISPR/Cas endonuclease system displays maintained on-target editing activity relative to a wild-type CRISPR/Cas12a endonuclease system

### Example 1

### DNA and Amino Acid Sequences of Wild Type and Mutant Cas12a Proteins.

The list below shows different wild type (WT) and mutant Cas12a nucleases described in present invention. It will be appreciated by one with skill in the art that many different DNA sequences can encode/express the same amino acid (AA) sequence since in many cases more than one codon can encode the same amino acid. The DNA sequences shown below only serve as example and other DNA sequences that encode the same protein (e.g., same amino acid sequence) are contemplated. It is further appreciated that additional features, elements or tags may be added to said sequences, such as NLS domains and the like. Examples are shown for WT Cas12a (Cpf1) and mutant C65S Cas12a, mutant C205S Cas12a, mutant C334S Cas12a, mutant C379S Cas12a, mutant C608S Cas12a, mutant 674S Cas12a, mutant C1025S Cas12a, mutant C1248S Cas12a, multi-combination mutant C205S, C379S, C674S, C1248S Cas12a mutant, multi-combination mutant C65S, C205S, C334S, C379S, C674S, C1248 S Cas12a mutant and amino acid sequences. For Cas12a mutants only the amino acid sequences are provided, but it is contemplated that NLS domains and His-tag domains may be added to facilitate use in producing recombinant proteins for use in mammalian cells.

WT Cas12a DNA sequence, codon optimized for expression
in Human
SEQ ID NO. 1
ATGACCCAGTTCGAGGGCTTCACCAACCTGTACCAGGTGTCCAAGACCCTGA
GATTCGAGCTGATCCCCCAGGGCAAGACACTGAAGCACATCCAGGAACAGG
GCTTCATCGAAGAGGACAAGGCCCGGAACGACCACTACAAAGAGCTGAAGC
CCATCATCGACCGGATCTACAAGACCTACGCCGACCAGTGCCTGCAGCTGGT
GCAGCTGGACTGGGAGAATCTGAGCGCCGCCATCGACAGCTACCGGAAAGA
GAAAACCGAGGAAACCCGGAACGCCCTGATCGAGGAACAGGCCACCTACAG
AAACGCCATCCACGACTACTTCATCGGCCGGACCGACAACCTGACCGACGC
CATCAACAAGCGGCACGCCGAGATCTATAAGGGCCTGTTCAAGGCCGAGCT
GTTCAACGGCAAGGTGCTGAAGCAGCTGGGCACCGTGACCACCACCGAGCA
CGAAAACGCCCTGCTGCGGAGCTTCGACAAGTTCACCACCTACTTCAGCGGC
TTCTACGAGAACCGGAAGAACGTGTTCAGCGCCGAGGACATCAGCACCGCC
ATCCCCCACAGAATCGTGCAGGACAACTTCCCCAAGTTCAAAGAGAACTGC
CACATCTTCACCCGGCTGATCACCGCCGTGCCCAGCCTGAGAGAACACTTCG
AGAACGTGAAGAAGGCCATCGGCATCTTCGTGTCCACCAGCATCGAGGAAG
TGTTCAGCTTCCCATTCTACAACCAGCTGCTGACCCAGACCCAGATCGACCT
GTATAATCAGCTGCTGGGCGGCATCAGCAGAGAGGCCGGCACCGAGAAGAT
CAAGGGCCTGAACGAAGTGCTGAACCTGGCCATCCAGAAGAACGACGAGAC
AGCCCACATCATTGCCAGCCTGCCCCACCGGTTCATCCCTCTGTTCAAGCAG
ATCCTGAGCGACAGAAACACCCTGAGCTTCATCCTGGAAGAGTTCAAGTCCG
ATGAGGAAGTGATCCAGAGCTTCTGCAAGTATAAGACCCTGCTGAGGAACG
AGAATGTGCTGGAAACCGCCGAGGCCCTGTTCAATGAGCTGAACAGCATCG
ACCTGACCCACATCTTTATCAGCCACAAGAAGCTGGAAACAATCAGCAGCG
CCCTGTGCGACCACTGGGACACACTGCGGAATGCCCTGTACGAGCGGCGGA
TCTCTGAGCTGACCGGCAAGATCACCAAGAGCGCCAAAGAAAAGGTGCAGC
GGAGCCTGAAGCACGAGGATATCAACCTGCAGGAAATCATCAGCGCCGCTG
GCAAAGAACTGAGCGAGGCCTTTAAGCAGAAAACCAGCGAGATCCTGTCCC
ACGCCCACGCCGCACTGGATCAGCCTCTGCCTACCACCCTGAAGAAGCAGG
AAGAGAAAGAGATCCTGAAGTCCCAGCTGGACAGCCTGCTGGGCCTGTACC
ATCTGCTGGATTGGTTCGCCGTGGACGAGAGCAACGAGGTGGACCCCGAGT
TCTCCGCCAGACTGACAGGCATCAAACTGGAAATGGAACCCAGCCTGTCCTT
CTACAACAAGGCCAGAAACTACGCCACCAAGAAACCCTACAGCGTGGAAAA
GTTTAAGCTGAACTTCCAGATGCCCACCCTGGCCAGCGGCTGGGACGTGAAC
AAAGAGAAGAACAACGGCGCCATCCTGTTCGTGAAGAACGGACTGTACTAC
CTGGGCATCATGCCTAAGCAGAAGGGCAGATACAAGGCCCTGTCCTTTGAG
CCCACCGAAAAGACCAGCGAGGGCTTTGACAAGATGTACTACGATTACTTCC
CCGACGCCGCCAAGATGATCCCCAAGTGCAGCACCCAGCTGAAGGCCGTGA
CCGCCCACTTTCAGACCCACACCACCCCCATCCTGCTGAGCAACAACTTCAT
CGAGCCCCTGGAAATCACCAAAGAGATCTACGACCTGAACAACCCCGAGAA
AGAGCCCAAGAAGTTCCAGACCGCCTACGCCAAGAAAACCGGCGACCAGAA
GGGCTACCGCGAGGCTCTGTGCAAGTGGATCGACTTTACCCGGGACTTCCTG
AGCAAGTACACCAAGACCACCTCCATCGATCTGAGCAGCCTGCGGCCCAGC
TCCCAGTACAAGGATCTGGGCGAGTACTACGCCGAGCTGAACCCTCTGCTGT
ACCACATCAGCTTCCAGCGGATCGCCGAAAAAGAAATCATGGACGCCGTGG
AAACCGGCAAGCTGTACCTGTTCCAGATCTATAACAAGGACTTCGCCAAGG
GCCACCACGGCAAGCCCAATCTGCACACCCTGTACTGGACCGGCCTGTTTAG
CCCCGAGAATCTGGCCAAGACCAGCATCAAGCTGAACGGCCAGGCCGAACT
GTTTTACCGGCCCAAGAGCCGGATGAAGCGGATGGCCCATAGACTGGGCGA
GAAGATGCTGAACAAGAAACTGAAGGACCAGAAAACCCCTATCCCCGACAC
ACTGTATCAGGAACTGTACGACTACGTGAACCACCGGCTGAGCCACGACCT
GTCCGACGAAGCTAGAGCACTGCTGCCCAACGTGATCACAAAAGAGGTGTC
CCACGAGATCATCAAGGACCGGCGGTTTACCTCCGATAAGTTCTTCTTCCAC
GTGCCCATCACCCTGAACTACCAGGCCGCCAACAGCCCCAGCAAGTTCAACC
AGAGAGTGAACGCCTACCTGAAAGAGCACCCCGAGACACCCATCATTGGCA
TCGACAGAGGCGAGCGGAACCTGATCTACATCACCGTGATCGACAGCACAG
GCAAAATCCTGGAACAGAGAAGCCTGAACACCATCCAGCAGTTCGACTACC
AGAAGAAACTGGACAACCGGGAAAAAGAACGGGTGGCCGCCAGACAGGCT
TGGAGCGTCGTGGGCACCATTAAGGACCTGAAGCAGGGCTACCTGAGCCAA
GTGATTCACGAGATCGTGGACCTGATGATCCACTATCAGGCTGTGGTGGTGC
TGGAAAACCTGAACTTCGGCTTCAAGAGCAAGCGGACCGGAATCGCCGAGA
AAGCCGTGTACCAGCAGTTTGAGAAAATGCTGATCGACAAGCTGAATTGCCT
GGTGCTGAAAGACTACCCCGCTGAGAAAGTGGGAGGCGTGCTGAATCCCTA
CCAGCTGACCGACCAGTTCACCTCCTTTGCCAAGATGGGAACCCAGAGCGGC
TTCCTGTTCTACGTGCCAGCCCCCTACACCAGCAAGATCGACCCTCTGACCG
GCTTCGTGGACCCCTTCGTGTGGAAAACCATCAAGAACCACGAGTCCCGGA
AGCACTTCCTGGAAGGCTTTGACTTCCTGCACTACGACGTGAAAACAGGCGA
TTTCATCCTGCACTTCAAGATGAATCGGAATCTGTCCTTCCAGAGGGGCCTG
CCCGGCTTCATGCCTGCCTGGGATATCGTGTTCGAGAAGAATGAGACACAGT
TCGACGCCAAGGGAACCCCCTTTATCGCCGGCAAGAGGATCGTGCCTGTGAT
CGAGAACCACAGATTCACCGGCAGATACCGGGACCTGTACCCCGCCAACGA
GCTGATTGCCCTGCTGGAAGAGAAGGGCATCGTGTTCCGGGACGGCAGCAA
CATCCTGCCCAAGCTGCTGGAAAATGACGACAGCCACGCCATCGATACCAT
GGTGGCACTGATCCGCAGCGTGCTGCAGATGCGGAACAGCAATGCCGCCAC
CGGCGAGGACTACATCAATAGCCCAGTGCGGGACCTGAACGGCGTGTGCTT
CGACAGCAGATTCCAGAACCCCGAGTGGCCCATGGATGCCGACGCCAATGG
CGCCTACCACATTGCCCTGAAGGGACAGCTGCTGCTGAACCATCTGAAAGA
GAGCAAAGACCTGAAACTGCAGAACGGCATCTCCAACCAGGACTGGCTGGC
CTATATCCAGGAACTGCGGAAC
WT Cas12a amino acid sequence
SEQ ID NO. 2
MTQFEGFTNLYQVSKTLRFELIPQGKTLKHIQEQGFIEEDKARNDHYKELKPIIDR
IYKTYADQCLQLVQLDWENLSAAIDSYRKEKTEETRNALIEEQATYRNAIHDYFIG
RTDNLTDAINKRHAEIYKGLFKAELENGKVLKQLGTVTTTEHENALLRSEDKFTTY
FSGFYENRKNVFSAEDISTAIPHRIVQDNFPKEKENCHIFTRLITAVPSLREHFEN
VKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFCKY
KTLLRNENVLETAEALFNELNSIDLTHIFISHKKLETISSALCDHWDTLRNALYER
RISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIK
LEMEPSLSFYNKARNYATKKPYSVEKFKLNFQMPTLASGWDVNKEKNNGAILFVKN
GLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREA
LCKWIDFTRDELSKYTKTTSIDLSSLRPSSQYKDLGEYYAELNPLLYHISFQRIAE
KEIMDAVETGKLYLFQIYNKDFAKGHHGKPNLHTLYWTGLFSPENLAKTSIKLNGQ
AELFYRPKSRMKRMAHRLGEKMLNKKLKDQKTPIPDTLYQELYDYVNHRLSHDLSD
EARALLPNVITKEVSHEIIKDRRFTSDKEFFHVPITLNYQAANSPSKFNQRVNAYL
KEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSKRTGIAE
KAVYQQFEKMLIDKLNCLVLKDYPAEKVGGVLNPYQLTDQFTSFAKMGTQSGELFY
VPAPYTSKIDPLTGFVDPFVWKTIKNHESRKHFLEGFDFLHYDVKTGDFILHFKMN
RNLSFQRGLPGFMPAWDIVFEKNETQFDAKGTPFIAGKRIVPVIENHRFTGRYRDL
YPANELIALLEEKGIVERDGSNILPKLLENDDSHAIDTMVALIRSVLQMRNSNAAT
GEDYINSPVRDLNGVCFDSRFQNPEWPMDADANGAYHIALKGQLLLNHLKESKDLK
LQNGISNQDWLAYIQELRN
C65S mutant Cas12a amino acid sequence
SEQ ID NO. 3
MTQFEGFTNLYQVSKTLRFELIPQGKTLKHIQEQGFIEEDKARNDHYKELKPIIDR
IYKTYADQSLQLVQLDWENLSAAIDSYRKEKTEETRNALIEEQATYRNAIHDYFIG
RTDNLTDAINKRHAEIYKGLFKAELFNGKVLKQLGTVTTTEHENALLRSFDKFTTY
FSGEYENRKNVESAEDISTAIPHRIVQDNFPKEKENCHIFTRLITAVPSLREHFEN
VKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFCKY
KTLLRNENVLETAEALFNELNSIDLTHIFISHKKLETISSALCDHWDTLRNALYER
RISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIK
LEMEPSLSFYNKARNYATKKPYSVEKFKLNFQMPTLASGWDVNKEKNNGAILFVKN
GLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREA
LCKWIDETRDELSKYTKTTSIDLSSLRPSSQYKDLGEYYAELNPLLYHISFQRIAE
KEIMDAVETGKLYLFQIYNKDFAKGHHGKPNLHTLYWTGLFSPENLAKTSIKLNGQ
AELFYRPKSRMKRMAHRLGEKMLNKKLKDQKTPIPDTLYQELYDYVNHRLSHDLSD
EARALLPNVITKEVSHEIIKDRRFTSDKEFFHVPITLNYQAANSPSKFNQRVNAYL
KEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNEGEKSKRTGIAE
KAVYQQFEKMLIDKLNCLVLKDYPAEKVGGVLNPYQLTDQFTSFAKMGTQSGELFY
VPAPYTSKIDPLTGFVDPFVWKTIKNHESRKHFLEGFDFLHYDVKTGDFILHFKMN
RNLSFQRGLPGEMPAWDIVFEKNETQFDAKGTPFIAGKRIVPVIENHRFTGRYRDL
YPANELIALLEEKGIVERDGSNILPKLLENDDSHAIDTMVALIRSVLQMRNSNAAT
GEDYINSPVRDLNGVCFDSRFQNPEWPMDADANGAYHIALKGQLLLNHLKESKDLK
LQNGISNQDWLAYIQELRN
C205S mutant Cas12a amino acid sequence
SEQ ID NO. 4
MTQFEGFTNLYQVSKTLRFELIPQGKTLKHIQEQGFIEEDKARNDHYKELKPIIDR
IYKTYADQCLQLVQLDWENLSAAIDSYRKEKTEETRNALIEEQATYRNAIHDYFIG
RTDNLTDAINKRHAEIYKGLFKAELENGKVLKQLGTVTTTEHENALLRSEDKFTTY
FSGFYENRKNVFSAEDISTAIPHRIVQDNFPKFKENSHIFTRLITAVPSLREHFEN
VKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFCKY
KTLLRNENVLETAEALFNELNSIDLTHIFISHKKLETISSALCDHWDTLRNALYER
RISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIK
LEMEPSLSFYNKARNYATKKPYSVEKFKLNFQMPTLASGWDVNKEKNNGAILFVKN
GLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREA
LCKWIDETRDELSKYTKTTSIDLSSLRPSSQYKDLGEYYAELNPLLYHISFQRIAE
KEIMDAVETGKLYLFQIYNKDFAKGHHGKPNLHTLYWTGLFSPENLAKTSIKLNGQ
AELFYRPKSRMKRMAHRLGEKMLNKKLKDQKTPIPDTLYQELYDYVNHRLSHDLSD
EARALLPNVITKEVSHEIIKDRRFTSDKFFFHVPITLNYQAANSPSKFNQRVNAYL
KEHPETPIIGIDRGERNLIYITVIDSTGKILEQRSLNTIQQEDYQKKLDNREKERV
AARQAWSVVGTIKDLKQGYLSQVIHEIVDLMIHYQAVVVLENLNEGFKSKRTGIAE
KAVYQQFEKMLIDKLNCLVLKDYPAEKVGGVLNPYQLTDQFTSFAKMGTQSGFLFY
VPAPYTSKIDPLTGFVDPFVWKTIKNHESRKHFLEGFDFLHYDVKTGDFILHFKMN
RNLSFQRGLPGFMPAWDIVFEKNETQFDAKGTPFIAGKRIVPVIENHRFTGRYRDL
YPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLK
LQNGISNQDWLAYIQELRN
C334S mutant Cas12a amino acid sequence
SEQ ID NO. 5
MTQFEGFTNLYQVSKTLRFELIPQGKTLKHIQEQGFIEEDKARNDHYKELKPIIDR
IYKTYADQCLQLVQLDWENLSAAIDSYRKEKTEETRNALIEEQATYRNAIHDYFIG
RTDNLTDAINKRHAEIYKGLFKAELENGKVLKQLGTVTTTEHENALLRSEDKETTY
FSGEYENRKNVESAEDISTAIPHRIVQDNFPKEKENCHIFTRLITAVPSLREHFEN
VKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFSKY
KTLLRNENVLETAEALFNELNSIDLTHIFISHKKLETISSALCDHWDTLRNALYER
RISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIK
LEMEPSLSFYNKARNYATKKPYSVEKFKLNFQMPTLASGWDVNKEKNNGAILFVKN
GLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREA
LCKWIDETRDELSKYTKTTSIDLSSLRPSSQYKDLGEYYAELNPLLYHISFQRIAE
KEIMDAVETGKLYLFQIYNKDFAKGHHGKPNLHTLYWTGLFSPENLAKTSIKLNGQ
AELFYRPKSRMKRMAHRLGEKMLNKKLKDQKTPIPDTLYQELYDYVNHRLSHDLSD
EARALLPNVITKEVSHEIIKDRRFTSDKEFFHVPITLNYQAANSPSKFNQRVNAYL
KEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNEGEKSKRTGIAE
KAVYQQFEKMLIDKLNCLVLKDYPAEKVGGVLNPYQLTDQFTSFAKMGTQSGELFY
VPAPYTSKIDPLTGFVDPFVWKTIKNHESRKHFLEGFDFLHYDVKTGDFILHFKMN
RNLSFQRGLPGEMPAWDIVFEKNETQFDAKGTPFIAGKRIVPVIENHRFTGRYRDL
YPANELIALLEEKGIVERDGSNILPKLLENDDSHAIDTMVALIRSVLQMRNSNAAT
GEDYINSPVRDLNGVCFDSRFQNPEWPMDADANGAYHIALKGQLLLNHLKESKDLK
LQNGISNQDWLAYIQELRN
C379S mutant Cas12a amino acid sequence
SEQ ID NO. 6
MTQFEGFTNLYQVSKTLRFELIPQGKTLKHIQEQGFIEEDKARNDHYKELKPIIDR
IYKTYADQCLQLVQLDWENLSAAIDSYRKEKTEETRNALIEEQATYRNAIHDYFIG
RTDNLTDAINKRHAEIYKGLFKAELENGKVLKQLGTVTTTEHENALLRSEDKFTTY
FSGEYENRKNVESAEDISTAIPHRIVQDNFPKEKENCHIFTRLITAVPSLREHFEN
VKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFCKY
KTLLRNENVLETAEALFNELNSIDLTHIFISHKKLETISSALSDHWDTLRNALYER
RISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIK
LEMEPSLSFYNKARNYATKKPYSVEKFKLNFQMPTLASGWDVNKEKNNGAILFVKN
GLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREA
LCKWIDETRDELSKYTKTTSIDLSSLRPSSQYKDLGEYYAELNPLLYHISFQRIAE
KEIMDAVETGKLYLFQIYNKDFAKGHHGKPNLHTLYWTGLFSPENLAKTSIKLNGQ
AELFYRPKSRMKRMAHRLGEKMLNKKLKDQKTPIPDTLYQELYDYVNHRLSHDLSD
EARALLPNVITKEVSHEIIKDRRFTSDKEFFHVPITLNYQAANSPSKFNQRVNAYL
KEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNEGEKSKRTGIAE
KAVYQQFEKMLIDKLNCLVLKDYPAEKVGGVLNPYQLTDQFTSFAKMGTQSGELFY
VPAPYTSKIDPLTGFVDPFVWKTIKNHESRKHFLEGFDFLHYDVKTGDFILHFKMN
RNLSFQRGLPGEMPAWDIVFEKNETQFDAKGTPFIAGKRIVPVIENHRFTGRYRDL
YPANELIALLEEKGIVERDGSNILPKLLENDDSHAIDTMVALIRSVLQMRNSNAAT
GEDYINSPVRDLNGVCFDSRFQNPEWPMDADANGAYHIALKGQLLLNHLKESKDLK
LQNGISNQDWLAYIQELRN
C608S mutant Cas12a amino acid sequence
SEQ ID NO. 7
MTQFEGFTNLYQVSKTLRFELIPQGKTLKHIQEQGFIEEDKARNDHYKELKPIIDR
IYKTYADQCLQLVQLDWENLSAAIDSYRKEKTEETRNALIEEQATYRNAIHDYFIG
RTDNLTDAINKRHAEIYKGLFKAELFNGKVLKQLGTVTTTEHENALLRSFDKFTTY
FSGFYENRKNVFSAEDISTAIPHRIVQDNFPKFKENCHIFTRLITAVPSLREHFEN
VKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFCKY
KTLLRNENVLETAEALFNELNSIDLTHIFISHKKLETISSALCDHWDTLRNALYER
RISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIK
LEMEPSLSFYNKARNYATKKPYSVEKFKLNFQMPTLASGWDVNKEKNNGAILFVKN
GLYYLGIMPKQKGRYKALSFEPTEKTSEGFDKMYYDYFPDAAKMIPKSSTQLKAVT
AHFQTHTTPILLSNNFIEPLEITKEIYDLNNPEKEPKKFQTAYAKKTGDQKGYREA
LCKWIDFTRDFLSKYTKTTSIDLSSLRPSSQYKDLGEYYAELNPLLYHISFQRIAE
KEIMDAVETGKLYLFQIYNKDFAKGHHGKPNLHTLYWTGLFSPENLAKTSIKLNGQ
AELFYRPKSRMKRMAHRLGEKMLNKKLKDQKTPIPDTLYQELYDYVNHRLSHDLSD
EARALLPNVITKEVSHEIIKDRRFTSDKFFFHVPITLNYQAANSPSKFNQRVNAYL
KEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSKRTGIAE
KAVYQQFEKMLIDKLNCLVLKDYPAEKVGGVLNPYQLTDQFTSFAKMGTQSGFLFY
VPAPYTSKIDPLTGFVDPFVWKTIKNHESRKHFLEGFDFLHYDVKTGDFILHFKMN
RNLSFQRGLPGFMPAWDIVFEKNETQFDAKGTPFIAGKRIVPVIENHRFTGRYRDL
YPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLK
LQNGISNQDWLAYIQELRN
C674S mutant Cas12a amino acid sequence
SEQ ID NO. 8
MTQFEGFTNLYQVSKTLRFELIPQGKTLKHIQEQGFIEEDKARNDHYKELKPIIDR
IYKTYADQCLQLVQLDWENLSAAIDSYRKEKTEETRNALIEEQATYRNAIHDYFIG
RTDNLTDAINKRHAEIYKGLFKAELFNGKVLKQLGTVTTTEHENALLRSFDKFTTY
FSGFYENRKNVFSAEDISTAIPHRIVQDNFPKFKENCHIFTRLITAVPSLREHFEN
VKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFCKY
KTLLRNENVLETAEALFNELNSIDLTHIFISHKKLETISSALCDHWDTLRNALYER
RISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIK
LEMEPSLSFYNKARNYATKKPYSVEKFKLNFQMPTLASGWDVNKEKNNGAILFVKN
GLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREA
LSKWIDFTRDFLSKYTKTTSIDLSSLRPSSQYKDLGEYYAELNPLLYHISFQRIAE
KEIMDAVETGKLYLFQIYNKDFAKGHHGKPNLHTLYWTGLFSPENLAKTSIKLNGQ
AELFYRPKSRMKRMAHRLGEKMLNKKLKDQKTPIPDTLYQELYDYVNHRLSHDLSD
EARALLPNVITKEVSHEIIKDRRFTSDKFFFHVPITLNYQAANSPSKFNQRVNAYL
KEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSKRTGIAE
KAVYQQFEKMLIDKLNCLVLKDYPAEKVGGVLNPYQLTDQFTSFAKMGTQSGFLFY
VPAPYTSKIDPLTGFVDPFVWKTIKNHESRKHFLEGFDFLHYDVKTGDFILHFKMN
RNLSFQRGLPGFMPAWDIVFEKNETQFDAKGTPFIAGKRIVPVIENHRFTGRYRDL
YPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLK
LQNGISNQDWLAYIQELRN
C1025S mutant Cas12a amino acid sequence
SEQ ID NO. 9
MTQFEGFTNLYQVSKTLRFELIPQGKTLKHIQEQGFIEEDKARNDHYKELKPIIDR
IYKTYADQCLQLVQLDWENLSAAIDSYRKEKTEETRNALIEEQATYRNAIHDYFIG
RTDNLTDAINKRHAEIYKGLFKAELFNGKVLKQLGTVTTTEHENALLRSFDKFTTY
FSGFYENRKNVFSAEDISTAIPHRIVQDNFPKFKENCHIFTRLITAVPSLREHFEN
VKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFCKY
KTLLRNENVLETAEALFNELNSIDLTHIFISHKKLETISSALCDHWDTLRNALYER
RISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIK
LEMEPSLSFYNKARNYATKKPYSVEKFKLNFQMPTLASGWDVNKEKNNGAILFVKN
GLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREA
LCKWIDFTRDFLSKYTKTTSIDLSSLRPSSQYKDLGEYYAELNPLLYHISFQRIAE
KEIMDAVETGKLYLFQIYNKDFAKGHHGKPNLHTLYWTGLFSPENLAKTSIKLNGQ
AELFYRPKSRMKRMAHRLGEKMLNKKLKDQKTPIPDTLYQELYDYVNHRLSHDLSD
EARALLPNVITKEVSHEIIKDRRFTSDKFFFHVPITLNYQAANSPSKFNQRVNAYL
KEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSKRTGIAE
KAVYQQFEKMLIDKLNSLVLKDYPAEKVGGVLNPYQLTDQFTSFAKMGTQSGFLFY
VPAPYTSKIDPLTGFVDPFVWKTIKNHESRKHFLEGFDFLHYDVKTGDFILHFKMN
RNLSFQRGLPGFMPAWDIVFEKNETQFDAKGTPFIAGKRIVPVIENHRFTGRYRDL
YPANELIALLEEKGIVFRDGSNILPKLLENDDSHAIDTMVALIRSVLQMRNSNAAT
GEDYINSPVRDLNGVCFDSRFQNPEWPMDADANGAYHIALKGQLLLNHLKESKDLK
LQNGISNQDWLAYIQELRN
C1248S mutant Cas12a amino acid sequence
SEQ ID NO. 10
MTQFEGFTNLYQVSKTLRFELIPQGKTLKHIQEQGFIEEDKARNDHYKELKPIIDR
IYKTYADQCLQLVQLDWENLSAAIDSYRKEKTEETRNALIEEQATYRNAIHDYFIG
RTDNLTDAINKRHAEIYKGLFKAELFNGKVLKQLGTVTTTEHENALLRSFDKFTTY
FSGFYENRKNVFSAEDISTAIPHRIVQDNFPKFKENCHIFTRLITAVPSLREHFEN
VKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFCKY
KTLLRNENVLETAEALFNELNSIDLTHIFISHKKLETISSALCDHWDTLRNALYER
RISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIK
LEMEPSLSFYNKARNYATKKPYSVEKFKLNFQMPTLASGWDVNKEKNNGAILFVKN
GLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREA
LCKWIDFTRDFLSKYTKTTSIDLSSLRPSSQYKDLGEYYAELNPLLYHISFQRIAE
KEIMDAVETGKLYLFQIYNKDFAKGHHGKPNLHTLYWTGLFSPENLAKTSIKLNGQ
AELFYRPKSRMKRMAHRLGEKMLNKKLKDQKTPIPDTLYQELYDYVNHRLSHDLSD
EARALLPNVITKEVSHEIIKDRRFTSDKFFFHVPITLNYQAANSPSKFNQRVNAYL
KEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSKRTGIAE
KAVYQQFEKMLIDKLNCLVLKDYPAEKVGGVLNPYQLTDQFTSFAKMGTQSGFLFY
VPAPYTSKIDPLTGFVDPFVWKTIKNHESRKHFLEGFDFLHYDVKTGDFILHFKMN
RNLSFQRGLPGFMPAWDIVFEKNETQFDAKGTPFIAGKRIVPVIENHRFTGRYRDL
YPANELIALLEEKGIVFRDGSNILPKLLENDDSHAIDTMVALIRSVLQMRNSNAAT
GEDYINSPVRDLNGVSFDSRFQNPEWPMDADANGAYHIALKGQLLLNHLKESKDLK
LQNGISNQDWLAYIQELRN
Multi-combination mutant C205S, C379S, C674S, C1248S mutant
Cas12a amino acid sequence
SEQ ID NO. 11
MTQFEGFTNLYQVSKTLRFELIPQGKTLKHIQEQGFIEEDKARNDHYKELKPIIDR
IYKTYADQCLQLVQLDWENLSAAIDSYRKEKTEETRNALIEEQATYRNAIHDYFIG
RTDNLTDAINKRHAEIYKGLFKAELFNGKVLKQLGTVTTTEHENALLRSFDKFTTY
FSGFYENRKNVFSAEDISTAIPHRIVQDNFPKFKENSHIFTRLITAVPSLREHFEN
VKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFCKY
KTLLRNENVLETAEALFNELNSIDLTHIFISHKKLETISSALSDHWDTLRNALYER
RISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIK
LEMEPSLSFYNKARNYATKKPYSVEKFKLNFQMPTLASGWDVNKEKNNGAILFVKN
GLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREA
LSKWIDFTRDFLSKYTKTTSIDLSSLRPSSQYKDLGEYYAELNPLLYHISFQRIAE
KEIMDAVETGKLYLFQIYNKDFAKGHHGKPNLHTLYWTGLFSPENLAKTSIKLNGQ
AELFYRPKSRMKRMAHRLGEKMLNKKLKDQKTPIPDTLYQELYDYVNHRLSHDLSD
EARALLPNVITKEVSHEIIKDRRFTSDKFFFHVPITLNYQAANSPSKFNQRVNAYL
KEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSKRTGIAE
KAVYQQFEKMLIDKLNCLVLKDYPAEKVGGVLNPYQLTDQFTSFAKMGTQSGFLFY
VPAPYTSKIDPLTGFVDPFVWKTIKNHESRKHFLEGFDFLHYDVKTGDFILHFKMN
RNLSFQRGLPGFMPAWDIVFEKNETQFDAKGTPFIAGKRIVPVIENHRFTGRYRDL
YPANELIALLEEKGIVFRDGSNILPKLLENDDSHAIDTMVALIRSVLQMRNSNAAT
GEDYINSPVRDLNGVSFDSRFQNPEWPMDADANGAYHIALKGQLLLNHLKESKDLK
LQNGISNQDWLAYIQELRN
Multi-combination mutant C65S, C205S, C334S, C379S, C674S,
C1248S mutant Cas12a amino acid sequence
SEQ ID NO. 12.
MTQFEGFTNLYQVSKTLRFELIPQGKTLKHIQEQGFIEEDKARNDHYKELKPIIDR
IYKTYADQSLQLVQLDWENLSAAIDSYRKEKTEETRNALIEEQATYRNAIHDYFIG
RTDNLTDAINKRHAEIYKGLFKAELFNGKVLKQLGTVTTTEHENALLRSFDKFTTY
FSGFYENRKNVFSAEDISTAIPHRIVQDNFPKFKENSHIFTRLITAVPSLREHFEN
VKKAIGIFVSTSIEEVFSFPFYNQLLTQTQIDLYNQLLGGISREAGTEKIKGLNEV
LNLAIQKNDETAHIIASLPHRFIPLFKQILSDRNTLSFILEEFKSDEEVIQSFSKY
KTLLRNENVLETAEALFNELNSIDLTHIFISHKKLETISSALSDHWDTLRNALYER
RISELTGKITKSAKEKVQRSLKHEDINLQEIISAAGKELSEAFKQKTSEILSHAHA
ALDQPLPTTLKKQEEKEILKSQLDSLLGLYHLLDWFAVDESNEVDPEFSARLTGIK
LEMEPSLSFYNKARNYATKKPYSVEKFKLNFQMPTLASGWDVNKEKNNGAILFVKN
GLYYLGIMPKQKGRYKALSFEPTEKTSEGFDKMYYDYFPDAAKMIPKCSTQLKAVT
AHFQTHTTPILLSNNFIEPLEITKEIYDLNNPEKEPKKFQTAYAKKTGDQKGYREA
LSKWIDFTRDFLSKYTKTTSIDLSSLRPSSQYKDLGEYYAELNPLLYHISFQRIAE
KEIMDAVETGKLYLFQIYNKDFAKGHHGKPNLHTLYWTGLFSPENLAKTSIKLNGQ
AELFYRPKSRMKRMAHRLGEKMLNKKLKDQKTPIPDTLYQELYDYVNHRLSHDLSD
EARALLPNVITKEVSHEIIKDRRFTSDKFFFHVPITLNYQAANSPSKFNQRVNAYL
KEHPETPIIGIDRGERNLIYITVIDSTGKILEQRSLNTIQQFDYQKKLDNREKERV
AARQAWSVVGTIKDLKQGYLSQVIHEIVDLMIHYQAVVVLENLNFGFKSKRTGIAE
KAVYQQFEKMLIDKLNCLVLKDYPAEKVGGVLNPYQLTDQFTSFAKMGTQSGFLFY
VPAPYTSKIDPLTGFVDPFVWKTIKNHESRKHFLEGFDFLHYDVKTGDFILHFKMN
RNLSFQRGLPGFMPAWDIVFEKNETQFDAKGTPFIAGKRIVPVIENHRFTGRYRDL
YPANELIALLEEKGIVFRDGSNILPKLLENDDSHAIDTMVALIRSVLQMRNSNAAT
GEDYINSPVRDLNGVSFDSRFQNPEWPMDADANGAYHIALKGQLLLNHLKESKDLK
LQNGISNQDWLAYIQELRN
C65S Cas12a mutant DNA sequence
SEQ ID NO. 13
ATGACCCAGTTCGAGGGCTTCACCAACCTGTACCAGGTGTCCAAGACCCTGA
GATTCGAGCTGATCCCCCAGGGCAAGACACTGAAGCACATCCAGGAACAGG
GCTTCATCGAAGAGGACAAGGCCCGGAACGACCACTACAAAGAGCTGAAGC
CCATCATCGACCGGATCTACAAGACCTACGCCGACCAGTCTCTGCAGCTGGT
GCAGCTGGACTGGGAGAATCTGAGCGCCGCCATCGACAGCTACCGGAAAGA
GAAAACCGAGGAAACCCGGAACGCCCTGATCGAGGAACAGGCCACCTACAG
AAACGCCATCCACGACTACTTCATCGGCCGGACCGACAACCTGACCGACGCC
ATCAACAAGCGGCACGCCGAGATCTATAAGGGCCTGTTCAAGGCCGAGCTGT
TCAACGGCAAGGTGCTGAAGCAGCTGGGCACCGTGACCACCACCGAGCACG
AAAACGCCCTGCTGCGGAGCTTCGACAAGTTCACCACCTACTTCAGCGGCTT
CTACGAGAACCGGAAGAACGTGTTCAGCGCCGAGGACATCAGCACCGCCATC
CCCCACAGAATCGTGCAGGACAACTTCCCCAAGTTCAAAGAGAACTGCCACA
TCTTCACCCGGCTGATCACCGCCGTGCCCAGCCTGAGAGAACACTTCGAGAA
CGTGAAGAAGGCCATCGGCATCTTCGTGTCCACCAGCATCGAGGAAGTGTTC
AGCTTCCCATTCTACAACCAGCTGCTGACCCAGACCCAGATCGACCTGTATA
ATCAGCTGCTGGGCGGCATCAGCAGAGAGGCCGGCACCGAGAAGATCAAGG
GCCTGAACGAAGTGCTGAACCTGGCCATCCAGAAGAACGACGAGACAGCCC
ACATCATTGCCAGCCTGCCCCACCGGTTCATCCCTCTGTTCAAGCAGATCCTG
AGCGACAGAAACACCCTGAGCTTCATCCTGGAAGAGTTCAAGTCCGATGAGG
AAGTGATCCAGAGCTTCTGCAAGTATAAGACCCTGCTGAGGAACGAGAATGT
GCTGGAAACCGCCGAGGCCCTGTTCAATGAGCTGAACAGCATCGACCTGACC
CACATCTTTATCAGCCACAAGAAGCTGGAAACAATCAGCAGCGCCCTGTGCG
ACCACTGGGACACACTGCGGAATGCCCTGTACGAGCGGCGGATCTCTGAGCT
GACCGGCAAGATCACCAAGAGCGCCAAAGAAAAGGTGCAGCGGAGCCTGAA
GCACGAGGATATCAACCTGCAGGAAATCATCAGCGCCGCTGGCAAAGAACT
GAGCGAGGCCTTTAAGCAGAAAACCAGCGAGATCCTGTCCCACGCCCACGCC
GCACTGGATCAGCCTCTGCCTACCACCCTGAAGAAGCAGGAAGAGAAAGAG
ATCCTGAAGTCCCAGCTGGACAGCCTGCTGGGCCTGTACCATCTGCTGGATTG
GTTCGCCGTGGACGAGAGCAACGAGGTGGACCCCGAGTTCTCCGCCAGACTG
ACAGGCATCAAACTGGAAATGGAACCCAGCCTGTCCTTCTACAACAAGGCCA
GAAACTACGCCACCAAGAAACCCTACAGCGTGGAAAAGTTTAAGCTGAACTT
CCAGATGCCCACCCTGGCCAGCGGCTGGGACGTGAACAAAGAGAAGAACAA
CGGCGCCATCCTGTTCGTGAAGAACGGACTGTACTACCTGGGCATCATGCCT
AAGCAGAAGGGCAGATACAAGGCCCTGTCCTTTGAGCCCACCGAAAAGACC
AGCGAGGGCTTTGACAAGATGTACTACGATTACTTCCCCGACGCCGCCAAGA
TGATCCCCAAGTGCAGCACCCAGCTGAAGGCCGTGACCGCCCACTTTCAGAC
CCACACCACCCCCATCCTGCTGAGCAACAACTTCATCGAGCCCCTGGAAATC
ACCAAAGAGATCTACGACCTGAACAACCCCGAGAAAGAGCCCAAGAAGTTC
CAGACCGCCTACGCCAAGAAAACCGGCGACCAGAAGGGCTACCGCGAGGCT
CTGTGCAAGTGGATCGACTTTACCCGGGACTTCCTGAGCAAGTACACCAAGA
CCACCTCCATCGATCTGAGCAGCCTGCGGCCCAGCTCCCAGTACAAGGATCT
GGGCGAGTACTACGCCGAGCTGAACCCTCTGCTGTACCACATCAGCTTCCAG
CGGATCGCCGAAAAAGAAATCATGGACGCCGTGGAAACCGGCAAGCTGTAC
CTGTTCCAGATCTATAACAAGGACTTCGCCAAGGGCCACCACGGCAAGCCCA
ATCTGCACACCCTGTACTGGACCGGCCTGTTTAGCCCCGAGAATCTGGCCAA
GACCAGCATCAAGCTGAACGGCCAGGCCGAACTGTTTTACCGGCCCAAGAGC
CGGATGAAGCGGATGGCCCATAGACTGGGCGAGAAGATGCTGAACAAGAAA
CTGAAGGACCAGAAAACCCCTATCCCCGACACACTGTATCAGGAACTGTACG
ACTACGTGAACCACCGGCTGAGCCACGACCTGTCCGACGAAGCTAGAGCACT
GCTGCCCAACGTGATCACAAAAGAGGTGTCCCACGAGATCATCAAGGACCGG
CGGTTTACCTCCGATAAGTTCTTCTTCCACGTGCCCATCACCCTGAACTACCA
GGCCGCCAACAGCCCCAGCAAGTTCAACCAGAGAGTGAACGCCTACCTGAA
AGAGCACCCCGAGACACCCATCATTGGCATCGACAGAGGCGAGCGGAACCT
GATCTACATCACCGTGATCGACAGCACAGGCAAAATCCTGGAACAGAGAAG
CCTGAACACCATCCAGCAGTTCGACTACCAGAAGAAACTGGACAACCGGGA
AAAAGAACGGGTGGCCGCCAGACAGGCTTGGAGCGTCGTGGGCACCATTAA
GGACCTGAAGCAGGGCTACCTGAGCCAAGTGATTCACGAGATCGTGGACCTG
ATGATCCACTATCAGGCTGTGGTGGTGCTGGAAAACCTGAACTTCGGCTTCA
AGAGCAAGCGGACCGGAATCGCCGAGAAAGCCGTGTACCAGCAGTTTGAGA
AAATGCTGATCGACAAGCTGAATTGCCTGGTGCTGAAAGACTACCCCGCTGA
GAAAGTGGGAGGCGTGCTGAATCCCTACCAGCTGACCGACCAGTTCACCTCC
TTTGCCAAGATGGGAACCCAGAGCGGCTTCCTGTTCTACGTGCCAGCCCCCTA
CACCAGCAAGATCGACCCTCTGACCGGCTTCGTGGACCCCTTCGTGTGGAAA
ACCATCAAGAACCACGAGTCCCGGAAGCACTTCCTGGAAGGCTTTGACTTCC
TGCACTACGACGTGAAAACAGGCGATTTCATCCTGCACTTCAAGATGAATCG
GAATCTGTCCTTCCAGAGGGGCCTGCCCGGCTTCATGCCTGCCTGGGATATCG
TGTTCGAGAAGAATGAGACACAGTTCGACGCCAAGGGAACCCCCTTTATCGC
CGGCAAGAGGATCGTGCCTGTGATCGAGAACCACAGATTCACCGGCAGATAC
CGGGACCTGTACCCCGCCAACGAGCTGATTGCCCTGCTGGAAGAGAAGGGCA
TCGTGTTCCGGGACGGCAGCAACATCCTGCCCAAGCTGCTGGAAAATGACGA
CAGCCACGCCATCGATACCATGGTGGCACTGATCCGCAGCGTGCTGCAGATG
CGGAACAGCAATGCCGCCACCGGCGAGGACTACATCAATAGCCCAGTGCGG
GACCTGAACGGCGTGTGCTTCGACAGCAGATTCCAGAACCCCGAGTGGCCCA
TGGATGCCGACGCCAATGGCGCCTACCACATTGCCCTGAAGGGACAGCTGCT
GCTGAACCATCTGAAAGAGAGCAAAGACCTGAAACTGCAGAACGGCATCTC
CAACCAGGACTGGCTGGCCTATATCCAGGAACTGCGGAAC
C205S Cas12a mutant DNA sequence
SEQ ID NO. 14
ATGACCCAGTTCGAGGGCTTCACCAACCTGTACCAGGTGTCCAAGACCCTGA
GATTCGAGCTGATCCCCCAGGGCAAGACACTGAAGCACATCCAGGAACAGG
GCTTCATCGAAGAGGACAAGGCCCGGAACGACCACTACAAAGAGCTGAAGC
CCATCATCGACCGGATCTACAAGACCTACGCCGACCAGTGCCTGCAGCTGGT
GCAGCTGGACTGGGAGAATCTGAGCGCCGCCATCGACAGCTACCGGAAAGA
GAAAACCGAGGAAACCCGGAACGCCCTGATCGAGGAACAGGCCACCTACAG
AAACGCCATCCACGACTACTTCATCGGCCGGACCGACAACCTGACCGACGCC
ATCAACAAGCGGCACGCCGAGATCTATAAGGGCCTGTTCAAGGCCGAGCTGT
TCAACGGCAAGGTGCTGAAGCAGCTGGGCACCGTGACCACCACCGAGCACG
AAAACGCCCTGCTGCGGAGCTTCGACAAGTTCACCACCTACTTCAGCGGCTT
CTACGAGAACCGGAAGAACGTGTTCAGCGCCGAGGACATCAGCACCGCCATC
CCCCACAGAATCGTGCAGGACAACTTCCCCAAGTTCAAAGAGAACTCTCACA
TCTTCACCCGGCTGATCACCGCCGTGCCCAGCCTGAGAGAACACTTCGAGAA
CGTGAAGAAGGCCATCGGCATCTTCGTGTCCACCAGCATCGAGGAAGTGTTC
AGCTTCCCATTCTACAACCAGCTGCTGACCCAGACCCAGATCGACCTGTATA
ATCAGCTGCTGGGCGGCATCAGCAGAGAGGCCGGCACCGAGAAGATCAAGG
GCCTGAACGAAGTGCTGAACCTGGCCATCCAGAAGAACGACGAGACAGCCC
ACATCATTGCCAGCCTGCCCCACCGGTTCATCCCTCTGTTCAAGCAGATCCTG
AGCGACAGAAACACCCTGAGCTTCATCCTGGAAGAGTTCAAGTCCGATGAGG
AAGTGATCCAGAGCTTCTGCAAGTATAAGACCCTGCTGAGGAACGAGAATGT
GCTGGAAACCGCCGAGGCCCTGTTCAATGAGCTGAACAGCATCGACCTGACC
CACATCTTTATCAGCCACAAGAAGCTGGAAACAATCAGCAGCGCCCTGTGCG
ACCACTGGGACACACTGCGGAATGCCCTGTACGAGCGGCGGATCTCTGAGCT
GACCGGCAAGATCACCAAGAGCGCCAAAGAAAAGGTGCAGCGGAGCCTGAA
GCACGAGGATATCAACCTGCAGGAAATCATCAGCGCCGCTGGCAAAGAACT
GAGCGAGGCCTTTAAGCAGAAAACCAGCGAGATCCTGTCCCACGCCCACGCC
GCACTGGATCAGCCTCTGCCTACCACCCTGAAGAAGCAGGAAGAGAAAGAG
ATCCTGAAGTCCCAGCTGGACAGCCTGCTGGGCCTGTACCATCTGCTGGATTG
GTTCGCCGTGGACGAGAGCAACGAGGTGGACCCCGAGTTCTCCGCCAGACTG
ACAGGCATCAAACTGGAAATGGAACCCAGCCTGTCCTTCTACAACAAGGCCA
GAAACTACGCCACCAAGAAACCCTACAGCGTGGAAAAGTTTAAGCTGAACTT
CCAGATGCCCACCCTGGCCAGCGGCTGGGACGTGAACAAAGAGAAGAACAA
CGGCGCCATCCTGTTCGTGAAGAACGGACTGTACTACCTGGGCATCATGCCT
AAGCAGAAGGGCAGATACAAGGCCCTGTCCTTTGAGCCCACCGAAAAGACC
AGCGAGGGCTTTGACAAGATGTACTACGATTACTTCCCCGACGCCGCCAAGA
TGATCCCCAAGTGCAGCACCCAGCTGAAGGCCGTGACCGCCCACTTTCAGAC
CCACACCACCCCCATCCTGCTGAGCAACAACTTCATCGAGCCCCTGGAAATC
ACCAAAGAGATCTACGACCTGAACAACCCCGAGAAAGAGCCCAAGAAGTTC
CAGACCGCCTACGCCAAGAAAACCGGCGACCAGAAGGGCTACCGCGAGGCT
CTGTGCAAGTGGATCGACTTTACCCGGGACTTCCTGAGCAAGTACACCAAGA
CCACCTCCATCGATCTGAGCAGCCTGCGGCCCAGCTCCCAGTACAAGGATCT
GGGCGAGTACTACGCCGAGCTGAACCCTCTGCTGTACCACATCAGCTTCCAG
CGGATCGCCGAAAAAGAAATCATGGACGCCGTGGAAACCGGCAAGCTGTAC
CTGTTCCAGATCTATAACAAGGACTTCGCCAAGGGCCACCACGGCAAGCCCA
ATCTGCACACCCTGTACTGGACCGGCCTGTTTAGCCCCGAGAATCTGGCCAA
GACCAGCATCAAGCTGAACGGCCAGGCCGAACTGTTTTACCGGCCCAAGAGC
CGGATGAAGCGGATGGCCCATAGACTGGGCGAGAAGATGCTGAACAAGAAA
CTGAAGGACCAGAAAACCCCTATCCCCGACACACTGTATCAGGAACTGTACG
ACTACGTGAACCACCGGCTGAGCCACGACCTGTCCGACGAAGCTAGAGCACT
GCTGCCCAACGTGATCACAAAAGAGGTGTCCCACGAGATCATCAAGGACCGG
CGGTTTACCTCCGATAAGTTCTTCTTCCACGTGCCCATCACCCTGAACTACCA
GGCCGCCAACAGCCCCAGCAAGTTCAACCAGAGAGTGAACGCCTACCTGAA
AGAGCACCCCGAGACACCCATCATTGGCATCGACAGAGGCGAGCGGAACCT
GATCTACATCACCGTGATCGACAGCACAGGCAAAATCCTGGAACAGAGAAG
CCTGAACACCATCCAGCAGTTCGACTACCAGAAGAAACTGGACAACCGGGA
AAAAGAACGGGTGGCCGCCAGACAGGCTTGGAGCGTCGTGGGCACCATTAA
GGACCTGAAGCAGGGCTACCTGAGCCAAGTGATTCACGAGATCGTGGACCTG
ATGATCCACTATCAGGCTGTGGTGGTGCTGGAAAACCTGAACTTCGGCTTCA
AGAGCAAGCGGACCGGAATCGCCGAGAAAGCCGTGTACCAGCAGTTTGAGA
AAATGCTGATCGACAAGCTGAATTGCCTGGTGCTGAAAGACTACCCCGCTGA
GAAAGTGGGAGGCGTGCTGAATCCCTACCAGCTGACCGACCAGTTCACCTCC
TTTGCCAAGATGGGAACCCAGAGCGGCTTCCTGTTCTACGTGCCAGCCCCCTA
CACCAGCAAGATCGACCCTCTGACCGGCTTCGTGGACCCCTTCGTGTGGAAA
ACCATCAAGAACCACGAGTCCCGGAAGCACTTCCTGGAAGGCTTTGACTTCC
TGCACTACGACGTGAAAACAGGCGATTTCATCCTGCACTTCAAGATGAATCG
GAATCTGTCCTTCCAGAGGGGCCTGCCCGGCTTCATGCCTGCCTGGGATATCG
TGTTCGAGAAGAATGAGACACAGTTCGACGCCAAGGGAACCCCCTTTATCGC
CGGCAAGAGGATCGTGCCTGTGATCGAGAACCACAGATTCACCGGCAGATAC
CGGGACCTGTACCCCGCCAACGAGCTGATTGCCCTGCTGGAAGAGAAGGGCA
TCGTGTTCCGGGACGGCAGCAACATCCTGCCCAAGCTGCTGGAAAATGACGA
CAGCCACGCCATCGATACCATGGTGGCACTGATCCGCAGCGTGCTGCAGATG
CGGAACAGCAATGCCGCCACCGGCGAGGACTACATCAATAGCCCAGTGCGG
GACCTGAACGGCGTGTGCTTCGACAGCAGATTCCAGAACCCCGAGTGGCCCA
TGGATGCCGACGCCAATGGCGCCTACCACATTGCCCTGAAGGGACAGCTGCT
GCTGAACCATCTGAAAGAGAGCAAAGACCTGAAACTGCAGAACGGCATCTC
CAACCAGGACTGGCTGGCCTATATCCAGGAACTGCGGAAC
C334S Cas12a mutant DNA sequence
SEQ ID NO. 15
ATGACCCAGTTCGAGGGCTTCACCAACCTGTACCAGGTGTCCAAGACCCTGA
GATTCGAGCTGATCCCCCAGGGCAAGACACTGAAGCACATCCAGGAACAGG
GCTTCATCGAAGAGGACAAGGCCCGGAACGACCACTACAAAGAGCTGAAGC
CCATCATCGACCGGATCTACAAGACCTACGCCGACCAGTGCCTGCAGCTGGT
GCAGCTGGACTGGGAGAATCTGAGCGCCGCCATCGACAGCTACCGGAAAGA
GAAAACCGAGGAAACCCGGAACGCCCTGATCGAGGAACAGGCCACCTACAG
AAACGCCATCCACGACTACTTCATCGGCCGGACCGACAACCTGACCGACGCC
ATCAACAAGCGGCACGCCGAGATCTATAAGGGCCTGTTCAAGGCCGAGCTGT
TCAACGGCAAGGTGCTGAAGCAGCTGGGCACCGTGACCACCACCGAGCACG
AAAACGCCCTGCTGCGGAGCTTCGACAAGTTCACCACCTACTTCAGCGGCTT
CTACGAGAACCGGAAGAACGTGTTCAGCGCCGAGGACATCAGCACCGCCATC
CCCCACAGAATCGTGCAGGACAACTTCCCCAAGTTCAAAGAGAACTGCCACA
TCTTCACCCGGCTGATCACCGCCGTGCCCAGCCTGAGAGAACACTTCGAGAA
CGTGAAGAAGGCCATCGGCATCTTCGTGTCCACCAGCATCGAGGAAGTGTTC
AGCTTCCCATTCTACAACCAGCTGCTGACCCAGACCCAGATCGACCTGTATA
ATCAGCTGCTGGGCGGCATCAGCAGAGAGGCCGGCACCGAGAAGATCAAGG
GCCTGAACGAAGTGCTGAACCTGGCCATCCAGAAGAACGACGAGACAGCCC
ACATCATTGCCAGCCTGCCCCACCGGTTCATCCCTCTGTTCAAGCAGATCCTG
AGCGACAGAAACACCCTGAGCTTCATCCTGGAAGAGTTCAAGTCCGATGAGG
AAGTGATCCAGAGCTTCTCTAAGTATAAGACCCTGCTGAGGAACGAGAATGT
GCTGGAAACCGCCGAGGCCCTGTTCAATGAGCTGAACAGCATCGACCTGACC
CACATCTTTATCAGCCACAAGAAGCTGGAAACAATCAGCAGCGCCCTGTGCG
ACCACTGGGACACACTGCGGAATGCCCTGTACGAGCGGCGGATCTCTGAGCT
GACCGGCAAGATCACCAAGAGCGCCAAAGAAAAGGTGCAGCGGAGCCTGAA
GCACGAGGATATCAACCTGCAGGAAATCATCAGCGCCGCTGGCAAAGAACT
GAGCGAGGCCTTTAAGCAGAAAACCAGCGAGATCCTGTCCCACGCCCACGCC
GCACTGGATCAGCCTCTGCCTACCACCCTGAAGAAGCAGGAAGAGAAAGAG
ATCCTGAAGTCCCAGCTGGACAGCCTGCTGGGCCTGTACCATCTGCTGGATTG
GTTCGCCGTGGACGAGAGCAACGAGGTGGACCCCGAGTTCTCCGCCAGACTG
ACAGGCATCAAACTGGAAATGGAACCCAGCCTGTCCTTCTACAACAAGGCCA
GAAACTACGCCACCAAGAAACCCTACAGCGTGGAAAAGTTTAAGCTGAACTT
CCAGATGCCCACCCTGGCCAGCGGCTGGGACGTGAACAAAGAGAAGAACAA
CGGCGCCATCCTGTTCGTGAAGAACGGACTGTACTACCTGGGCATCATGCCT
AAGCAGAAGGGCAGATACAAGGCCCTGTCCTTTGAGCCCACCGAAAAGACC
AGCGAGGGCTTTGACAAGATGTACTACGATTACTTCCCCGACGCCGCCAAGA
TGATCCCCAAGTGCAGCACCCAGCTGAAGGCCGTGACCGCCCACTTTCAGAC
CCACACCACCCCCATCCTGCTGAGCAACAACTTCATCGAGCCCCTGGAAATC
ACCAAAGAGATCTACGACCTGAACAACCCCGAGAAAGAGCCCAAGAAGTTC
CAGACCGCCTACGCCAAGAAAACCGGCGACCAGAAGGGCTACCGCGAGGCT
CTGTGCAAGTGGATCGACTTTACCCGGGACTTCCTGAGCAAGTACACCAAGA
CCACCTCCATCGATCTGAGCAGCCTGCGGCCCAGCTCCCAGTACAAGGATCT
GGGCGAGTACTACGCCGAGCTGAACCCTCTGCTGTACCACATCAGCTTCCAG
CGGATCGCCGAAAAAGAAATCATGGACGCCGTGGAAACCGGCAAGCTGTAC
CTGTTCCAGATCTATAACAAGGACTTCGCCAAGGGCCACCACGGCAAGCCCA
ATCTGCACACCCTGTACTGGACCGGCCTGTTTAGCCCCGAGAATCTGGCCAA
GACCAGCATCAAGCTGAACGGCCAGGCCGAACTGTTTTACCGGCCCAAGAGC
CGGATGAAGCGGATGGCCCATAGACTGGGCGAGAAGATGCTGAACAAGAAA
CTGAAGGACCAGAAAACCCCTATCCCCGACACACTGTATCAGGAACTGTACG
ACTACGTGAACCACCGGCTGAGCCACGACCTGTCCGACGAAGCTAGAGCACT
GCTGCCCAACGTGATCACAAAAGAGGTGTCCCACGAGATCATCAAGGACCGG
CGGTTTACCTCCGATAAGTTCTTCTTCCACGTGCCCATCACCCTGAACTACCA
GGCCGCCAACAGCCCCAGCAAGTTCAACCAGAGAGTGAACGCCTACCTGAA
AGAGCACCCCGAGACACCCATCATTGGCATCGACAGAGGCGAGCGGAACCT
GATCTACATCACCGTGATCGACAGCACAGGCAAAATCCTGGAACAGAGAAG
CCTGAACACCATCCAGCAGTTCGACTACCAGAAGAAACTGGACAACCGGGA
AAAAGAACGGGTGGCCGCCAGACAGGCTTGGAGCGTCGTGGGCACCATTAA
GGACCTGAAGCAGGGCTACCTGAGCCAAGTGATTCACGAGATCGTGGACCTG
ATGATCCACTATCAGGCTGTGGTGGTGCTGGAAAACCTGAACTTCGGCTTCA
AGAGCAAGCGGACCGGAATCGCCGAGAAAGCCGTGTACCAGCAGTTTGAGA
AAATGCTGATCGACAAGCTGAATTGCCTGGTGCTGAAAGACTACCCCGCTGA
GAAAGTGGGAGGCGTGCTGAATCCCTACCAGCTGACCGACCAGTTCACCTCC
TTTGCCAAGATGGGAACCCAGAGCGGCTTCCTGTTCTACGTGCCAGCCCCCTA
CACCAGCAAGATCGACCCTCTGACCGGCTTCGTGGACCCCTTCGTGTGGAAA
ACCATCAAGAACCACGAGTCCCGGAAGCACTTCCTGGAAGGCTTTGACTTCC
TGCACTACGACGTGAAAACAGGCGATTTCATCCTGCACTTCAAGATGAATCG
GAATCTGTCCTTCCAGAGGGGCCTGCCCGGCTTCATGCCTGCCTGGGATATCG
TGTTCGAGAAGAATGAGACACAGTTCGACGCCAAGGGAACCCCCTTTATCGC
CGGCAAGAGGATCGTGCCTGTGATCGAGAACCACAGATTCACCGGCAGATAC
CGGGACCTGTACCCCGCCAACGAGCTGATTGCCCTGCTGGAAGAGAAGGGCA
TCGTGTTCCGGGACGGCAGCAACATCCTGCCCAAGCTGCTGGAAAATGACGA
CAGCCACGCCATCGATACCATGGTGGCACTGATCCGCAGCGTGCTGCAGATG
CGGAACAGCAATGCCGCCACCGGCGAGGACTACATCAATAGCCCAGTGCGG
GACCTGAACGGCGTGTGCTTCGACAGCAGATTCCAGAACCCCGAGTGGCCCA
TGGATGCCGACGCCAATGGCGCCTACCACATTGCCCTGAAGGGACAGCTGCT
GCTGAACCATCTGAAAGAGAGCAAAGACCTGAAACTGCAGAACGGCATCTC
CAACCAGGACTGGCTGGCCTATATCCAGGAACTGCGGAAC
C379S mutant Cas12a DNA sequence
SEQ ID NO. 16
ATGACCCAGTTCGAGGGCTTCACCAACCTGTACCAGGTGTCCAAGACCCTGA
GATTCGAGCTGATCCCCCAGGGCAAGACACTGAAGCACATCCAGGAACAGG
GCTTCATCGAAGAGGACAAGGCCCGGAACGACCACTACAAAGAGCTGAAGC
CCATCATCGACCGGATCTACAAGACCTACGCCGACCAGTGCCTGCAGCTGGT
GCAGCTGGACTGGGAGAATCTGAGCGCCGCCATCGACAGCTACCGGAAAGA
GAAAACCGAGGAAACCCGGAACGCCCTGATCGAGGAACAGGCCACCTACAG
AAACGCCATCCACGACTACTTCATCGGCCGGACCGACAACCTGACCGACGCC
ATCAACAAGCGGCACGCCGAGATCTATAAGGGCCTGTTCAAGGCCGAGCTGT
TCAACGGCAAGGTGCTGAAGCAGCTGGGCACCGTGACCACCACCGAGCACG
AAAACGCCCTGCTGCGGAGCTTCGACAAGTTCACCACCTACTTCAGCGGCTT
CTACGAGAACCGGAAGAACGTGTTCAGCGCCGAGGACATCAGCACCGCCATC
CCCCACAGAATCGTGCAGGACAACTTCCCCAAGTTCAAAGAGAACTGCCACA
TCTTCACCCGGCTGATCACCGCCGTGCCCAGCCTGAGAGAACACTTCGAGAA
CGTGAAGAAGGCCATCGGCATCTTCGTGTCCACCAGCATCGAGGAAGTGTTC
AGCTTCCCATTCTACAACCAGCTGCTGACCCAGACCCAGATCGACCTGTATA
ATCAGCTGCTGGGCGGCATCAGCAGAGAGGCCGGCACCGAGAAGATCAAGG
GCCTGAACGAAGTGCTGAACCTGGCCATCCAGAAGAACGACGAGACAGCCC
ACATCATTGCCAGCCTGCCCCACCGGTTCATCCCTCTGTTCAAGCAGATCCTG
AGCGACAGAAACACCCTGAGCTTCATCCTGGAAGAGTTCAAGTCCGATGAGG
AAGTGATCCAGAGCTTCTGCAAGTATAAGACCCTGCTGAGGAACGAGAATGT
GCTGGAAACCGCCGAGGCCCTGTTCAATGAGCTGAACAGCATCGACCTGACC
CACATCTTTATCAGCCACAAGAAGCTGGAAACAATCAGCAGCGCCCTGTCTG
ACCACTGGGACACACTGCGGAATGCCCTGTACGAGCGGCGGATCTCTGAGCT
GACCGGCAAGATCACCAAGAGCGCCAAAGAAAAGGTGCAGCGGAGCCTGAA
GCACGAGGATATCAACCTGCAGGAAATCATCAGCGCCGCTGGCAAAGAACT
GAGCGAGGCCTTTAAGCAGAAAACCAGCGAGATCCTGTCCCACGCCCACGCC
GCACTGGATCAGCCTCTGCCTACCACCCTGAAGAAGCAGGAAGAGAAAGAG
ATCCTGAAGTCCCAGCTGGACAGCCTGCTGGGCCTGTACCATCTGCTGGATTG
GTTCGCCGTGGACGAGAGCAACGAGGTGGACCCCGAGTTCTCCGCCAGACTG
ACAGGCATCAAACTGGAAATGGAACCCAGCCTGTCCTTCTACAACAAGGCCA
GAAACTACGCCACCAAGAAACCCTACAGCGTGGAAAAGTTTAAGCTGAACTT
CCAGATGCCCACCCTGGCCAGCGGCTGGGACGTGAACAAAGAGAAGAACAA
CGGCGCCATCCTGTTCGTGAAGAACGGACTGTACTACCTGGGCATCATGCCT
AAGCAGAAGGGCAGATACAAGGCCCTGTCCTTTGAGCCCACCGAAAAGACC
AGCGAGGGCTTTGACAAGATGTACTACGATTACTTCCCCGACGCCGCCAAGA
TGATCCCCAAGTGCAGCACCCAGCTGAAGGCCGTGACCGCCCACTTTCAGAC
CCACACCACCCCCATCCTGCTGAGCAACAACTTCATCGAGCCCCTGGAAATC
ACCAAAGAGATCTACGACCTGAACAACCCCGAGAAAGAGCCCAAGAAGTTC
CAGACCGCCTACGCCAAGAAAACCGGCGACCAGAAGGGCTACCGCGAGGCT
CTGTGCAAGTGGATCGACTTTACCCGGGACTTCCTGAGCAAGTACACCAAGA
CCACCTCCATCGATCTGAGCAGCCTGCGGCCCAGCTCCCAGTACAAGGATCT
GGGCGAGTACTACGCCGAGCTGAACCCTCTGCTGTACCACATCAGCTTCCAG
CGGATCGCCGAAAAAGAAATCATGGACGCCGTGGAAACCGGCAAGCTGTAC
CTGTTCCAGATCTATAACAAGGACTTCGCCAAGGGCCACCACGGCAAGCCCA
ATCTGCACACCCTGTACTGGACCGGCCTGTTTAGCCCCGAGAATCTGGCCAA
GACCAGCATCAAGCTGAACGGCCAGGCCGAACTGTTTTACCGGCCCAAGAGC
CGGATGAAGCGGATGGCCCATAGACTGGGCGAGAAGATGCTGAACAAGAAA
CTGAAGGACCAGAAAACCCCTATCCCCGACACACTGTATCAGGAACTGTACG
ACTACGTGAACCACCGGCTGAGCCACGACCTGTCCGACGAAGCTAGAGCACT
GCTGCCCAACGTGATCACAAAAGAGGTGTCCCACGAGATCATCAAGGACCGG
CGGTTTACCTCCGATAAGTTCTTCTTCCACGTGCCCATCACCCTGAACTACCA
GGCCGCCAACAGCCCCAGCAAGTTCAACCAGAGAGTGAACGCCTACCTGAA
AGAGCACCCCGAGACACCCATCATTGGCATCGACAGAGGCGAGCGGAACCT
GATCTACATCACCGTGATCGACAGCACAGGCAAAATCCTGGAACAGAGAAG
CCTGAACACCATCCAGCAGTTCGACTACCAGAAGAAACTGGACAACCGGGA
AAAAGAACGGGTGGCCGCCAGACAGGCTTGGAGCGTCGTGGGCACCATTAA
GGACCTGAAGCAGGGCTACCTGAGCCAAGTGATTCACGAGATCGTGGACCTG
ATGATCCACTATCAGGCTGTGGTGGTGCTGGAAAACCTGAACTTCGGCTTCA
AGAGCAAGCGGACCGGAATCGCCGAGAAAGCCGTGTACCAGCAGTTTGAGA
AAATGCTGATCGACAAGCTGAATTGCCTGGTGCTGAAAGACTACCCCGCTGA
GAAAGTGGGAGGCGTGCTGAATCCCTACCAGCTGACCGACCAGTTCACCTCC
TTTGCCAAGATGGGAACCCAGAGCGGCTTCCTGTTCTACGTGCCAGCCCCCTA
CACCAGCAAGATCGACCCTCTGACCGGCTTCGTGGACCCCTTCGTGTGGAAA
ACCATCAAGAACCACGAGTCCCGGAAGCACTTCCTGGAAGGCTTTGACTTCC
TGCACTACGACGTGAAAACAGGCGATTTCATCCTGCACTTCAAGATGAATCG
GAATCTGTCCTTCCAGAGGGGCCTGCCCGGCTTCATGCCTGCCTGGGATATCG
TGTTCGAGAAGAATGAGACACAGTTCGACGCCAAGGGAACCCCCTTTATCGC
CGGCAAGAGGATCGTGCCTGTGATCGAGAACCACAGATTCACCGGCAGATAC
CGGGACCTGTACCCCGCCAACGAGCTGATTGCCCTGCTGGAAGAGAAGGGCA
TCGTGTTCCGGGACGGCAGCAACATCCTGCCCAAGCTGCTGGAAAATGACGA
CAGCCACGCCATCGATACCATGGTGGCACTGATCCGCAGCGTGCTGCAGATG
CGGAACAGCAATGCCGCCACCGGCGAGGACTACATCAATAGCCCAGTGCGG
GACCTGAACGGCGTGTGCTTCGACAGCAGATTCCAGAACCCCGAGTGGCCCA
TGGATGCCGACGCCAATGGCGCCTACCACATTGCCCTGAAGGGACAGCTGCT
GCTGAACCATCTGAAAGAGAGCAAAGACCTGAAACTGCAGAACGGCATCTC
CAACCAGGACTGGCTGGCCTATATCCAGGAACTGCGGAAC
C608S mutant Cas12a DNA sequence
SEQ ID NO. 17
ATGACCCAGTTCGAGGGCTTCACCAACCTGTACCAGGTGTCCAAGACCCTGA
GATTCGAGCTGATCCCCCAGGGCAAGACACTGAAGCACATCCAGGAACAGG
GCTTCATCGAAGAGGACAAGGCCCGGAACGACCACTACAAAGAGCTGAAGC
CCATCATCGACCGGATCTACAAGACCTACGCCGACCAGTGCCTGCAGCTGGT
GCAGCTGGACTGGGAGAATCTGAGCGCCGCCATCGACAGCTACCGGAAAGA
GAAAACCGAGGAAACCCGGAACGCCCTGATCGAGGAACAGGCCACCTACAG
AAACGCCATCCACGACTACTTCATCGGCCGGACCGACAACCTGACCGACGCC
ATCAACAAGCGGCACGCCGAGATCTATAAGGGCCTGTTCAAGGCCGAGCTGT
TCAACGGCAAGGTGCTGAAGCAGCTGGGCACCGTGACCACCACCGAGCACG
AAAACGCCCTGCTGCGGAGCTTCGACAAGTTCACCACCTACTTCAGCGGCTT
CTACGAGAACCGGAAGAACGTGTTCAGCGCCGAGGACATCAGCACCGCCATC
CCCCACAGAATCGTGCAGGACAACTTCCCCAAGTTCAAAGAGAACTGCCACA
TCTTCACCCGGCTGATCACCGCCGTGCCCAGCCTGAGAGAACACTTCGAGAA
CGTGAAGAAGGCCATCGGCATCTTCGTGTCCACCAGCATCGAGGAAGTGTTC
AGCTTCCCATTCTACAACCAGCTGCTGACCCAGACCCAGATCGACCTGTATA
ATCAGCTGCTGGGCGGCATCAGCAGAGAGGCCGGCACCGAGAAGATCAAGG
GCCTGAACGAAGTGCTGAACCTGGCCATCCAGAAGAACGACGAGACAGCCC
ACATCATTGCCAGCCTGCCCCACCGGTTCATCCCTCTGTTCAAGCAGATCCTG
AGCGACAGAAACACCCTGAGCTTCATCCTGGAAGAGTTCAAGTCCGATGAGG
AAGTGATCCAGAGCTTCTGCAAGTATAAGACCCTGCTGAGGAACGAGAATGT
GCTGGAAACCGCCGAGGCCCTGTTCAATGAGCTGAACAGCATCGACCTGACC
CACATCTTTATCAGCCACAAGAAGCTGGAAACAATCAGCAGCGCCCTGTGCG
ACCACTGGGACACACTGCGGAATGCCCTGTACGAGCGGCGGATCTCTGAGCT
GACCGGCAAGATCACCAAGAGCGCCAAAGAAAAGGTGCAGCGGAGCCTGAA
GCACGAGGATATCAACCTGCAGGAAATCATCAGCGCCGCTGGCAAAGAACT
GAGCGAGGCCTTTAAGCAGAAAACCAGCGAGATCCTGTCCCACGCCCACGCC
GCACTGGATCAGCCTCTGCCTACCACCCTGAAGAAGCAGGAAGAGAAAGAG
ATCCTGAAGTCCCAGCTGGACAGCCTGCTGGGCCTGTACCATCTGCTGGATTG
GTTCGCCGTGGACGAGAGCAACGAGGTGGACCCCGAGTTCTCCGCCAGACTG
ACAGGCATCAAACTGGAAATGGAACCCAGCCTGTCCTTCTACAACAAGGCCA
GAAACTACGCCACCAAGAAACCCTACAGCGTGGAAAAGTTTAAGCTGAACTT
CCAGATGCCCACCCTGGCCAGCGGCTGGGACGTGAACAAAGAGAAGAACAA
CGGCGCCATCCTGTTCGTGAAGAACGGACTGTACTACCTGGGCATCATGCCT
AAGCAGAAGGGCAGATACAAGGCCCTGTCCTTTGAGCCCACCGAAAAGACC
AGCGAGGGCTTTGACAAGATGTACTACGATTACTTCCCCGACGCCGCCAAGA
TGATCCCCAAGTCTAGCACCCAGCTGAAGGCCGTGACCGCCCACTTTCAGAC
CCACACCACCCCCATCCTGCTGAGCAACAACTTCATCGAGCCCCTGGAAATC
ACCAAAGAGATCTACGACCTGAACAACCCCGAGAAAGAGCCCAAGAAGTTC
CAGACCGCCTACGCCAAGAAAACCGGCGACCAGAAGGGCTACCGCGAGGCT
CTGTGCAAGTGGATCGACTTTACCCGGGACTTCCTGAGCAAGTACACCAAGA
CCACCTCCATCGATCTGAGCAGCCTGCGGCCCAGCTCCCAGTACAAGGATCT
GGGCGAGTACTACGCCGAGCTGAACCCTCTGCTGTACCACATCAGCTTCCAG
CGGATCGCCGAAAAAGAAATCATGGACGCCGTGGAAACCGGCAAGCTGTAC
CTGTTCCAGATCTATAACAAGGACTTCGCCAAGGGCCACCACGGCAAGCCCA
ATCTGCACACCCTGTACTGGACCGGCCTGTTTAGCCCCGAGAATCTGGCCAA
GACCAGCATCAAGCTGAACGGCCAGGCCGAACTGTTTTACCGGCCCAAGAGC
CGGATGAAGCGGATGGCCCATAGACTGGGCGAGAAGATGCTGAACAAGAAA
CTGAAGGACCAGAAAACCCCTATCCCCGACACACTGTATCAGGAACTGTACG
ACTACGTGAACCACCGGCTGAGCCACGACCTGTCCGACGAAGCTAGAGCACT
GCTGCCCAACGTGATCACAAAAGAGGTGTCCCACGAGATCATCAAGGACCGG
CGGTTTACCTCCGATAAGTTCTTCTTCCACGTGCCCATCACCCTGAACTACCA
GGCCGCCAACAGCCCCAGCAAGTTCAACCAGAGAGTGAACGCCTACCTGAA
AGAGCACCCCGAGACACCCATCATTGGCATCGACAGAGGCGAGCGGAACCT
GATCTACATCACCGTGATCGACAGCACAGGCAAAATCCTGGAACAGAGAAG
CCTGAACACCATCCAGCAGTTCGACTACCAGAAGAAACTGGACAACCGGGA
AAAAGAACGGGTGGCCGCCAGACAGGCTTGGAGCGTCGTGGGCACCATTAA
GGACCTGAAGCAGGGCTACCTGAGCCAAGTGATTCACGAGATCGTGGACCTG
ATGATCCACTATCAGGCTGTGGTGGTGCTGGAAAACCTGAACTTCGGCTTCA
AGAGCAAGCGGACCGGAATCGCCGAGAAAGCCGTGTACCAGCAGTTTGAGA
AAATGCTGATCGACAAGCTGAATTGCCTGGTGCTGAAAGACTACCCCGCTGA
GAAAGTGGGAGGCGTGCTGAATCCCTACCAGCTGACCGACCAGTTCACCTCC
TTTGCCAAGATGGGAACCCAGAGCGGCTTCCTGTTCTACGTGCCAGCCCCCTA
CACCAGCAAGATCGACCCTCTGACCGGCTTCGTGGACCCCTTCGTGTGGAAA
ACCATCAAGAACCACGAGTCCCGGAAGCACTTCCTGGAAGGCTTTGACTTCC
TGCACTACGACGTGAAAACAGGCGATTTCATCCTGCACTTCAAGATGAATCG
GAATCTGTCCTTCCAGAGGGGCCTGCCCGGCTTCATGCCTGCCTGGGATATCG
TGTTCGAGAAGAATGAGACACAGTTCGACGCCAAGGGAACCCCCTTTATCGC
CGGCAAGAGGATCGTGCCTGTGATCGAGAACCACAGATTCACCGGCAGATAC
CGGGACCTGTACCCCGCCAACGAGCTGATTGCCCTGCTGGAAGAGAAGGGCA
TCGTGTTCCGGGACGGCAGCAACATCCTGCCCAAGCTGCTGGAAAATGACGA
CAGCCACGCCATCGATACCATGGTGGCACTGATCCGCAGCGTGCTGCAGATG
CGGAACAGCAATGCCGCCACCGGCGAGGACTACATCAATAGCCCAGTGCGG
GACCTGAACGGCGTGTGCTTCGACAGCAGATTCCAGAACCCCGAGTGGCCCA
TGGATGCCGACGCCAATGGCGCCTACCACATTGCCCTGAAGGGACAGCTGCT
GCTGAACCATCTGAAAGAGAGCAAAGACCTGAAACTGCAGAACGGCATCTC
CAACCAGGACTGGCTGGCCTATATCCAGGAACTGCGGAAC
C674S mutant Cas12a DNA sequence
SEQ ID NO. 18
ATGACCCAGTTCGAGGGCTTCACCAACCTGTACCAGGTGTCCAAGACCCTGA
GATTCGAGCTGATCCCCCAGGGCAAGACACTGAAGCACATCCAGGAACAGG
GCTTCATCGAAGAGGACAAGGCCCGGAACGACCACTACAAAGAGCTGAAGC
CCATCATCGACCGGATCTACAAGACCTACGCCGACCAGTGCCTGCAGCTGGT
GCAGCTGGACTGGGAGAATCTGAGCGCCGCCATCGACAGCTACCGGAAAGA
GAAAACCGAGGAAACCCGGAACGCCCTGATCGAGGAACAGGCCACCTACAG
AAACGCCATCCACGACTACTTCATCGGCCGGACCGACAACCTGACCGACGCC
ATCAACAAGCGGCACGCCGAGATCTATAAGGGCCTGTTCAAGGCCGAGCTGT
TCAACGGCAAGGTGCTGAAGCAGCTGGGCACCGTGACCACCACCGAGCACG
AAAACGCCCTGCTGCGGAGCTTCGACAAGTTCACCACCTACTTCAGCGGCTT
CTACGAGAACCGGAAGAACGTGTTCAGCGCCGAGGACATCAGCACCGCCATC
CCCCACAGAATCGTGCAGGACAACTTCCCCAAGTTCAAAGAGAACTGCCACA
TCTTCACCCGGCTGATCACCGCCGTGCCCAGCCTGAGAGAACACTTCGAGAA
CGTGAAGAAGGCCATCGGCATCTTCGTGTCCACCAGCATCGAGGAAGTGTTC
AGCTTCCCATTCTACAACCAGCTGCTGACCCAGACCCAGATCGACCTGTATA
ATCAGCTGCTGGGCGGCATCAGCAGAGAGGCCGGCACCGAGAAGATCAAGG
GCCTGAACGAAGTGCTGAACCTGGCCATCCAGAAGAACGACGAGACAGCCC
ACATCATTGCCAGCCTGCCCCACCGGTTCATCCCTCTGTTCAAGCAGATCCTG
AGCGACAGAAACACCCTGAGCTTCATCCTGGAAGAGTTCAAGTCCGATGAGG
AAGTGATCCAGAGCTTCTGCAAGTATAAGACCCTGCTGAGGAACGAGAATGT
GCTGGAAACCGCCGAGGCCCTGTTCAATGAGCTGAACAGCATCGACCTGACC
CACATCTTTATCAGCCACAAGAAGCTGGAAACAATCAGCAGCGCCCTGTGCG
ACCACTGGGACACACTGCGGAATGCCCTGTACGAGCGGCGGATCTCTGAGCT
GACCGGCAAGATCACCAAGAGCGCCAAAGAAAAGGTGCAGCGGAGCCTGAA
GCACGAGGATATCAACCTGCAGGAAATCATCAGCGCCGCTGGCAAAGAACT
GAGCGAGGCCTTTAAGCAGAAAACCAGCGAGATCCTGTCCCACGCCCACGCC
GCACTGGATCAGCCTCTGCCTACCACCCTGAAGAAGCAGGAAGAGAAAGAG
ATCCTGAAGTCCCAGCTGGACAGCCTGCTGGGCCTGTACCATCTGCTGGATTG
GTTCGCCGTGGACGAGAGCAACGAGGTGGACCCCGAGTTCTCCGCCAGACTG
ACAGGCATCAAACTGGAAATGGAACCCAGCCTGTCCTTCTACAACAAGGCCA
GAAACTACGCCACCAAGAAACCCTACAGCGTGGAAAAGTTTAAGCTGAACTT
CCAGATGCCCACCCTGGCCAGCGGCTGGGACGTGAACAAAGAGAAGAACAA
CGGCGCCATCCTGTTCGTGAAGAACGGACTGTACTACCTGGGCATCATGCCT
AAGCAGAAGGGCAGATACAAGGCCCTGTCCTTTGAGCCCACCGAAAAGACC
AGCGAGGGCTTTGACAAGATGTACTACGATTACTTCCCCGACGCCGCCAAGA
TGATCCCCAAGTGCAGCACCCAGCTGAAGGCCGTGACCGCCCACTTTCAGAC
CCACACCACCCCCATCCTGCTGAGCAACAACTTCATCGAGCCCCTGGAAATC
ACCAAAGAGATCTACGACCTGAACAACCCCGAGAAAGAGCCCAAGAAGTTC
CAGACCGCCTACGCCAAGAAAACCGGCGACCAGAAGGGCTACCGCGAGGCT
CTGTCTAAGTGGATCGACTTTACCCGGGACTTCCTGAGCAAGTACACCAAGA
CCACCTCCATCGATCTGAGCAGCCTGCGGCCCAGCTCCCAGTACAAGGATCT
GGGCGAGTACTACGCCGAGCTGAACCCTCTGCTGTACCACATCAGCTTCCAG
CGGATCGCCGAAAAAGAAATCATGGACGCCGTGGAAACCGGCAAGCTGTAC
CTGTTCCAGATCTATAACAAGGACTTCGCCAAGGGCCACCACGGCAAGCCCA
ATCTGCACACCCTGTACTGGACCGGCCTGTTTAGCCCCGAGAATCTGGCCAA
GACCAGCATCAAGCTGAACGGCCAGGCCGAACTGTTTTACCGGCCCAAGAGC
CGGATGAAGCGGATGGCCCATAGACTGGGCGAGAAGATGCTGAACAAGAAA
CTGAAGGACCAGAAAACCCCTATCCCCGACACACTGTATCAGGAACTGTACG
ACTACGTGAACCACCGGCTGAGCCACGACCTGTCCGACGAAGCTAGAGCACT
GCTGCCCAACGTGATCACAAAAGAGGTGTCCCACGAGATCATCAAGGACCGG
CGGTTTACCTCCGATAAGTTCTTCTTCCACGTGCCCATCACCCTGAACTACCA
GGCCGCCAACAGCCCCAGCAAGTTCAACCAGAGAGTGAACGCCTACCTGAA
AGAGCACCCCGAGACACCCATCATTGGCATCGACAGAGGCGAGCGGAACCT
GATCTACATCACCGTGATCGACAGCACAGGCAAAATCCTGGAACAGAGAAG
CCTGAACACCATCCAGCAGTTCGACTACCAGAAGAAACTGGACAACCGGGA
AAAAGAACGGGTGGCCGCCAGACAGGCTTGGAGCGTCGTGGGCACCATTAA
GGACCTGAAGCAGGGCTACCTGAGCCAAGTGATTCACGAGATCGTGGACCTG
ATGATCCACTATCAGGCTGTGGTGGTGCTGGAAAACCTGAACTTCGGCTTCA
AGAGCAAGCGGACCGGAATCGCCGAGAAAGCCGTGTACCAGCAGTTTGAGA
AAATGCTGATCGACAAGCTGAATTGCCTGGTGCTGAAAGACTACCCCGCTGA
GAAAGTGGGAGGCGTGCTGAATCCCTACCAGCTGACCGACCAGTTCACCTCC
TTTGCCAAGATGGGAACCCAGAGCGGCTTCCTGTTCTACGTGCCAGCCCCCTA
CACCAGCAAGATCGACCCTCTGACCGGCTTCGTGGACCCCTTCGTGTGGAAA
ACCATCAAGAACCACGAGTCCCGGAAGCACTTCCTGGAAGGCTTTGACTTCC
TGCACTACGACGTGAAAACAGGCGATTTCATCCTGCACTTCAAGATGAATCG
GAATCTGTCCTTCCAGAGGGGCCTGCCCGGCTTCATGCCTGCCTGGGATATCG
TGTTCGAGAAGAATGAGACACAGTTCGACGCCAAGGGAACCCCCTTTATCGC
CGGCAAGAGGATCGTGCCTGTGATCGAGAACCACAGATTCACCGGCAGATAC
CGGGACCTGTACCCCGCCAACGAGCTGATTGCCCTGCTGGAAGAGAAGGGCA
TCGTGTTCCGGGACGGCAGCAACATCCTGCCCAAGCTGCTGGAAAATGACGA
CAGCCACGCCATCGATACCATGGTGGCACTGATCCGCAGCGTGCTGCAGATG
CGGAACAGCAATGCCGCCACCGGCGAGGACTACATCAATAGCCCAGTGCGG
GACCTGAACGGCGTGTGCTTCGACAGCAGATTCCAGAACCCCGAGTGGCCCA
TGGATGCCGACGCCAATGGCGCCTACCACATTGCCCTGAAGGGACAGCTGCT
GCTGAACCATCTGAAAGAGAGCAAAGACCTGAAACTGCAGAACGGCATCTC
CAACCAGGACTGGCTGGCCTATATCCAGGAACTGCGGAAC
C1025S mutant Cas12a DNA sequence
SEQ ID NO. 19
ATGACCCAGTTCGAGGGCTTCACCAACCTGTACCAGGTGTCCAAGACCCTGA
GATTCGAGCTGATCCCCCAGGGCAAGACACTGAAGCACATCCAGGAACAGG
GCTTCATCGAAGAGGACAAGGCCCGGAACGACCACTACAAAGAGCTGAAGC
CCATCATCGACCGGATCTACAAGACCTACGCCGACCAGTGCCTGCAGCTGGT
GCAGCTGGACTGGGAGAATCTGAGCGCCGCCATCGACAGCTACCGGAAAGA
GAAAACCGAGGAAACCCGGAACGCCCTGATCGAGGAACAGGCCACCTACAG
AAACGCCATCCACGACTACTTCATCGGCCGGACCGACAACCTGACCGACGCC
ATCAACAAGCGGCACGCCGAGATCTATAAGGGCCTGTTCAAGGCCGAGCTGT
TCAACGGCAAGGTGCTGAAGCAGCTGGGCACCGTGACCACCACCGAGCACG
AAAACGCCCTGCTGCGGAGCTTCGACAAGTTCACCACCTACTTCAGCGGCTT
CTACGAGAACCGGAAGAACGTGTTCAGCGCCGAGGACATCAGCACCGCCATC
CCCCACAGAATCGTGCAGGACAACTTCCCCAAGTTCAAAGAGAACTGCCACA
TCTTCACCCGGCTGATCACCGCCGTGCCCAGCCTGAGAGAACACTTCGAGAA
CGTGAAGAAGGCCATCGGCATCTTCGTGTCCACCAGCATCGAGGAAGTGTTC
AGCTTCCCATTCTACAACCAGCTGCTGACCCAGACCCAGATCGACCTGTATA
ATCAGCTGCTGGGCGGCATCAGCAGAGAGGCCGGCACCGAGAAGATCAAGG
GCCTGAACGAAGTGCTGAACCTGGCCATCCAGAAGAACGACGAGACAGCCC
ACATCATTGCCAGCCTGCCCCACCGGTTCATCCCTCTGTTCAAGCAGATCCTG
AGCGACAGAAACACCCTGAGCTTCATCCTGGAAGAGTTCAAGTCCGATGAGG
AAGTGATCCAGAGCTTCTGCAAGTATAAGACCCTGCTGAGGAACGAGAATGT
GCTGGAAACCGCCGAGGCCCTGTTCAATGAGCTGAACAGCATCGACCTGACC
CACATCTTTATCAGCCACAAGAAGCTGGAAACAATCAGCAGCGCCCTGTGCG
ACCACTGGGACACACTGCGGAATGCCCTGTACGAGCGGCGGATCTCTGAGCT
GACCGGCAAGATCACCAAGAGCGCCAAAGAAAAGGTGCAGCGGAGCCTGAA
GCACGAGGATATCAACCTGCAGGAAATCATCAGCGCCGCTGGCAAAGAACT
GAGCGAGGCCTTTAAGCAGAAAACCAGCGAGATCCTGTCCCACGCCCACGCC
GCACTGGATCAGCCTCTGCCTACCACCCTGAAGAAGCAGGAAGAGAAAGAG
ATCCTGAAGTCCCAGCTGGACAGCCTGCTGGGCCTGTACCATCTGCTGGATTG
GTTCGCCGTGGACGAGAGCAACGAGGTGGACCCCGAGTTCTCCGCCAGACTG
ACAGGCATCAAACTGGAAATGGAACCCAGCCTGTCCTTCTACAACAAGGCCA
GAAACTACGCCACCAAGAAACCCTACAGCGTGGAAAAGTTTAAGCTGAACTT
CCAGATGCCCACCCTGGCCAGCGGCTGGGACGTGAACAAAGAGAAGAACAA
CGGCGCCATCCTGTTCGTGAAGAACGGACTGTACTACCTGGGCATCATGCCT
AAGCAGAAGGGCAGATACAAGGCCCTGTCCTTTGAGCCCACCGAAAAGACC
AGCGAGGGCTTTGACAAGATGTACTACGATTACTTCCCCGACGCCGCCAAGA
TGATCCCCAAGTGCAGCACCCAGCTGAAGGCCGTGACCGCCCACTTTCAGAC
CCACACCACCCCCATCCTGCTGAGCAACAACTTCATCGAGCCCCTGGAAATC
ACCAAAGAGATCTACGACCTGAACAACCCCGAGAAAGAGCCCAAGAAGTTC
CAGACCGCCTACGCCAAGAAAACCGGCGACCAGAAGGGCTACCGCGAGGCT
CTGTGCAAGTGGATCGACTTTACCCGGGACTTCCTGAGCAAGTACACCAAGA
CCACCTCCATCGATCTGAGCAGCCTGCGGCCCAGCTCCCAGTACAAGGATCT
GGGCGAGTACTACGCCGAGCTGAACCCTCTGCTGTACCACATCAGCTTCCAG
CGGATCGCCGAAAAAGAAATCATGGACGCCGTGGAAACCGGCAAGCTGTAC
CTGTTCCAGATCTATAACAAGGACTTCGCCAAGGGCCACCACGGCAAGCCCA
ATCTGCACACCCTGTACTGGACCGGCCTGTTTAGCCCCGAGAATCTGGCCAA
GACCAGCATCAAGCTGAACGGCCAGGCCGAACTGTTTTACCGGCCCAAGAGC
CGGATGAAGCGGATGGCCCATAGACTGGGCGAGAAGATGCTGAACAAGAAA
CTGAAGGACCAGAAAACCCCTATCCCCGACACACTGTATCAGGAACTGTACG
ACTACGTGAACCACCGGCTGAGCCACGACCTGTCCGACGAAGCTAGAGCACT
GCTGCCCAACGTGATCACAAAAGAGGTGTCCCACGAGATCATCAAGGACCGG
CGGTTTACCTCCGATAAGTTCTTCTTCCACGTGCCCATCACCCTGAACTACCA
GGCCGCCAACAGCCCCAGCAAGTTCAACCAGAGAGTGAACGCCTACCTGAA
AGAGCACCCCGAGACACCCATCATTGGCATCGACAGAGGCGAGCGGAACCT
GATCTACATCACCGTGATCGACAGCACAGGCAAAATCCTGGAACAGAGAAG
CCTGAACACCATCCAGCAGTTCGACTACCAGAAGAAACTGGACAACCGGGA
AAAAGAACGGGTGGCCGCCAGACAGGCTTGGAGCGTCGTGGGCACCATTAA
GGACCTGAAGCAGGGCTACCTGAGCCAAGTGATTCACGAGATCGTGGACCTG
ATGATCCACTATCAGGCTGTGGTGGTGCTGGAAAACCTGAACTTCGGCTTCA
AGAGCAAGCGGACCGGAATCGCCGAGAAAGCCGTGTACCAGCAGTTTGAGA
AAATGCTGATCGACAAGCTGAATTCTCTGGTGCTGAAAGACTACCCCGCTGA
GAAAGTGGGAGGCGTGCTGAATCCCTACCAGCTGACCGACCAGTTCACCTCC
TTTGCCAAGATGGGAACCCAGAGCGGCTTCCTGTTCTACGTGCCAGCCCCCTA
CACCAGCAAGATCGACCCTCTGACCGGCTTCGTGGACCCCTTCGTGTGGAAA
ACCATCAAGAACCACGAGTCCCGGAAGCACTTCCTGGAAGGCTTTGACTTCC
TGCACTACGACGTGAAAACAGGCGATTTCATCCTGCACTTCAAGATGAATCG
GAATCTGTCCTTCCAGAGGGGCCTGCCCGGCTTCATGCCTGCCTGGGATATCG
TGTTCGAGAAGAATGAGACACAGTTCGACGCCAAGGGAACCCCCTTTATCGC
CGGCAAGAGGATCGTGCCTGTGATCGAGAACCACAGATTCACCGGCAGATAC
CGGGACCTGTACCCCGCCAACGAGCTGATTGCCCTGCTGGAAGAGAAGGGCA
TCGTGTTCCGGGACGGCAGCAACATCCTGCCCAAGCTGCTGGAAAATGACGA
CAGCCACGCCATCGATACCATGGTGGCACTGATCCGCAGCGTGCTGCAGATG
CGGAACAGCAATGCCGCCACCGGCGAGGACTACATCAATAGCCCAGTGCGG
GACCTGAACGGCGTGTGCTTCGACAGCAGATTCCAGAACCCCGAGTGGCCCA
TGGATGCCGACGCCAATGGCGCCTACCACATTGCCCTGAAGGGACAGCTGCT
GCTGAACCATCTGAAAGAGAGCAAAGACCTGAAACTGCAGAACGGCATCTC
CAACCAGGACTGGCTGGCCTATATCCAGGAACTGCGGAAC
C1248S mutant Cas12a DNA sequence
SEQ ID NO. 20
ATGACCCAGTTCGAGGGCTTCACCAACCTGTACCAGGTGTCCAAGACCCTGA
GATTCGAGCTGATCCCCCAGGGCAAGACACTGAAGCACATCCAGGAACAGG
GCTTCATCGAAGAGGACAAGGCCCGGAACGACCACTACAAAGAGCTGAAGC
CCATCATCGACCGGATCTACAAGACCTACGCCGACCAGTGCCTGCAGCTGGT
GCAGCTGGACTGGGAGAATCTGAGCGCCGCCATCGACAGCTACCGGAAAGA
GAAAACCGAGGAAACCCGGAACGCCCTGATCGAGGAACAGGCCACCTACAG
AAACGCCATCCACGACTACTTCATCGGCCGGACCGACAACCTGACCGACGCC
ATCAACAAGCGGCACGCCGAGATCTATAAGGGCCTGTTCAAGGCCGAGCTGT
TCAACGGCAAGGTGCTGAAGCAGCTGGGCACCGTGACCACCACCGAGCACG
AAAACGCCCTGCTGCGGAGCTTCGACAAGTTCACCACCTACTTCAGCGGCTT
CTACGAGAACCGGAAGAACGTGTTCAGCGCCGAGGACATCAGCACCGCCATC
CCCCACAGAATCGTGCAGGACAACTTCCCCAAGTTCAAAGAGAACTGCCACA
TCTTCACCCGGCTGATCACCGCCGTGCCCAGCCTGAGAGAACACTTCGAGAA
CGTGAAGAAGGCCATCGGCATCTTCGTGTCCACCAGCATCGAGGAAGTGTTC
AGCTTCCCATTCTACAACCAGCTGCTGACCCAGACCCAGATCGACCTGTATA
ATCAGCTGCTGGGCGGCATCAGCAGAGAGGCCGGCACCGAGAAGATCAAGG
GCCTGAACGAAGTGCTGAACCTGGCCATCCAGAAGAACGACGAGACAGCCC
ACATCATTGCCAGCCTGCCCCACCGGTTCATCCCTCTGTTCAAGCAGATCCTG
AGCGACAGAAACACCCTGAGCTTCATCCTGGAAGAGTTCAAGTCCGATGAGG
AAGTGATCCAGAGCTTCTGCAAGTATAAGACCCTGCTGAGGAACGAGAATGT
GCTGGAAACCGCCGAGGCCCTGTTCAATGAGCTGAACAGCATCGACCTGACC
CACATCTTTATCAGCCACAAGAAGCTGGAAACAATCAGCAGCGCCCTGTGCG
ACCACTGGGACACACTGCGGAATGCCCTGTACGAGCGGCGGATCTCTGAGCT
GACCGGCAAGATCACCAAGAGCGCCAAAGAAAAGGTGCAGCGGAGCCTGAA
GCACGAGGATATCAACCTGCAGGAAATCATCAGCGCCGCTGGCAAAGAACT
GAGCGAGGCCTTTAAGCAGAAAACCAGCGAGATCCTGTCCCACGCCCACGCC
GCACTGGATCAGCCTCTGCCTACCACCCTGAAGAAGCAGGAAGAGAAAGAG
ATCCTGAAGTCCCAGCTGGACAGCCTGCTGGGCCTGTACCATCTGCTGGATTG
GTTCGCCGTGGACGAGAGCAACGAGGTGGACCCCGAGTTCTCCGCCAGACTG
ACAGGCATCAAACTGGAAATGGAACCCAGCCTGTCCTTCTACAACAAGGCCA
GAAACTACGCCACCAAGAAACCCTACAGCGTGGAAAAGTTTAAGCTGAACTT
CCAGATGCCCACCCTGGCCAGCGGCTGGGACGTGAACAAAGAGAAGAACAA
CGGCGCCATCCTGTTCGTGAAGAACGGACTGTACTACCTGGGCATCATGCCT
AAGCAGAAGGGCAGATACAAGGCCCTGTCCTTTGAGCCCACCGAAAAGACC
AGCGAGGGCTTTGACAAGATGTACTACGATTACTTCCCCGACGCCGCCAAGA
TGATCCCCAAGTGCAGCACCCAGCTGAAGGCCGTGACCGCCCACTTTCAGAC
CCACACCACCCCCATCCTGCTGAGCAACAACTTCATCGAGCCCCTGGAAATC
ACCAAAGAGATCTACGACCTGAACAACCCCGAGAAAGAGCCCAAGAAGTTC
CAGACCGCCTACGCCAAGAAAACCGGCGACCAGAAGGGCTACCGCGAGGCT
CTGTGCAAGTGGATCGACTTTACCCGGGACTTCCTGAGCAAGTACACCAAGA
CCACCTCCATCGATCTGAGCAGCCTGCGGCCCAGCTCCCAGTACAAGGATCT
GGGCGAGTACTACGCCGAGCTGAACCCTCTGCTGTACCACATCAGCTTCCAG
CGGATCGCCGAAAAAGAAATCATGGACGCCGTGGAAACCGGCAAGCTGTAC
CTGTTCCAGATCTATAACAAGGACTTCGCCAAGGGCCACCACGGCAAGCCCA
ATCTGCACACCCTGTACTGGACCGGCCTGTTTAGCCCCGAGAATCTGGCCAA
GACCAGCATCAAGCTGAACGGCCAGGCCGAACTGTTTTACCGGCCCAAGAGC
CGGATGAAGCGGATGGCCCATAGACTGGGCGAGAAGATGCTGAACAAGAAA
CTGAAGGACCAGAAAACCCCTATCCCCGACACACTGTATCAGGAACTGTACG
ACTACGTGAACCACCGGCTGAGCCACGACCTGTCCGACGAAGCTAGAGCACT
GCTGCCCAACGTGATCACAAAAGAGGTGTCCCACGAGATCATCAAGGACCGG
CGGTTTACCTCCGATAAGTTCTTCTTCCACGTGCCCATCACCCTGAACTACCA
GGCCGCCAACAGCCCCAGCAAGTTCAACCAGAGAGTGAACGCCTACCTGAA
AGAGCACCCCGAGACACCCATCATTGGCATCGACAGAGGCGAGCGGAACCT
GATCTACATCACCGTGATCGACAGCACAGGCAAAATCCTGGAACAGAGAAG
CCTGAACACCATCCAGCAGTTCGACTACCAGAAGAAACTGGACAACCGGGA
AAAAGAACGGGTGGCCGCCAGACAGGCTTGGAGCGTCGTGGGCACCATTAA
GGACCTGAAGCAGGGCTACCTGAGCCAAGTGATTCACGAGATCGTGGACCTG
ATGATCCACTATCAGGCTGTGGTGGTGCTGGAAAACCTGAACTTCGGCTTCA
AGAGCAAGCGGACCGGAATCGCCGAGAAAGCCGTGTACCAGCAGTTTGAGA
AAATGCTGATCGACAAGCTGAATTGCCTGGTGCTGAAAGACTACCCCGCTGA
GAAAGTGGGAGGCGTGCTGAATCCCTACCAGCTGACCGACCAGTTCACCTCC
TTTGCCAAGATGGGAACCCAGAGCGGCTTCCTGTTCTACGTGCCAGCCCCCTA
CACCAGCAAGATCGACCCTCTGACCGGCTTCGTGGACCCCTTCGTGTGGAAA
ACCATCAAGAACCACGAGTCCCGGAAGCACTTCCTGGAAGGCTTTGACTTCC
TGCACTACGACGTGAAAACAGGCGATTTCATCCTGCACTTCAAGATGAATCG
GAATCTGTCCTTCCAGAGGGGCCTGCCCGGCTTCATGCCTGCCTGGGATATCG
TGTTCGAGAAGAATGAGACACAGTTCGACGCCAAGGGAACCCCCTTTATCGC
CGGCAAGAGGATCGTGCCTGTGATCGAGAACCACAGATTCACCGGCAGATAC
CGGGACCTGTACCCCGCCAACGAGCTGATTGCCCTGCTGGAAGAGAAGGGCA
TCGTGTTCCGGGACGGCAGCAACATCCTGCCCAAGCTGCTGGAAAATGACGA
CAGCCACGCCATCGATACCATGGTGGCACTGATCCGCAGCGTGCTGCAGATG
CGGAACAGCAATGCCGCCACCGGCGAGGACTACATCAATAGCCCAGTGCGG
GACCTGAACGGCGTGTCTTTCGACAGCAGATTCCAGAACCCCGAGTGGCCCA
TGGATGCCGACGCCAATGGCGCCTACCACATTGCCCTGAAGGGACAGCTGCT
GCTGAACCATCTGAAAGAGAGCAAAGACCTGAAACTGCAGAACGGCATCTC
CAACCAGGACTGGCTGGCCTATATCCAGGAACTGCGGAAC
C205S, C379S, C674S and C1248S mutant Cas12a DNA sequence
SEQ ID NO. 21
ATGACCCAGTTCGAGGGCTTCACCAACCTGTACCAGGTGTCCAAGACCCTGA
GATTCGAGCTGATCCCCCAGGGCAAGACACTGAAGCACATCCAGGAACAGG
GCTTCATCGAAGAGGACAAGGCCCGGAACGACCACTACAAAGAGCTGAAGC
CCATCATCGACCGGATCTACAAGACCTACGCCGACCAGTGCCTGCAGCTGGT
GCAGCTGGACTGGGAGAATCTGAGCGCCGCCATCGACAGCTACCGGAAAGA
GAAAACCGAGGAAACCCGGAACGCCCTGATCGAGGAACAGGCCACCTACAG
AAACGCCATCCACGACTACTTCATCGGCCGGACCGACAACCTGACCGACGCC
ATCAACAAGCGGCACGCCGAGATCTATAAGGGCCTGTTCAAGGCCGAGCTGT
TCAACGGCAAGGTGCTGAAGCAGCTGGGCACCGTGACCACCACCGAGCACG
AAAACGCCCTGCTGCGGAGCTTCGACAAGTTCACCACCTACTTCAGCGGCTT
CTACGAGAACCGGAAGAACGTGTTCAGCGCCGAGGACATCAGCACCGCCATC
CCCCACAGAATCGTGCAGGACAACTTCCCCAAGTTCAAAGAGAACTCTCACA
TCTTCACCCGGCTGATCACCGCCGTGCCCAGCCTGAGAGAACACTTCGAGAA
CGTGAAGAAGGCCATCGGCATCTTCGTGTCCACCAGCATCGAGGAAGTGTTC
AGCTTCCCATTCTACAACCAGCTGCTGACCCAGACCCAGATCGACCTGTATA
ATCAGCTGCTGGGCGGCATCAGCAGAGAGGCCGGCACCGAGAAGATCAAGG
GCCTGAACGAAGTGCTGAACCTGGCCATCCAGAAGAACGACGAGACAGCCC
ACATCATTGCCAGCCTGCCCCACCGGTTCATCCCTCTGTTCAAGCAGATCCTG
AGCGACAGAAACACCCTGAGCTTCATCCTGGAAGAGTTCAAGTCCGATGAGG
AAGTGATCCAGAGCTTCTGCAAGTATAAGACCCTGCTGAGGAACGAGAATGT
GCTGGAAACCGCCGAGGCCCTGTTCAATGAGCTGAACAGCATCGACCTGACC
CACATCTTTATCAGCCACAAGAAGCTGGAAACAATCAGCAGCGCCCTGTCTG
ACCACTGGGACACACTGCGGAATGCCCTGTACGAGCGGCGGATCTCTGAGCT
GACCGGCAAGATCACCAAGAGCGCCAAAGAAAAGGTGCAGCGGAGCCTGAA
GCACGAGGATATCAACCTGCAGGAAATCATCAGCGCCGCTGGCAAAGAACT
GAGCGAGGCCTTTAAGCAGAAAACCAGCGAGATCCTGTCCCACGCCCACGCC
GCACTGGATCAGCCTCTGCCTACCACCCTGAAGAAGCAGGAAGAGAAAGAG
ATCCTGAAGTCCCAGCTGGACAGCCTGCTGGGCCTGTACCATCTGCTGGATTG
GTTCGCCGTGGACGAGAGCAACGAGGTGGACCCCGAGTTCTCCGCCAGACTG
ACAGGCATCAAACTGGAAATGGAACCCAGCCTGTCCTTCTACAACAAGGCCA
GAAACTACGCCACCAAGAAACCCTACAGCGTGGAAAAGTTTAAGCTGAACTT
CCAGATGCCCACCCTGGCCAGCGGCTGGGACGTGAACAAAGAGAAGAACAA
CGGCGCCATCCTGTTCGTGAAGAACGGACTGTACTACCTGGGCATCATGCCT
AAGCAGAAGGGCAGATACAAGGCCCTGTCCTTTGAGCCCACCGAAAAGACC
AGCGAGGGCTTTGACAAGATGTACTACGATTACTTCCCCGACGCCGCCAAGA
TGATCCCCAAGTGCAGCACCCAGCTGAAGGCCGTGACCGCCCACTTTCAGAC
CCACACCACCCCCATCCTGCTGAGCAACAACTTCATCGAGCCCCTGGAAATC
ACCAAAGAGATCTACGACCTGAACAACCCCGAGAAAGAGCCCAAGAAGTTC
CAGACCGCCTACGCCAAGAAAACCGGCGACCAGAAGGGCTACCGCGAGGCT
CTGTCTAAGTGGATCGACTTTACCCGGGACTTCCTGAGCAAGTACACCAAGA
CCACCTCCATCGATCTGAGCAGCCTGCGGCCCAGCTCCCAGTACAAGGATCT
GGGCGAGTACTACGCCGAGCTGAACCCTCTGCTGTACCACATCAGCTTCCAG
CGGATCGCCGAAAAAGAAATCATGGACGCCGTGGAAACCGGCAAGCTGTAC
CTGTTCCAGATCTATAACAAGGACTTCGCCAAGGGCCACCACGGCAAGCCCA
ATCTGCACACCCTGTACTGGACCGGCCTGTTTAGCCCCGAGAATCTGGCCAA
GACCAGCATCAAGCTGAACGGCCAGGCCGAACTGTTTTACCGGCCCAAGAGC
CGGATGAAGCGGATGGCCCATAGACTGGGCGAGAAGATGCTGAACAAGAAA
CTGAAGGACCAGAAAACCCCTATCCCCGACACACTGTATCAGGAACTGTACG
ACTACGTGAACCACCGGCTGAGCCACGACCTGTCCGACGAAGCTAGAGCACT
GCTGCCCAACGTGATCACAAAAGAGGTGTCCCACGAGATCATCAAGGACCGG
CGGTTTACCTCCGATAAGTTCTTCTTCCACGTGCCCATCACCCTGAACTACCA
GGCCGCCAACAGCCCCAGCAAGTTCAACCAGAGAGTGAACGCCTACCTGAA
AGAGCACCCCGAGACACCCATCATTGGCATCGACAGAGGCGAGCGGAACCT
GATCTACATCACCGTGATCGACAGCACAGGCAAAATCCTGGAACAGAGAAG
CCTGAACACCATCCAGCAGTTCGACTACCAGAAGAAACTGGACAACCGGGA
AAAAGAACGGGTGGCCGCCAGACAGGCTTGGAGCGTCGTGGGCACCATTAA
GGACCTGAAGCAGGGCTACCTGAGCCAAGTGATTCACGAGATCGTGGACCTG
ATGATCCACTATCAGGCTGTGGTGGTGCTGGAAAACCTGAACTTCGGCTTCA
AGAGCAAGCGGACCGGAATCGCCGAGAAAGCCGTGTACCAGCAGTTTGAGA
AAATGCTGATCGACAAGCTGAATTGCCTGGTGCTGAAAGACTACCCCGCTGA
GAAAGTGGGAGGCGTGCTGAATCCCTACCAGCTGACCGACCAGTTCACCTCC
TTTGCCAAGATGGGAACCCAGAGCGGCTTCCTGTTCTACGTGCCAGCCCCCTA
CACCAGCAAGATCGACCCTCTGACCGGCTTCGTGGACCCCTTCGTGTGGAAA
ACCATCAAGAACCACGAGTCCCGGAAGCACTTCCTGGAAGGCTTTGACTTCC
TGCACTACGACGTGAAAACAGGCGATTTCATCCTGCACTTCAAGATGAATCG
GAATCTGTCCTTCCAGAGGGGCCTGCCCGGCTTCATGCCTGCCTGGGATATCG
TGTTCGAGAAGAATGAGACACAGTTCGACGCCAAGGGAACCCCCTTTATCGC
CGGCAAGAGGATCGTGCCTGTGATCGAGAACCACAGATTCACCGGCAGATAC
CGGGACCTGTACCCCGCCAACGAGCTGATTGCCCTGCTGGAAGAGAAGGGCA
TCGTGTTCCGGGACGGCAGCAACATCCTGCCCAAGCTGCTGGAAAATGACGA
CAGCCACGCCATCGATACCATGGTGGCACTGATCCGCAGCGTGCTGCAGATG
CGGAACAGCAATGCCGCCACCGGCGAGGACTACATCAATAGCCCAGTGCGG
GACCTGAACGGCGTGTCTTTCGACAGCAGATTCCAGAACCCCGAGTGGCCCA
TGGATGCCGACGCCAATGGCGCCTACCACATTGCCCTGAAGGGACAGCTGCT
GCTGAACCATCTGAAAGAGAGCAAAGACCTGAAACTGCAGAACGGCATCTC
CAACCAGGACTGGCTGGCCTATATCCAGGAACTGCGGAAC
C65S, C205S, C334S, C379S, C674S, and C1248S mutant Cas12a
DNA sequence
SEQ ID NO. 22
ATGACCCAGTTCGAGGGCTTCACCAACCTGTACCAGGTGTCCAAGACCCTGA
GATTCGAGCTGATCCCCCAGGGCAAGACACTGAAGCACATCCAGGAACAGG
GCTTCATCGAAGAGGACAAGGCCCGGAACGACCACTACAAAGAGCTGAAGC
CCATCATCTACCGGATCTACAAGACCTACGCCGACCAGTGCCTGCAGCTGGT
GCAGCTGGACTGGGAGAATCTGAGCGCCGCCATCGACAGCTACCGGAAAGA
GAAAACCGAGGAAACCCGGAACGCCCTGATCGAGGAACAGGCCACCTACAG
AAACGCCATCCACGACTACTTCATCGGCCGGACCGACAACCTGACCGACGCC
ATCAACAAGCGGCACGCCGAGATCTATAAGGGCCTGTTCAAGGCCGAGCTGT
TCAACGGCAAGGTGCTGAAGCAGCTGGGCACCGTGACCACCACCGAGCACG
AAAACGCCCTGCTGCGGAGCTTCGACAAGTTCACCACCTACTTCAGCGGCTT
CTACGAGAACCGGAAGAACGTGTTCAGCGCCGAGGACATCAGCACCGCCATC
CCCCACAGAATCGTGCAGGACAACTTCCCCAAGTTCAAAGAGAACTCTCACA
TCTTCACCCGGCTGATCACCGCCGTGCCCAGCCTGAGAGAACACTTCGAGAA
CGTGAAGAAGGCCATCGGCATCTTCGTGTCCACCAGCATCGAGGAAGTGTTC
AGCTTCCCATTCTACAACCAGCTGCTGACCCAGACCCAGATCGACCTGTATA
ATCAGCTGCTGGGCGGCATCAGCAGAGAGGCCGGCACCGAGAAGATCAAGG
GCCTGAACGAAGTGCTGAACCTGGCCATCCAGAAGAACGACGAGACAGCCC
ACATCATTGCCAGCCTGCCCCACCGGTTCATCCCTCTGTTCAAGCAGATCCTG
AGCGACAGAAACACCCTGAGCTTCATCCTGGAAGAGTTCAAGTCCGATGAGG
AAGTGATCCAGAGCTTCTCTAAGTATAAGACCCTGCTGAGGAACGAGAATGT
GCTGGAAACCGCCGAGGCCCTGTTCAATGAGCTGAACAGCATCGACCTGACC
CACATCTTTATCAGCCACAAGAAGCTGGAAACAATCAGCAGCGCCCTGTCTG
ACCACTGGGACACACTGCGGAATGCCCTGTACGAGCGGCGGATCTCTGAGCT
GACCGGCAAGATCACCAAGAGCGCCAAAGAAAAGGTGCAGCGGAGCCTGAA
GCACGAGGATATCAACCTGCAGGAAATCATCAGCGCCGCTGGCAAAGAACT
GAGCGAGGCCTTTAAGCAGAAAACCAGCGAGATCCTGTCCCACGCCCACGCC
GCACTGGATCAGCCTCTGCCTACCACCCTGAAGAAGCAGGAAGAGAAAGAG
ATCCTGAAGTCCCAGCTGGACAGCCTGCTGGGCCTGTACCATCTGCTGGATTG
GTTCGCCGTGGACGAGAGCAACGAGGTGGACCCCGAGTTCTCCGCCAGACTG
ACAGGCATCAAACTGGAAATGGAACCCAGCCTGTCCTTCTACAACAAGGCCA
GAAACTACGCCACCAAGAAACCCTACAGCGTGGAAAAGTTTAAGCTGAACTT
CCAGATGCCCACCCTGGCCAGCGGCTGGGACGTGAACAAAGAGAAGAACAA
CGGCGCCATCCTGTTCGTGAAGAACGGACTGTACTACCTGGGCATCATGCCT
AAGCAGAAGGGCAGATACAAGGCCCTGTCCTTTGAGCCCACCGAAAAGACC
AGCGAGGGCTTTGACAAGATGTACTACGATTACTTCCCCGACGCCGCCAAGA
TGATCCCCAAGTGCAGCACCCAGCTGAAGGCCGTGACCGCCCACTTTCAGAC
CCACACCACCCCCATCCTGCTGAGCAACAACTTCATCGAGCCCCTGGAAATC
ACCAAAGAGATCTACGACCTGAACAACCCCGAGAAAGAGCCCAAGAAGTTC
CAGACCGCCTACGCCAAGAAAACCGGCGACCAGAAGGGCTACCGCGAGGCT
CTGTCTAAGTGGATCGACTTTACCCGGGACTTCCTGAGCAAGTACACCAAGA
CCACCTCCATCGATCTGAGCAGCCTGCGGCCCAGCTCCCAGTACAAGGATCT
GGGCGAGTACTACGCCGAGCTGAACCCTCTGCTGTACCACATCAGCTTCCAG
CGGATCGCCGAAAAAGAAATCATGGACGCCGTGGAAACCGGCAAGCTGTAC
CTGTTCCAGATCTATAACAAGGACTTCGCCAAGGGCCACCACGGCAAGCCCA
ATCTGCACACCCTGTACTGGACCGGCCTGTTTAGCCCCGAGAATCTGGCCAA
GACCAGCATCAAGCTGAACGGCCAGGCCGAACTGTTTTACCGGCCCAAGAGC
CGGATGAAGCGGATGGCCCATAGACTGGGCGAGAAGATGCTGAACAAGAAA
CTGAAGGACCAGAAAACCCCTATCCCCGACACACTGTATCAGGAACTGTACG
ACTACGTGAACCACCGGCTGAGCCACGACCTGTCCGACGAAGCTAGAGCACT
GCTGCCCAACGTGATCACAAAAGAGGTGTCCCACGAGATCATCAAGGACCGG
CGGTTTACCTCCGATAAGTTCTTCTTCCACGTGCCCATCACCCTGAACTACCA
GGCCGCCAACAGCCCCAGCAAGTTCAACCAGAGAGTGAACGCCTACCTGAA
AGAGCACCCCGAGACACCCATCATTGGCATCGACAGAGGCGAGCGGAACCT
GATCTACATCACCGTGATCGACAGCACAGGCAAAATCCTGGAACAGAGAAG
CCTGAACACCATCCAGCAGTTCGACTACCAGAAGAAACTGGACAACCGGGA
AAAAGAACGGGTGGCCGCCAGACAGGCTTGGAGCGTCGTGGGCACCATTAA
GGACCTGAAGCAGGGCTACCTGAGCCAAGTGATTCACGAGATCGTGGACCTG
ATGATCCACTATCAGGCTGTGGTGGTGCTGGAAAACCTGAACTTCGGCTTCA
AGAGCAAGCGGACCGGAATCGCCGAGAAAGCCGTGTACCAGCAGTTTGAGA
AAATGCTGATCGACAAGCTGAATTGCCTGGTGCTGAAAGACTACCCCGCTGA
GAAAGTGGGAGGCGTGCTGAATCCCTACCAGCTGACCGACCAGTTCACCTCC
TTTGCCAAGATGGGAACCCAGAGCGGCTTCCTGTTCTACGTGCCAGCCCCCTA
CACCAGCAAGATCGACCCTCTGACCGGCTTCGTGGACCCCTTCGTGTGGAAA
ACCATCAAGAACCACGAGTCCCGGAAGCACTTCCTGGAAGGCTTTGACTTCC
TGCACTACGACGTGAAAACAGGCGATTTCATCCTGCACTTCAAGATGAATCG
GAATCTGTCCTTCCAGAGGGGCCTGCCCGGCTTCATGCCTGCCTGGGATATCG
TGTTCGAGAAGAATGAGACACAGTTCGACGCCAAGGGAACCCCCTTTATCGC
CGGCAAGAGGATCGTGCCTGTGATCGAGAACCACAGATTCACCGGCAGATAC
CGGGACCTGTACCCCGCCAACGAGCTGATTGCCCTGCTGGAAGAGAAGGGCA
TCGTGTTCCGGGACGGCAGCAACATCCTGCCCAAGCTGCTGGAAAATGACGA
CAGCCACGCCATCGATACCATGGTGGCACTGATCCGCAGCGTGCTGCAGATG
CGGAACAGCAATGCCGCCACCGGCGAGGACTACATCAATAGCCCAGTGCGG
GACCTGAACGGCGTGTCTTTCGACAGCAGATTCCAGAACCCCGAGTGGCCCA
TGGATGCCGACGCCAATGGCGCCTACCACATTGCCCTGAAGGGACAGCTGCT
GCTGAACCATCTGAAAGAGAGCAAAGACCTGAAACTGCAGAACGGCATCTC
CAACCAGGACTGGCTGGCCTATATCCAGGAACTGCGGAAC

### Example 2

### Novel Cas12a Substitution Mutants Enhance or Maintain the Cleavage Activity in a Bacterial-Based Activity Assay

The following example demonstrates that cysteine residues in Cas12a can be substituted with serine without negatively affecting enzymatic cleavage activity in a bacterial system. (Table 1). Bacterial strains that report Cas12a cleavage activity were transformed with a plasmid that expresses either wild-type Cas12a or mutant Cas12a containing individual cysteine to serine substitutions at the following positions: C65, C205, C334, C379, C608, C674, C1025, or C1248. The C65S, C205S, C334S, C379S, C674S, and C1248S substitutions demonstrated cleavage activity that was similar to wild-type Cas12a, which indicates that cysteine is not a critical amino acid at these positions. However, the single C608S and C1025S substitutions showed a decrease in cleavage activity indicating that these residues may be important at these positions, or that serine is not a tolerated change.

TABLE 1

| Cas12a expression | Bacterial Cleavage |
|---|---|
| plasmid | Activity (% survival) |
| Negative Control | <1% |
| Wild-Type | 68% |
| C65S | 65% |
| C205S | 80% |
| C334S | 78% |
| C379S | 62% |
| C608S | <1% |
| C674S | 88% |
| C1025S | 2% |
| C1248S | 62% |

### Table 1.

Novel Cas12a mutants in which cysteines were changed to serine at six out of eight positions increased the activity in a bacterial-based activity assay. The screening E. coli strains were transformed with Cas12a expression vectors (WT or plasmid that contained a change in one of the eight Cysteines present in Cas12a) and the crRNA targeting HPRT-38346 site on the toxin expression plasmid. The apparent activity of the different Cas12a plasmids can be predicted by the number of colonies that survived under arabinose selection when equal amount of plasmid is delivered. Increased survival rate was seen with mutations at amino acid positions C65, C205, C334, C379, C674, and C1248, while a decrease in the survival rate was seen when the change was made at positions C605 and C1025. This indicates that mutations at C65, C205, C334, C379, C674, and C1248 increase the cleavage activity of AsCas12a, but the single mutations at C605 and C1025 decrease the endonuclease activity of AsCas12a.

### Example 3

Novel Cas12a Substitution Mutants Enhance the Cleavage Activity in a Human Cell Line Based Activity Assay when Delivered as into Human Cells Via Plasmid Expression

The following example demonstrates the ability of Cas12a mutants to improve genome editing efficiency when delivered as a plasmid expression vector into human cells with lipid transfection.

Plasmids (0.5 μg) encoding wild-type or mutant Cas12a were transfected into HEK293 immortalized human cells using TransIT-X2 (Mirus Bio) lipid (0.5 μL per well). Two Cas12a mutants were tested. The first Cas12a mutant was a mutant in which four cysteine residues were substituted with serine. The first mutant contained substitutions at C205S, C379S, C674S, and C1248S. The second mutant was a mutant in which 6 cysteine residues were substituted with serine. The second mutant contained substitutions at C65S, C205S, C334S, C379S, C674S, and C1248S. The targeted protospacers and PAM sequences in HPRT loci (Seq ID No. 13-14) are shown in Table 2.

Experiments were performed in biological triplicate. After 48 hr at 37° C. with 5% CO₂, adherent cells were lysed with 0.05 ml QuickExtract™ DNA extraction solution. Cell lysates were incubated at 65° C. for 15 min followed by heat-inactivation at 98° C. for 3 min. Crude DNA samples were then diluted 3-fold and then an additional 5-fold with 0.1 ml ddH₂O and used as PCR templates.

PCR primers (Seq ID No. 15-16) are indicated in Table 2. PCR was used to amplify 1.1 kb fragments of the HPRT loci using the Q5 DNA Polymerase and the following cycling parameters: 98^{0:30}, (98^{0:10}, 65^{0:15}, 72^{0:60}) repeated 25 times, 72^{2:00}. Heteroduplexes were formed using the following cycling parameters: 95^{10:00} cooled to 85 over 1 min, 85^{1:00} cooled to 75 over 1 min, 75^{1:00} cooled to 65 over 1 min, 65^{1:00} cooled to 55 over 1 min, 55^{1:00} cooled to 45 over 1 min, 45^{1:00} cooled to 35^{1:00} over 1 min, 35^{1:00} cooled to 25 over 1 min, 25^{1:00}. Heteroduplexes were cleaved by the addition of 2 U T7 Endonuclease I (New England Biolabs) for 1 hr at 37 C, and cut products were analyzed by capillary electrophoresis (Fragment Analyzer, Advanced Analytical).

TABLE 2

| | | SEQ ID |
|---|---|---|
| Name | Sequence (5′-3′) | NO |
| HPRT-38330 | TAATTTCTACTCTTGTAGATGGTTAAAGA | SEQ ID |
| | TGGTTAAATGAT | No. 13 |
| HPRT-38228 | TAATTTCTACTCTTGTAGATTAATTAACA | SEQ ID |
| | GCTTGCTGGTGA | No. 14 |
| HPRT low GC | AAGAATGTTGTGATAAAAGGTGATGCT | SEQ ID |
| For | | No. 15 |
| HPRT low GC | ACACATCCATGGGACTTCTGCCTC | SEQ ID |
| Rev | | No. 16 |

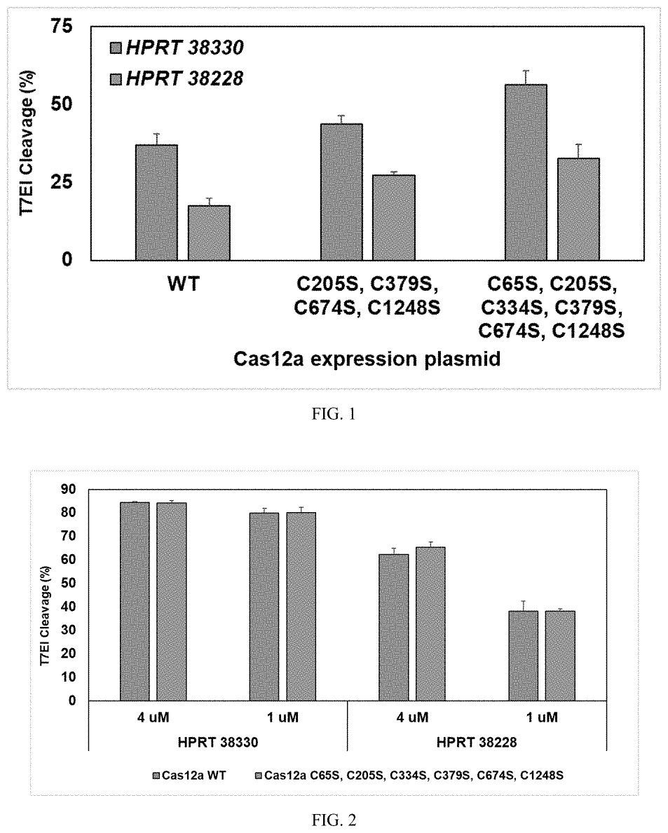

The endonuclease activity of wild type Cas12a and mutant Cas12a plasmids in human cells are described in FIG. 1. Plasmid delivery of a Cas12a mutant with four of eight cysteine residues changed to serine (C205S, C379S, C674S, and C1248S) resulted in increased cleavage activity (˜20%) as compared to the wild-type Cas12a plasmid. A further increase was seen (˜60%) when a Cas12a mutant containing six of eight cysteine to serine substitutions (C65S, C205S, C334S, C379S, C674S, and C1248S) was compared to the wild-type protein. Results from the bacterial cleavage system indicated that these substitutions were largely indistinguishable from wild-type Cas12a thereby making the finding that these substitutions collectively improve cleavage efficiency in human cells unexpected.

FIG. 1 demonstrates that Cas12a mutants with reduced cysteine content show increased editing efficiencies relative to wild-type Cas12a with plasmid-based expression in human cells. Editing efficiencies of Cas12a plasmid variants were examined in HEK293 Cells using a T7 endonuclease I assay (T7EI). Two crRNAs targeting the HPRT gene were each added together with a Cas12a plasmid (WT or one of the two variants) and delivered by Lipofection (TransIT-X2, Minis Bio). The genomic DNA was collected 48 hours following delivery to assess editing by T7EI.

### Example 4

Novel Cas12a Substitution Mutants Maintain the Cleavage Activity in a Human Cell Line Based Activity Assay when Delivered as into Human Cells Via Ribonucleoprotein (RNP)

The following example demonstrates that this invention increases genome editing efficiency when wild-type or mutant Cas12a is delivered into human cells as an RNP complex.

RNP complexes were formed (4 μM or 1 μM) with purified Cas12a proteins and Alt-R™ crRNAs (Seq ID No. 13-14) in Opti-MEM for 5 min at 25° C. The targeted protospacers and PAM sequences in HPRT loci are shown in Table 2. RNP complexes were then transfected into HEK293 cells by Lonza nucleofection. Experiments were performed in biological triplicate. After 48 hr at 37° C. with 5% CO₂, adherent cells were lysed with 0.05 ml QuickExtract™ DNA extraction solution. Cell lysates were incubated at 65° C. for 15 min followed by heat-inactivation at 98° C. for 3 min. Crude DNA samples were then diluted 3-fold and then an additional 5-fold with 0.1 ml ddH₂O and used as PCR templates. PCR primers (Seq ID No. 15-16) are indicated in Table 2. PCR was used to amplify 1.1 kb fragments of the HPRT loci using the Q5 DNA Polymerase and the following cycling parameters: 98^{0:30}, (98^{0:10}, 65^{0:15}, 72^{0:60}) repeated 25 times, 72^{2:00}. Heteroduplexes were formed using the following cycling parameters: 95^{10:00} cooled to 85 over 1 min, 85^{1:00} cooled to 75^{1:00} over 1 min, 75^{1:00} cooled to 65 over 1 min, 65^{1:00} cooled to 55 over 1 min, 55^{1:00} cooled to 45 over 1 min, 45^{1:00} cooled to 35 over 1 min, 35^{1:00} cooled to 25 over 1 min, 25^{1:00}. Heteroduplexes were cleaved by the addition of 2 U T7 Endonuclease I (New England Biolabs) for 1 hr at 37 C, and cut products were analyzed by capillary electrophoresis (Fragment Analyzer, Advanced Analytical).

Cleavage activity of WT AsCas12a protein was assessed in comparison to AsCas12a protein with the six cysteines changed to serine in FIG. 2. Comparable editing, as measured by T7E1 cleavage, was seen with the alternative Cas12a protein in comparison to the WT AsCas12a protein when delivered as an RNP at two different doses for two HPRT sites using electroporation. There is an increase in activity for the higher dose (4 uM) at the HPRT 38228 site where editing is often variable. This indicates that the mutant would be beneficial for sites where a lot of variability in editing is seen.

FIG. 2 demonstrates that Cas12a mutants with reduced cysteine content function similarly to wild-type Cas12a with RNP delivery into human cells. Editing efficiencies of Cas12a proteins were tested in HEK293 Cells using a T7 endonuclease I assay (T7EI). Cas12a crRNAs targeting the HPRT gene were used to form RNP complexes (1 μM and 4 μM) with WT or mutant (C65S, C205S, C334S, C379S, C674S, C1248S) Cas12a, which were delivered by electroporation into HEK293 cells. The genomic DNA was collected 48 hours following delivery to assess editing by T7EI

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but no limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

The term “wild-type Cas12a” (“wild-type enzyme” or “WT-Cas12a”) encompasses a protein having the identical amino acid sequence of the naturally-occurring Acidaminococcus sp. BV3L6 Cas12a (e.g., SEQ ID NO: 01) and that has biochemical and biological activity when combined with a suitable crRNA to form and active CRISPR/Cas12a endonuclease system.

The term “mutant Cas12a protein” encompasses protein forms having a different amino acid sequence form the wild-type Acidaminococcus sp. BV3L6 Cas12a and that have biochemical and biological activity when combined with a suitable guide RNA (for example sgRNA or dual crRNA:tracrRNA compositions) to form an active CRISPR-Cas12a endonuclease system. This includes orthologs and Cas12a variants having different amino acid sequences form the wild-type Acidaminococcus sp. BV3L6 Cas12a.

The term “polypeptide” refers to any linear or branched peptide comprising more than one amino acid. Polypeptide includes protein or fragment thereof or fusion thereof, provided such protein, fragment or fusion retains a useful biochemical or biological activity.

Fusion proteins typically include extra amino acid information that is not native to the protein to which the extra amino acid information is covalently attached. Such extra amino acid information may include tags that enable purification or identification of the fusion protein. Such extra amino acid information may include peptides that enable the fusion proteins to be transported into cells and/or transported to specific locations within cells. Examples of tags for these purposes include the following: AviTag, which is a peptide allowing biotinylation by the enzyme BirA so the protein can be isolated by streptavidin (GLNDIFEAQKIEWHE); Calmodulin-tag, which is a peptide bound by the protein calmodulin (KRRWKKNFIAVSAANRFKKISSSGAL); polyglutamate tag, which is a peptide binding efficiently to anion-exchange resin such as Mono-Q (EEEEEE); E-tag, which is a peptide recognized by an antibody (GAPVPYPDPLEPR); FLAG-tag, which is a peptide recognized by an antibody (DYKDDDDK); HA-tag, which is a peptide from hemagglutinin recognized by an antibody (YPYDVPDYA); His-tag, which is typically 5-10 histidines bound by a nickel or cobalt chelate (HHHHHH); Myc-tag, which is a peptide derived from c-myc recognized by an antibody (EQKLISEEDL); NE-tag, which is a novel 18-amino-acid synthetic peptide (TKENPRSNQEESYDDNES) recognized by a monoclonal IgG1 antibody, which is useful in a wide spectrum of applications including Western blotting, ELISA, flow cytometry, immunocytochemistry, immunoprecipitation, and affinity purification of recombinant proteins; S-tag, which is a peptide derived from Ribonuclease A (KETAAAKFERQHMDS); SBP-tag, which is a peptide which binds to streptavidin; (MDEKTTGWRGGHVVEGLAGELEQLRARLEHHPQGQREP); Softag 1, which is intended for mammalian expression (SLAELLNAGLGGS); Softag 3, which is intended for prokaryotic expression (TQDPSRVG); Strep-tag, which is a peptide which binds to streptavidin or the modified streptavidin called streptactin (Strep-tag II: WSHPQFEK); TC tag, which is a tetracysteine tag that is recognized by FlAsH and ReAsH biarsenical compounds (CCPGCC)V5 tag, which is a peptide recognized by an antibody (GKPIPNPLLGLDST); VSV-tag, a peptide recognized by an antibody (YTDIEMNRLGK); Xpress tag (DLYDDDDK); Isopeptag, which is a peptide which binds covalently to pilin-C protein (TDKDMTITFTNKKDAE); SpyTag, which is a peptide which binds covalently to SpyCatcher protein (AHIVMVDAYKPTK); SnoopTag, a peptide which binds covalently to SnoopCatcher protein (KLGDIEFIKVNK); BCCP (Biotin Carboxyl Carrier Protein), which is a protein domain biotinylated by BirA to enable recognition by streptavidin; Glutathione-S-transferase-tag, which is a protein that binds to immobilized glutathione; Green fluorescent protein-tag, which is a protein which is spontaneously fluorescent and can be bound by antibodies; HaloTag, which is a mutated bacterial haloalkane dehalogenase that covalently attaches to a reactive haloalkane substrate to allow attachment to a wide variety of substrates; Maltose binding protein-tag, a protein which binds to amylose agarose; Nustag; Thioredoxin-tag; and Fc-tag, derived from immunoglobulin Fc domain, which allows dimerization and solubilization and can be used for purification on Protein-A Sepharose. Nuclear localization signals (NLS), such as those obtained from SV40, allow for proteins to be transported to the nucleus immediately upon entering the cell. Given that the native Cas9 protein is bacterial in origin and therefore does not naturally comprise a NLS motif, addition of one or more NLS motifs to the recombinant Cas9 protein is expected to show improved genome editing activity when used in eukaryotic cells where the target genomic DNA substrate resides in the nucleus. One skilled in the art would appreciate these various fusion tag technologies, as well as how to make and use fusion proteins that include them.

What is claimed is:

1. An isolated mutant Cas12a comprising a substitution mutation selected from the group consisting of
a) a single substitution mutation introduced into the wild-type Cas12a protein selected from the following positions C65, C205, C334, C379, C608, C674, C1025, and C1248: or
b) a multiple substitution mutation introduced into the wild-type Cas12a protein selected from at least two of the following positions: C65, C205, C334, C379, C608, C674, C1025, and C1248.

2. The isolated mutant Cas12a protein of claim 1, wherein the isolated mutant Cas12a protein is selected form the group consisting of SEQ ID NO. 3, SEQ ID NO. 4, SEQ ID NO. 5, SEQ ID NO. 6, SEQ ID NO.7, SEQ ID NO. 8, SEQ ID NO. 9, SEQ ID NO. 10, SEQ ID NO. 11, and SEQ ID NO. 12.

3. The isolated mutant Cas12a protein of claim 1, wherein the isolated mutant Cas12 protein is selected from the group consisting of SEQ ID NO. 11 and SEQ ID NO. 12.

4. An isolated ribonucleoprotein complex, comprising:
a) the mutant Cas12a protein of claim 1; and
b) a gRNA complex,
wherein the isolated ribonucleoprotein complex is active as a CRISPR/Cas12a endonuclease system, wherein the resultant CRISPR/Cas12a endonuclease system displays maintained on-target editing activity relative to a wild-type CRISPR/Cas12a endonuclease system.

5. The isolated ribonucleoprotein complex of claim 4, wherein the mutant Cas12a protein is selected from the group consisting of SEQ ID NO. 3, SEQ ID NO. 4, SEQ ID NO. 5, SEQ ID NO. 6, SEQ ID NO.7, SEQ ID NO. 8, SEQ ID NO. 9, SEQ ID NO. 10, SEQ ID NO. 11, and SEQ ID NO. 12.

6. The CRISPR/Cas12a endonuclease system comprising a mutant Cas12a protein and a gRNA, wherein the CRISPR/Cas12a endonuclease system displays maintained on-target editing activity relative to a wild-type CRISPR/Cas12a endonuclease system.

7. The CRISPR/Cas12a endonuclease system of claim 6, wherein the CRISPR/Cas12a endonuclease system is encoded by a DNA expression vector.

8. The CRISPR/Cas12a endonuclease system of claim 7, wherein the DNA expression vector comprises a plasmid-borne vector.

9. The CRISPR/Cas12a endonuclease system of claim 8, wherein the DNA expression vector is selected form a bacterial expression vector and a eukaryotic expression vector.

10. An isolated nucleic acid encoding a mutant Cas12a protein, wherein the mutant Cas12a protein is active in CRISPR/Cas12a endonuclease system, wherein the CRISPR/Cas12a endonuclease system displays maintained on-target editing activity relative to a wild-type CRISPR/Cas12a endonuclease system.

11. The isolated nucleic acid encoding a mutant Cas12a protein of claim 10, wherein the mutant Cas12a protein comprises a substitution mutation selected from the group consisting of
a) a single substitution mutation introduced into the wild-type Cas12a protein selected from the following positions: C65, C205, C334, C379, C608, C674, C1025, and C1248; or
b) a multiple substitution mutation introduced into the wild-type Cas12a protein selected from at least two of the following positions: C65, C205, C334, C379, C608, C674, C1025, and C1248.

12. The isolated nucleic acid encoding a mutant Cas12a protein of claim 10, wherein the mutant Cas12a protein is selected from the group consisting of SEQ ID NO. 13, SEQ ID NO. 14, SEQ ID NO. 15, SEQ ID NO. 16, SEQ ID NO. 17, SEQ ID NO. 18, SEQ ID NO. 19, SEQ ID NO. 20, SEQ ID NO. 21, and SEQ ID NO. 22.


SEQUENCE LISTING (22 sequences)

SEQ ID NO: 1 - DNA, 3918 bp, Artificial Sequence
  Synthetic Polynucleotide
acccagttcgagggcttcaccaacctgtaccaggtgtccaagaccctgagattcgagctgatcccccagggcaagacactgaagcacatccaggaacagggcttcatcgaagaggacaaggcccggaacgaccactacaaagagctgaagcccatcatcgaccggatctacaagacctacgccgaccagtgcctgcagctggtgcagctggactgggagaatctgagcgccgccatcgacagctaccggaaagagaaaaccgaggaaacccggaacgccctgatcgaggaacaggccacctacagaaacgccatccacgactacttcatcggccggaccgacaacctgaccgacgccatcaacaagcggcacgccgagatctataagggcctgttcaaggccgagctgttcaacggcaaggtgctgaagcagctgggcaccgtgaccaccaccgagcacgaaaacgccctgctgcggagcttcgacaagttcaccacctacttcagcggcttctacgagaaccggaagaacgtgttcagcgccgaggacatcagcaccgccatcccccacagaatcgtgcaggacaacttccccaagttcaaagagaactgccacatcttcacccggctgatcaccgccgtgcccagcctgagagaacacttcgagaacgtgaagaaggccatcggcatcttcgtgtccaccagcatcgaggaagtgttcagcttcccattctacaaccagctgctgacccagacccagatcgacctgtataatcagctgctgggcggcatcagcagagaggccggcaccgagaagatcaagggcctgaacgaagtgctgaacctggccatccagaagaacgacgagacagcccacatcattgccagcctgccccaccggttcatccctctgttcaagcagatcctgagcgacagaaacaccctgagcttcatcctggaagagttcaagtccgatgaggaagtgatccagagcttctgcaagtataagaccctgctgaggaacgagaatgtgctggaaaccgccgaggccctgttcaatgagctgaacagcatcgacctgacccacatctttatcagccacaagaagctggaaacaatcagcagcgccctgtgcgaccactgggacacactgcggaatgccctgtacgagcggcggatctctgagctgaccggcaagatcaccaagagcgccaaagaaaaggtgcagcggagcctgaagcacgaggatatcaacctgcaggaaatcatcagcgccgctggcaaagaactgagcgaggcctttaagcagaaaaccagcgagatcctgtcccacgcccacgccgcactggatcagcctctgcctaccaccctgaagaagcaggaagagaaagagatcctgaagtcccagctggacagcctgctgggcctgtaccatctgctggattggttcgccgtggacgagagcaacgaggtggaccccgagttctccgccagactgacaggcatcaaactggaaatggaacccagcctgtccttctacaacaaggccagaaactacgccaccaagaaaccctacagcgtggaaaagtttaagctgaacttccagatgcccaccctggccagcggctgggacgtgaacaaagagaagaacaacggcgccatcctgttcgtgaagaacggactgtactacctgggcatcatgcctaagcagaagggcagatacaaggccctgtcctttgagcccaccgaaaagaccagcgagggctttgacaagatgtactacgattacttccccgacgccgccaagatgatccccaagtgcagcacccagctgaaggccgtgaccgcccactttcagacccacaccacccccatcctgctgagcaacaacttcatcgagcccctggaaatcaccaaagagatctacgacctgaacaaccccgagaaagagcccaagaagttccagaccgcctacgccaagaaaaccggcgaccagaagggctaccgcgaggctctgtgcaagtggatcgactttacccgggacttcctgagcaagtacaccaagaccacctccatcgatctgagcagcctgcggcccagctcccagtacaaggatctgggcgagtactacgccgagctgaaccctctgctgtaccacatcagcttccagcggatcgccgaaaaagaaatcatggacgccgtggaaaccggcaagctgtacctgttccagatctataacaaggacttcgccaagggccaccacggcaagcccaatctgcacaccctgtactggaccggcctgtttagccccgagaatctggccaagaccagcatcaagctgaacggccaggccgaactgttttaccggcccaagagccggatgaagcggatggcccatagactgggcgagaagatgctgaacaagaaactgaaggaccagaaaacccctatccccgacacactgtatcaggaactgtacgactacgtgaaccaccggctgagccacgacctgtccgacgaagctagagcactgctgcccaacgtgatcacaaaagaggtgtcccacgagatcatcaaggaccggcggtttacctccgataagttcttcttccacgtgcccatcaccctgaactaccaggccgccaacagccccagcaagttcaaccagagagtgaacgcctacctgaaagagcaccccgagacacccatcattggcatcgacagaggcgagcggaacctgatctacatcaccgtgatcgacagcacaggcaaaatcctggaacagagaagcctgaacaccatccagcagttcgactaccagaagaaactggacaaccgggaaaaagaacgggtggccgccagacaggcttggagcgtcgtgggcaccattaaggacctgaagcagggctacctgagccaagtgattcacgagatcgtggacctgatgatccactatcaggctgtggtggtgctggaaaacctgaacttcggcttcaagagcaagcggaccggaatcgccgagaaagccgtgtaccagcagtttgagaaaatgctgatcgacaagctgaattgcctggtgctgaaagactaccccgctgagaaagtgggaggcgtgctgaatccctaccagctgaccgaccagttcacctcctttgccaagatgggaacccagagcggcttcctgttctacgtgccagccccctacaccagcaagatcgaccctctgaccggcttcgtggaccccttcgtgtggaaaaccatcaagaaccacgagtcccggaagcacttcctggaaggctttgacttcctgcactacgacgtgaaaacaggcgatttcatcctgcacttcaagatgaatcggaatctgtccttccagaggggcctgcccggcttcatgcctgcctgggatatcgtgttcgagaagaatgagacacagttcgacgccaagggaaccccctttatcgccggcaagaggatcgtgcctgtgatcgagaaccacagattcaccggcagataccgggacctgtaccccgccaacgagctgattgccctgctggaagagaagggcatcgtgttccgggacggcagcaacatcctgcccaagctgctggaaaatgacgacagccacgccatcgataccatggtggcactgatccgcagcgtgctgcagatgcggaacagcaatgccgccaccggcgaggactacatcaatagcccagtgcgggacctgaacggcgtgtgcttcgacagcagattccagaaccccgagtggcccatggatgccgacgccaatggcgcctaccacattgccctgaagggacagctgctgctgaaccatctgaaagagagcaaagacctgaaactgcagaacggcatctccaaccaggactggctggcctatatccaggaactgcggaac

SEQ ID NO: 2 - protein, 1307 aa, Artificial Sequence
  Synthetic Polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 3 - protein, 1307 aa, Artificial Sequence
  Synthetic Polypeptide
MTQFEGFTNLYQVSKTLRFELIPQGKTLKHIQEQGFIEEDKARNDHYKELKPIIDRIYKTYADQSLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 4 - protein, 1307 aa, Artificial Sequence
  Synthetic Polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENSHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 5 - protein, 1307 aa, Artificial Sequence
  Synthetic Polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFSKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 6 - protein, 1307 aa, Artificial Sequence
  Synthetic Polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALSDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 7 - protein, 1307 aa, Artificial Sequence
  Synthetic Polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKSSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 8 - protein, 1307 aa, Artificial Sequence
  Synthetic Polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALSKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 9 - protein, 1307 aa, Artificial Sequence
  Synthetic Polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNSLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 10 - protein, 1307 aa, Artificial Sequence
  Synthetic Polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVSFDSRFQNPEWPMDADANGAYHIALKGQLLLNHLKESKDLKLQNGISNQDWLAYIQELRN

SEQ ID NO: 11 - protein, 1307 aa, Artificial Sequence
  Synthetic Polypeptide
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENSHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALSDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALSKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVSFDSRFQNPEWPMDADANGAYHIALKGQLLLNHLKESKDLKLQNGISNQDWLAYIQELRN

SEQ ID NO: 12 - protein, 1307 aa, Artificial Sequence
  Synthetic Polypeptide
MTQFEGFTNLYQVSKTLRFELIPQGKTLKHIQEQGFIEEDKARNDHYKELKPIIDRIYKTYADQSLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENSHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFSKYKTLLRNENVLETAEALFNELNSIDLTHIFISHKKLETISSALSDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALSKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVSFDSRFQNPEWPMDADANGAYHIALKGQLLLNHLKESKDLKLQNGISNQDWLAYIQELRN

SEQ ID NO: 13 - DNA, 3921 bp, Artificial Sequence
  Synthetic Polynucleotide
atgacccagttcgagggcttcaccaacctgtaccaggtgtccaagaccctgagattcgagctgatcccccagggcaagacactgaagcacatccaggaacagggcttcatcgaagaggacaaggcccggaacgaccactacaaagagctgaagcccatcatcgaccggatctacaagacctacgccgaccagtctctgcagctggtgcagctggactgggagaatctgagcgccgccatcgacagctaccggaaagagaaaaccgaggaaacccggaacgccctgatcgaggaacaggccacctacagaaacgccatccacgactacttcatcggccggaccgacaacctgaccgacgccatcaacaagcggcacgccgagatctataagggcctgttcaaggccgagctgttcaacggcaaggtgctgaagcagctgggcaccgtgaccaccaccgagcacgaaaacgccctgctgcggagcttcgacaagttcaccacctacttcagcggcttctacgagaaccggaagaacgtgttcagcgccgaggacatcagcaccgccatcccccacagaatcgtgcaggacaacttccccaagttcaaagagaactgccacatcttcacccggctgatcaccgccgtgcccagcctgagagaacacttcgagaacgtgaagaaggccatcggcatcttcgtgtccaccagcatcgaggaagtgttcagcttcccattctacaaccagctgctgacccagacccagatcgacctgtataatcagctgctgggcggcatcagcagagaggccggcaccgagaagatcaagggcctgaacgaagtgctgaacctggccatccagaagaacgacgagacagcccacatcattgccagcctgccccaccggttcatccctctgttcaagcagatcctgagcgacagaaacaccctgagcttcatcctggaagagttcaagtccgatgaggaagtgatccagagcttctgcaagtataagaccctgctgaggaacgagaatgtgctggaaaccgccgaggccctgttcaatgagctgaacagcatcgacctgacccacatctttatcagccacaagaagctggaaacaatcagcagcgccctgtgcgaccactgggacacactgcggaatgccctgtacgagcggcggatctctgagctgaccggcaagatcaccaagagcgccaaagaaaaggtgcagcggagcctgaagcacgaggatatcaacctgcaggaaatcatcagcgccgctggcaaagaactgagcgaggcctttaagcagaaaaccagcgagatcctgtcccacgcccacgccgcactggatcagcctctgcctaccaccctgaagaagcaggaagagaaagagatcctgaagtcccagctggacagcctgctgggcctgtaccatctgctggattggttcgccgtggacgagagcaacgaggtggaccccgagttctccgccagactgacaggcatcaaactggaaatggaacccagcctgtccttctacaacaaggccagaaactacgccaccaagaaaccctacagcgtggaaaagtttaagctgaacttccagatgcccaccctggccagcggctgggacgtgaacaaagagaagaacaacggcgccatcctgttcgtgaagaacggactgtactacctgggcatcatgcctaagcagaagggcagatacaaggccctgtcctttgagcccaccgaaaagaccagcgagggctttgacaagatgtactacgattacttccccgacgccgccaagatgatccccaagtgcagcacccagctgaaggccgtgaccgcccactttcagacccacaccacccccatcctgctgagcaacaacttcatcgagcccctggaaatcaccaaagagatctacgacctgaacaaccccgagaaagagcccaagaagttccagaccgcctacgccaagaaaaccggcgaccagaagggctaccgcgaggctctgtgcaagtggatcgactttacccgggacttcctgagcaagtacaccaagaccacctccatcgatctgagcagcctgcggcccagctcccagtacaaggatctgggcgagtactacgccgagctgaaccctctgctgtaccacatcagcttccagcggatcgccgaaaaagaaatcatggacgccgtggaaaccggcaagctgtacctgttccagatctataacaaggacttcgccaagggccaccacggcaagcccaatctgcacaccctgtactggaccggcctgtttagccccgagaatctggccaagaccagcatcaagctgaacggccaggccgaactgttttaccggcccaagagccggatgaagcggatggcccatagactgggcgagaagatgctgaacaagaaactgaaggaccagaaaacccctatccccgacacactgtatcaggaactgtacgactacgtgaaccaccggctgagccacgacctgtccgacgaagctagagcactgctgcccaacgtgatcacaaaagaggtgtcccacgagatcatcaaggaccggcggtttacctccgataagttcttcttccacgtgcccatcaccctgaactaccaggccgccaacagccccagcaagttcaaccagagagtgaacgcctacctgaaagagcaccccgagacacccatcattggcatcgacagaggcgagcggaacctgatctacatcaccgtgatcgacagcacaggcaaaatcctggaacagagaagcctgaacaccatccagcagttcgactaccagaagaaactggacaaccgggaaaaagaacgggtggccgccagacaggcttggagcgtcgtgggcaccattaaggacctgaagcagggctacctgagccaagtgattcacgagatcgtggacctgatgatccactatcaggctgtggtggtgctggaaaacctgaacttcggcttcaagagcaagcggaccggaatcgccgagaaagccgtgtaccagcagtttgagaaaatgctgatcgacaagctgaattgcctggtgctgaaagactaccccgctgagaaagtgggaggcgtgctgaatccctaccagctgaccgaccagttcacctcctttgccaagatgggaacccagagcggcttcctgttctacgtgccagccccctacaccagcaagatcgaccctctgaccggcttcgtggaccccttcgtgtggaaaaccatcaagaaccacgagtcccggaagcacttcctggaaggctttgacttcctgcactacgacgtgaaaacaggcgatttcatcctgcacttcaagatgaatcggaatctgtccttccagaggggcctgcccggcttcatgcctgcctgggatatcgtgttcgagaagaatgagacacagttcgacgccaagggaaccccctttatcgccggcaagaggatcgtgcctgtgatcgagaaccacagattcaccggcagataccgggacctgtaccccgccaacgagctgattgccctgctggaagagaagggcatcgtgttccgggacggcagcaacatcctgcccaagctgctggaaaatgacgacagccacgccatcgataccatggtggcactgatccgcagcgtgctgcagatgcggaacagcaatgccgccaccggcgaggactacatcaatagcccagtgcgggacctgaacggcgtgtgcttcgacagcagattccagaaccccgagtggcccatggatgccgacgccaatggcgcctaccacattgccctgaagggacagctgctgctgaaccatctgaaagagagcaaagacctgaaactgcagaacggcatctccaaccaggactggctggcctatatccaggaactgcggaac

SEQ ID NO: 14 - DNA, 3921 bp, Artificial Sequence
  Synthetic Polynucleotide
atgacccagttcgagggcttcaccaacctgtaccaggtgtccaagaccctgagattcgagctgatcccccagggcaagacactgaagcacatccaggaacagggcttcatcgaagaggacaaggcccggaacgaccactacaaagagctgaagcccatcatcgaccggatctacaagacctacgccgaccagtgcctgcagctggtgcagctggactgggagaatctgagcgccgccatcgacagctaccggaaagagaaaaccgaggaaacccggaacgccctgatcgaggaacaggccacctacagaaacgccatccacgactacttcatcggccggaccgacaacctgaccgacgccatcaacaagcggcacgccgagatctataagggcctgttcaaggccgagctgttcaacggcaaggtgctgaagcagctgggcaccgtgaccaccaccgagcacgaaaacgccctgctgcggagcttcgacaagttcaccacctacttcagcggcttctacgagaaccggaagaacgtgttcagcgccgaggacatcagcaccgccatcccccacagaatcgtgcaggacaacttccccaagttcaaagagaactctcacatcttcacccggctgatcaccgccgtgcccagcctgagagaacacttcgagaacgtgaagaaggccatcggcatcttcgtgtccaccagcatcgaggaagtgttcagcttcccattctacaaccagctgctgacccagacccagatcgacctgtataatcagctgctgggcggcatcagcagagaggccggcaccgagaagatcaagggcctgaacgaagtgctgaacctggccatccagaagaacgacgagacagcccacatcattgccagcctgccccaccggttcatccctctgttcaagcagatcctgagcgacagaaacaccctgagcttcatcctggaagagttcaagtccgatgaggaagtgatccagagcttctgcaagtataagaccctgctgaggaacgagaatgtgctggaaaccgccgaggccctgttcaatgagctgaacagcatcgacctgacccacatctttatcagccacaagaagctggaaacaatcagcagcgccctgtgcgaccactgggacacactgcggaatgccctgtacgagcggcggatctctgagctgaccggcaagatcaccaagagcgccaaagaaaaggtgcagcggagcctgaagcacgaggatatcaacctgcaggaaatcatcagcgccgctggcaaagaactgagcgaggcctttaagcagaaaaccagcgagatcctgtcccacgcccacgccgcactggatcagcctctgcctaccaccctgaagaagcaggaagagaaagagatcctgaagtcccagctggacagcctgctgggcctgtaccatctgctggattggttcgccgtggacgagagcaacgaggtggaccccgagttctccgccagactgacaggcatcaaactggaaatggaacccagcctgtccttctacaacaaggccagaaactacgccaccaagaaaccctacagcgtggaaaagtttaagctgaacttccagatgcccaccctggccagcggctgggacgtgaacaaagagaagaacaacggcgccatcctgttcgtgaagaacggactgtactacctgggcatcatgcctaagcagaagggcagatacaaggccctgtcctttgagcccaccgaaaagaccagcgagggctttgacaagatgtactacgattacttccccgacgccgccaagatgatccccaagtgcagcacccagctgaaggccgtgaccgcccactttcagacccacaccacccccatcctgctgagcaacaacttcatcgagcccctggaaatcaccaaagagatctacgacctgaacaaccccgagaaagagcccaagaagttccagaccgcctacgccaagaaaaccggcgaccagaagggctaccgcgaggctctgtgcaagtggatcgactttacccgggacttcctgagcaagtacaccaagaccacctccatcgatctgagcagcctgcggcccagctcccagtacaaggatctgggcgagtactacgccgagctgaaccctctgctgtaccacatcagcttccagcggatcgccgaaaaagaaatcatggacgccgtggaaaccggcaagctgtacctgttccagatctataacaaggacttcgccaagggccaccacggcaagcccaatctgcacaccctgtactggaccggcctgtttagccccgagaatctggccaagaccagcatcaagctgaacggccaggccgaactgttttaccggcccaagagccggatgaagcggatggcccatagactgggcgagaagatgctgaacaagaaactgaaggaccagaaaacccctatccccgacacactgtatcaggaactgtacgactacgtgaaccaccggctgagccacgacctgtccgacgaagctagagcactgctgcccaacgtgatcacaaaagaggtgtcccacgagatcatcaaggaccggcggtttacctccgataagttcttcttccacgtgcccatcaccctgaactaccaggccgccaacagccccagcaagttcaaccagagagtgaacgcctacctgaaagagcaccccgagacacccatcattggcatcgacagaggcgagcggaacctgatctacatcaccgtgatcgacagcacaggcaaaatcctggaacagagaagcctgaacaccatccagcagttcgactaccagaagaaactggacaaccgggaaaaagaacgggtggccgccagacaggcttggagcgtcgtgggcaccattaaggacctgaagcagggctacctgagccaagtgattcacgagatcgtggacctgatgatccactatcaggctgtggtggtgctggaaaacctgaacttcggcttcaagagcaagcggaccggaatcgccgagaaagccgtgtaccagcagtttgagaaaatgctgatcgacaagctgaattgcctggtgctgaaagactaccccgctgagaaagtgggaggcgtgctgaatccctaccagctgaccgaccagttcacctcctttgccaagatgggaacccagagcggcttcctgttctacgtgccagccccctacaccagcaagatcgaccctctgaccggcttcgtggaccccttcgtgtggaaaaccatcaagaaccacgagtcccggaagcacttcctggaaggctttgacttcctgcactacgacgtgaaaacaggcgatttcatcctgcacttcaagatgaatcggaatctgtccttccagaggggcctgcccggcttcatgcctgcctgggatatcgtgttcgagaagaatgagacacagttcgacgccaagggaaccccctttatcgccggcaagaggatcgtgcctgtgatcgagaaccacagattcaccggcagataccgggacctgtaccccgccaacgagctgattgccctgctggaagagaagggcatcgtgttccgggacggcagcaacatcctgcccaagctgctggaaaatgacgacagccacgccatcgataccatggtggcactgatccgcagcgtgctgcagatgcggaacagcaatgccgccaccggcgaggactacatcaatagcccagtgcgggacctgaacggcgtgtgcttcgacagcagattccagaaccccgagtggcccatggatgccgacgccaatggcgcctaccacattgccctgaagggacagctgctgctgaaccatctgaaagagagcaaagacctgaaactgcagaacggcatctccaaccaggactggctggcctatatccaggaactgcggaac

SEQ ID NO: 15 - DNA, 3921 bp, Artificial Sequence
  Synthetic Polynucleotide
atgacccagttcgagggcttcaccaacctgtaccaggtgtccaagaccctgagattcgagctgatcccccagggcaagacactgaagcacatccaggaacagggcttcatcgaagaggacaaggcccggaacgaccactacaaagagctgaagcccatcatcgaccggatctacaagacctacgccgaccagtgcctgcagctggtgcagctggactgggagaatctgagcgccgccatcgacagctaccggaaagagaaaaccgaggaaacccggaacgccctgatcgaggaacaggccacctacagaaacgccatccacgactacttcatcggccggaccgacaacctgaccgacgccatcaacaagcggcacgccgagatctataagggcctgttcaaggccgagctgttcaacggcaaggtgctgaagcagctgggcaccgtgaccaccaccgagcacgaaaacgccctgctgcggagcttcgacaagttcaccacctacttcagcggcttctacgagaaccggaagaacgtgttcagcgccgaggacatcagcaccgccatcccccacagaatcgtgcaggacaacttccccaagttcaaagagaactgccacatcttcacccggctgatcaccgccgtgcccagcctgagagaacacttcgagaacgtgaagaaggccatcggcatcttcgtgtccaccagcatcgaggaagtgttcagcttcccattctacaaccagctgctgacccagacccagatcgacctgtataatcagctgctgggcggcatcagcagagaggccggcaccgagaagatcaagggcctgaacgaagtgctgaacctggccatccagaagaacgacgagacagcccacatcattgccagcctgccccaccggttcatccctctgttcaagcagatcctgagcgacagaaacaccctgagcttcatcctggaagagttcaagtccgatgaggaagtgatccagagcttctctaagtataagaccctgctgaggaacgagaatgtgctggaaaccgccgaggccctgttcaatgagctgaacagcatcgacctgacccacatctttatcagccacaagaagctggaaacaatcagcagcgccctgtgcgaccactgggacacactgcggaatgccctgtacgagcggcggatctctgagctgaccggcaagatcaccaagagcgccaaagaaaaggtgcagcggagcctgaagcacgaggatatcaacctgcaggaaatcatcagcgccgctggcaaagaactgagcgaggcctttaagcagaaaaccagcgagatcctgtcccacgcccacgccgcactggatcagcctctgcctaccaccctgaagaagcaggaagagaaagagatcctgaagtcccagctggacagcctgctgggcctgtaccatctgctggattggttcgccgtggacgagagcaacgaggtggaccccgagttctccgccagactgacaggcatcaaactggaaatggaacccagcctgtccttctacaacaaggccagaaactacgccaccaagaaaccctacagcgtggaaaagtttaagctgaacttccagatgcccaccctggccagcggctgggacgtgaacaaagagaagaacaacggcgccatcctgttcgtgaagaacggactgtactacctgggcatcatgcctaagcagaagggcagatacaaggccctgtcctttgagcccaccgaaaagaccagcgagggctttgacaagatgtactacgattacttccccgacgccgccaagatgatccccaagtgcagcacccagctgaaggccgtgaccgcccactttcagacccacaccacccccatcctgctgagcaacaacttcatcgagcccctggaaatcaccaaagagatctacgacctgaacaaccccgagaaagagcccaagaagttccagaccgcctacgccaagaaaaccggcgaccagaagggctaccgcgaggctctgtgcaagtggatcgactttacccgggacttcctgagcaagtacaccaagaccacctccatcgatctgagcagcctgcggcccagctcccagtacaaggatctgggcgagtactacgccgagctgaaccctctgctgtaccacatcagcttccagcggatcgccgaaaaagaaatcatggacgccgtggaaaccggcaagctgtacctgttccagatctataacaaggacttcgccaagggccaccacggcaagcccaatctgcacaccctgtactggaccggcctgtttagccccgagaatctggccaagaccagcatcaagctgaacggccaggccgaactgttttaccggcccaagagccggatgaagcggatggcccatagactgggcgagaagatgctgaacaagaaactgaaggaccagaaaacccctatccccgacacactgtatcaggaactgtacgactacgtgaaccaccggctgagccacgacctgtccgacgaagctagagcactgctgcccaacgtgatcacaaaagaggtgtcccacgagatcatcaaggaccggcggtttacctccgataagttcttcttccacgtgcccatcaccctgaactaccaggccgccaacagccccagcaagttcaaccagagagtgaacgcctacctgaaagagcaccccgagacacccatcattggcatcgacagaggcgagcggaacctgatctacatcaccgtgatcgacagcacaggcaaaatcctggaacagagaagcctgaacaccatccagcagttcgactaccagaagaaactggacaaccgggaaaaagaacgggtggccgccagacaggcttggagcgtcgtgggcaccattaaggacctgaagcagggctacctgagccaagtgattcacgagatcgtggacctgatgatccactatcaggctgtggtggtgctggaaaacctgaacttcggcttcaagagcaagcggaccggaatcgccgagaaagccgtgtaccagcagtttgagaaaatgctgatcgacaagctgaattgcctggtgctgaaagactaccccgctgagaaagtgggaggcgtgctgaatccctaccagctgaccgaccagttcacctcctttgccaagatgggaacccagagcggcttcctgttctacgtgccagccccctacaccagcaagatcgaccctctgaccggcttcgtggaccccttcgtgtggaaaaccatcaagaaccacgagtcccggaagcacttcctggaaggctttgacttcctgcactacgacgtgaaaacaggcgatttcatcctgcacttcaagatgaatcggaatctgtccttccagaggggcctgcccggcttcatgcctgcctgggatatcgtgttcgagaagaatgagacacagttcgacgccaagggaaccccctttatcgccggcaagaggatcgtgcctgtgatcgagaaccacagattcaccggcagataccgggacctgtaccccgccaacgagctgattgccctgctggaagagaagggcatcgtgttccgggacggcagcaacatcctgcccaagctgctggaaaatgacgacagccacgccatcgataccatggtggcactgatccgcagcgtgctgcagatgcggaacagcaatgccgccaccggcgaggactacatcaatagcccagtgcgggacctgaacggcgtgtgcttcgacagcagattccagaaccccgagtggcccatggatgccgacgccaatggcgcctaccacattgccctgaagggacagctgctgctgaaccatctgaaagagagcaaagacctgaaactgcagaacggcatctccaaccaggactggctggcctatatccaggaactgcggaac

SEQ ID NO: 16 - DNA, 3921 bp, Artificial Sequence
  Synthetic Polynucleotide
atgacccagttcgagggcttcaccaacctgtaccaggtgtccaagaccctgagattcgagctgatcccccagggcaagacactgaagcacatccaggaacagggcttcatcgaagaggacaaggcccggaacgaccactacaaagagctgaagcccatcatcgaccggatctacaagacctacgccgaccagtgcctgcagctggtgcagctggactgggagaatctgagcgccgccatcgacagctaccggaaagagaaaaccgaggaaacccggaacgccctgatcgaggaacaggccacctacagaaacgccatccacgactacttcatcggccggaccgacaacctgaccgacgccatcaacaagcggcacgccgagatctataagggcctgttcaaggccgagctgttcaacggcaaggtgctgaagcagctgggcaccgtgaccaccaccgagcacgaaaacgccctgctgcggagcttcgacaagttcaccacctacttcagcggcttctacgagaaccggaagaacgtgttcagcgccgaggacatcagcaccgccatcccccacagaatcgtgcaggacaacttccccaagttcaaagagaactgccacatcttcacccggctgatcaccgccgtgcccagcctgagagaacacttcgagaacgtgaagaaggccatcggcatcttcgtgtccaccagcatcgaggaagtgttcagcttcccattctacaaccagctgctgacccagacccagatcgacctgtataatcagctgctgggcggcatcagcagagaggccggcaccgagaagatcaagggcctgaacgaagtgctgaacctggccatccagaagaacgacgagacagcccacatcattgccagcctgccccaccggttcatccctctgttcaagcagatcctgagcgacagaaacaccctgagcttcatcctggaagagttcaagtccgatgaggaagtgatccagagcttctgcaagtataagaccctgctgaggaacgagaatgtgctggaaaccgccgaggccctgttcaatgagctgaacagcatcgacctgacccacatctttatcagccacaagaagctggaaacaatcagcagcgccctgtctgaccactgggacacactgcggaatgccctgtacgagcggcggatctctgagctgaccggcaagatcaccaagagcgccaaagaaaaggtgcagcggagcctgaagcacgaggatatcaacctgcaggaaatcatcagcgccgctggcaaagaactgagcgaggcctttaagcagaaaaccagcgagatcctgtcccacgcccacgccgcactggatcagcctctgcctaccaccctgaagaagcaggaagagaaagagatcctgaagtcccagctggacagcctgctgggcctgtaccatctgctggattggttcgccgtggacgagagcaacgaggtggaccccgagttctccgccagactgacaggcatcaaactggaaatggaacccagcctgtccttctacaacaaggccagaaactacgccaccaagaaaccctacagcgtggaaaagtttaagctgaacttccagatgcccaccctggccagcggctgggacgtgaacaaagagaagaacaacggcgccatcctgttcgtgaagaacggactgtactacctgggcatcatgcctaagcagaagggcagatacaaggccctgtcctttgagcccaccgaaaagaccagcgagggctttgacaagatgtactacgattacttccccgacgccgccaagatgatccccaagtgcagcacccagctgaaggccgtgaccgcccactttcagacccacaccacccccatcctgctgagcaacaacttcatcgagcccctggaaatcaccaaagagatctacgacctgaacaaccccgagaaagagcccaagaagttccagaccgcctacgccaagaaaaccggcgaccagaagggctaccgcgaggctctgtgcaagtggatcgactttacccgggacttcctgagcaagtacaccaagaccacctccatcgatctgagcagcctgcggcccagctcccagtacaaggatctgggcgagtactacgccgagctgaaccctctgctgtaccacatcagcttccagcggatcgccgaaaaagaaatcatggacgccgtggaaaccggcaagctgtacctgttccagatctataacaaggacttcgccaagggccaccacggcaagcccaatctgcacaccctgtactggaccggcctgtttagccccgagaatctggccaagaccagcatcaagctgaacggccaggccgaactgttttaccggcccaagagccggatgaagcggatggcccatagactgggcgagaagatgctgaacaagaaactgaaggaccagaaaacccctatccccgacacactgtatcaggaactgtacgactacgtgaaccaccggctgagccacgacctgtccgacgaagctagagcactgctgcccaacgtgatcacaaaagaggtgtcccacgagatcatcaaggaccggcggtttacctccgataagttcttcttccacgtgcccatcaccctgaactaccaggccgccaacagccccagcaagttcaaccagagagtgaacgcctacctgaaagagcaccccgagacacccatcattggcatcgacagaggcgagcggaacctgatctacatcaccgtgatcgacagcacaggcaaaatcctggaacagagaagcctgaacaccatccagcagttcgactaccagaagaaactggacaaccgggaaaaagaacgggtggccgccagacaggcttggagcgtcgtgggcaccattaaggacctgaagcagggctacctgagccaagtgattcacgagatcgtggacctgatgatccactatcaggctgtggtggtgctggaaaacctgaacttcggcttcaagagcaagcggaccggaatcgccgagaaagccgtgtaccagcagtttgagaaaatgctgatcgacaagctgaattgcctggtgctgaaagactaccccgctgagaaagtgggaggcgtgctgaatccctaccagctgaccgaccagttcacctcctttgccaagatgggaacccagagcggcttcctgttctacgtgccagccccctacaccagcaagatcgaccctctgaccggcttcgtggaccccttcgtgtggaaaaccatcaagaaccacgagtcccggaagcacttcctggaaggctttgacttcctgcactacgacgtgaaaacaggcgatttcatcctgcacttcaagatgaatcggaatctgtccttccagaggggcctgcccggcttcatgcctgcctgggatatcgtgttcgagaagaatgagacacagttcgacgccaagggaaccccctttatcgccggcaagaggatcgtgcctgtgatcgagaaccacagattcaccggcagataccgggacctgtaccccgccaacgagctgattgccctgctggaagagaagggcatcgtgttccgggacggcagcaacatcctgcccaagctgctggaaaatgacgacagccacgccatcgataccatggtggcactgatccgcagcgtgctgcagatgcggaacagcaatgccgccaccggcgaggactacatcaatagcccagtgcgggacctgaacggcgtgtgcttcgacagcagattccagaaccccgagtggcccatggatgccgacgccaatggcgcctaccacattgccctgaagggacagctgctgctgaaccatctgaaagagagcaaagacctgaaactgcagaacggcatctccaaccaggactggctggcctatatccaggaactgcggaac

SEQ ID NO: 17 - DNA, 3921 bp, Artificial Sequence
  Synthetic Polynucleotide
atgacccagttcgagggcttcaccaacctgtaccaggtgtccaagaccctgagattcgagctgatcccccagggcaagacactgaagcacatccaggaacagggcttcatcgaagaggacaaggcccggaacgaccactacaaagagctgaagcccatcatcgaccggatctacaagacctacgccgaccagtgcctgcagctggtgcagctggactgggagaatctgagcgccgccatcgacagctaccggaaagagaaaaccgaggaaacccggaacgccctgatcgaggaacaggccacctacagaaacgccatccacgactacttcatcggccggaccgacaacctgaccgacgccatcaacaagcggcacgccgagatctataagggcctgttcaaggccgagctgttcaacggcaaggtgctgaagcagctgggcaccgtgaccaccaccgagcacgaaaacgccctgctgcggagcttcgacaagttcaccacctacttcagcggcttctacgagaaccggaagaacgtgttcagcgccgaggacatcagcaccgccatcccccacagaatcgtgcaggacaacttccccaagttcaaagagaactgccacatcttcacccggctgatcaccgccgtgcccagcctgagagaacacttcgagaacgtgaagaaggccatcggcatcttcgtgtccaccagcatcgaggaagtgttcagcttcccattctacaaccagctgctgacccagacccagatcgacctgtataatcagctgctgggcggcatcagcagagaggccggcaccgagaagatcaagggcctgaacgaagtgctgaacctggccatccagaagaacgacgagacagcccacatcattgccagcctgccccaccggttcatccctctgttcaagcagatcctgagcgacagaaacaccctgagcttcatcctggaagagttcaagtccgatgaggaagtgatccagagcttctgcaagtataagaccctgctgaggaacgagaatgtgctggaaaccgccgaggccctgttcaatgagctgaacagcatcgacctgacccacatctttatcagccacaagaagctggaaacaatcagcagcgccctgtgcgaccactgggacacactgcggaatgccctgtacgagcggcggatctctgagctgaccggcaagatcaccaagagcgccaaagaaaaggtgcagcggagcctgaagcacgaggatatcaacctgcaggaaatcatcagcgccgctggcaaagaactgagcgaggcctttaagcagaaaaccagcgagatcctgtcccacgcccacgccgcactggatcagcctctgcctaccaccctgaagaagcaggaagagaaagagatcctgaagtcccagctggacagcctgctgggcctgtaccatctgctggattggttcgccgtggacgagagcaacgaggtggaccccgagttctccgccagactgacaggcatcaaactggaaatggaacccagcctgtccttctacaacaaggccagaaactacgccaccaagaaaccctacagcgtggaaaagtttaagctgaacttccagatgcccaccctggccagcggctgggacgtgaacaaagagaagaacaacggcgccatcctgttcgtgaagaacggactgtactacctgggcatcatgcctaagcagaagggcagatacaaggccctgtcctttgagcccaccgaaaagaccagcgagggctttgacaagatgtactacgattacttccccgacgccgccaagatgatccccaagtctagcacccagctgaaggccgtgaccgcccactttcagacccacaccacccccatcctgctgagcaacaacttcatcgagcccctggaaatcaccaaagagatctacgacctgaacaaccccgagaaagagcccaagaagttccagaccgcctacgccaagaaaaccggcgaccagaagggctaccgcgaggctctgtgcaagtggatcgactttacccgggacttcctgagcaagtacaccaagaccacctccatcgatctgagcagcctgcggcccagctcccagtacaaggatctgggcgagtactacgccgagctgaaccctctgctgtaccacatcagcttccagcggatcgccgaaaaagaaatcatggacgccgtggaaaccggcaagctgtacctgttccagatctataacaaggacttcgccaagggccaccacggcaagcccaatctgcacaccctgtactggaccggcctgtttagccccgagaatctggccaagaccagcatcaagctgaacggccaggccgaactgttttaccggcccaagagccggatgaagcggatggcccatagactgggcgagaagatgctgaacaagaaactgaaggaccagaaaacccctatccccgacacactgtatcaggaactgtacgactacgtgaaccaccggctgagccacgacctgtccgacgaagctagagcactgctgcccaacgtgatcacaaaagaggtgtcccacgagatcatcaaggaccggcggtttacctccgataagttcttcttccacgtgcccatcaccctgaactaccaggccgccaacagccccagcaagttcaaccagagagtgaacgcctacctgaaagagcaccccgagacacccatcattggcatcgacagaggcgagcggaacctgatctacatcaccgtgatcgacagcacaggcaaaatcctggaacagagaagcctgaacaccatccagcagttcgactaccagaagaaactggacaaccgggaaaaagaacgggtggccgccagacaggcttggagcgtcgtgggcaccattaaggacctgaagcagggctacctgagccaagtgattcacgagatcgtggacctgatgatccactatcaggctgtggtggtgctggaaaacctgaacttcggcttcaagagcaagcggaccggaatcgccgagaaagccgtgtaccagcagtttgagaaaatgctgatcgacaagctgaattgcctggtgctgaaagactaccccgctgagaaagtgggaggcgtgctgaatccctaccagctgaccgaccagttcacctcctttgccaagatgggaacccagagcggcttcctgttctacgtgccagccccctacaccagcaagatcgaccctctgaccggcttcgtggaccccttcgtgtggaaaaccatcaagaaccacgagtcccggaagcacttcctggaaggctttgacttcctgcactacgacgtgaaaacaggcgatttcatcctgcacttcaagatgaatcggaatctgtccttccagaggggcctgcccggcttcatgcctgcctgggatatcgtgttcgagaagaatgagacacagttcgacgccaagggaaccccctttatcgccggcaagaggatcgtgcctgtgatcgagaaccacagattcaccggcagataccgggacctgtaccccgccaacgagctgattgccctgctggaagagaagggcatcgtgttccgggacggcagcaacatcctgcccaagctgctggaaaatgacgacagccacgccatcgataccatggtggcactgatccgcagcgtgctgcagatgcggaacagcaatgccgccaccggcgaggactacatcaatagcccagtgcgggacctgaacggcgtgtgcttcgacagcagattccagaaccccgagtggcccatggatgccgacgccaatggcgcctaccacattgccctgaagggacagctgctgctgaaccatctgaaagagagcaaagacctgaaactgcagaacggcatctccaaccaggactggctggcctatatccaggaactgcggaac

SEQ ID NO: 18 - DNA, 3921 bp, Artificial Sequence
  Synthetic Polynucleotide
atgacccagttcgagggcttcaccaacctgtaccaggtgtccaagaccctgagattcgagctgatcccccagggcaagacactgaagcacatccaggaacagggcttcatcgaagaggacaaggcccggaacgaccactacaaagagctgaagcccatcatcgaccggatctacaagacctacgccgaccagtgcctgcagctggtgcagctggactgggagaatctgagcgccgccatcgacagctaccggaaagagaaaaccgaggaaacccggaacgccctgatcgaggaacaggccacctacagaaacgccatccacgactacttcatcggccggaccgacaacctgaccgacgccatcaacaagcggcacgccgagatctataagggcctgttcaaggccgagctgttcaacggcaaggtgctgaagcagctgggcaccgtgaccaccaccgagcacgaaaacgccctgctgcggagcttcgacaagttcaccacctacttcagcggcttctacgagaaccggaagaacgtgttcagcgccgaggacatcagcaccgccatcccccacagaatcgtgcaggacaacttccccaagttcaaagagaactgccacatcttcacccggctgatcaccgccgtgcccagcctgagagaacacttcgagaacgtgaagaaggccatcggcatcttcgtgtccaccagcatcgaggaagtgttcagcttcccattctacaaccagctgctgacccagacccagatcgacctgtataatcagctgctgggcggcatcagcagagaggccggcaccgagaagatcaagggcctgaacgaagtgctgaacctggccatccagaagaacgacgagacagcccacatcattgccagcctgccccaccggttcatccctctgttcaagcagatcctgagcgacagaaacaccctgagcttcatcctggaagagttcaagtccgatgaggaagtgatccagagcttctgcaagtataagaccctgctgaggaacgagaatgtgctggaaaccgccgaggccctgttcaatgagctgaacagcatcgacctgacccacatctttatcagccacaagaagctggaaacaatcagcagcgccctgtgcgaccactgggacacactgcggaatgccctgtacgagcggcggatctctgagctgaccggcaagatcaccaagagcgccaaagaaaaggtgcagcggagcctgaagcacgaggatatcaacctgcaggaaatcatcagcgccgctggcaaagaactgagcgaggcctttaagcagaaaaccagcgagatcctgtcccacgcccacgccgcactggatcagcctctgcctaccaccctgaagaagcaggaagagaaagagatcctgaagtcccagctggacagcctgctgggcctgtaccatctgctggattggttcgccgtggacgagagcaacgaggtggaccccgagttctccgccagactgacaggcatcaaactggaaatggaacccagcctgtccttctacaacaaggccagaaactacgccaccaagaaaccctacagcgtggaaaagtttaagctgaacttccagatgcccaccctggccagcggctgggacgtgaacaaagagaagaacaacggcgccatcctgttcgtgaagaacggactgtactacctgggcatcatgcctaagcagaagggcagatacaaggccctgtcctttgagcccaccgaaaagaccagcgagggctttgacaagatgtactacgattacttccccgacgccgccaagatgatccccaagtgcagcacccagctgaaggccgtgaccgcccactttcagacccacaccacccccatcctgctgagcaacaacttcatcgagcccctggaaatcaccaaagagatctacgacctgaacaaccccgagaaagagcccaagaagttccagaccgcctacgccaagaaaaccggcgaccagaagggctaccgcgaggctctgtctaagtggatcgactttacccgggacttcctgagcaagtacaccaagaccacctccatcgatctgagcagcctgcggcccagctcccagtacaaggatctgggcgagtactacgccgagctgaaccctctgctgtaccacatcagcttccagcggatcgccgaaaaagaaatcatggacgccgtggaaaccggcaagctgtacctgttccagatctataacaaggacttcgccaagggccaccacggcaagcccaatctgcacaccctgtactggaccggcctgtttagccccgagaatctggccaagaccagcatcaagctgaacggccaggccgaactgttttaccggcccaagagccggatgaagcggatggcccatagactgggcgagaagatgctgaacaagaaactgaaggaccagaaaacccctatccccgacacactgtatcaggaactgtacgactacgtgaaccaccggctgagccacgacctgtccgacgaagctagagcactgctgcccaacgtgatcacaaaagaggtgtcccacgagatcatcaaggaccggcggtttacctccgataagttcttcttccacgtgcccatcaccctgaactaccaggccgccaacagccccagcaagttcaaccagagagtgaacgcctacctgaaagagcaccccgagacacccatcattggcatcgacagaggcgagcggaacctgatctacatcaccgtgatcgacagcacaggcaaaatcctggaacagagaagcctgaacaccatccagcagttcgactaccagaagaaactggacaaccgggaaaaagaacgggtggccgccagacaggcttggagcgtcgtgggcaccattaaggacctgaagcagggctacctgagccaagtgattcacgagatcgtggacctgatgatccactatcaggctgtggtggtgctggaaaacctgaacttcggcttcaagagcaagcggaccggaatcgccgagaaagccgtgtaccagcagtttgagaaaatgctgatcgacaagctgaattgcctggtgctgaaagactaccccgctgagaaagtgggaggcgtgctgaatccctaccagctgaccgaccagttcacctcctttgccaagatgggaacccagagcggcttcctgttctacgtgccagccccctacaccagcaagatcgaccctctgaccggcttcgtggaccccttcgtgtggaaaaccatcaagaaccacgagtcccggaagcacttcctggaaggctttgacttcctgcactacgacgtgaaaacaggcgatttcatcctgcacttcaagatgaatcggaatctgtccttccagaggggcctgcccggcttcatgcctgcctgggatatcgtgttcgagaagaatgagacacagttcgacgccaagggaaccccctttatcgccggcaagaggatcgtgcctgtgatcgagaaccacagattcaccggcagataccgggacctgtaccccgccaacgagctgattgccctgctggaagagaagggcatcgtgttccgggacggcagcaacatcctgcccaagctgctggaaaatgacgacagccacgccatcgataccatggtggcactgatccgcagcgtgctgcagatgcggaacagcaatgccgccaccggcgaggactacatcaatagcccagtgcgggacctgaacggcgtgtgcttcgacagcagattccagaaccccgagtggcccatggatgccgacgccaatggcgcctaccacattgccctgaagggacagctgctgctgaaccatctgaaagagagcaaagacctgaaactgcagaacggcatctccaaccaggactggctggcctatatccaggaactgcggaac

SEQ ID NO: 19 - DNA, 3921 bp, Artificial Sequence
  Synthetic Polynucleotide
atgacccagttcgagggcttcaccaacctgtaccaggtgtccaagaccctgagattcgagctgatcccccagggcaagacactgaagcacatccaggaacagggcttcatcgaagaggacaaggcccggaacgaccactacaaagagctgaagcccatcatcgaccggatctacaagacctacgccgaccagtgcctgcagctggtgcagctggactgggagaatctgagcgccgccatcgacagctaccggaaagagaaaaccgaggaaacccggaacgccctgatcgaggaacaggccacctacagaaacgccatccacgactacttcatcggccggaccgacaacctgaccgacgccatcaacaagcggcacgccgagatctataagggcctgttcaaggccgagctgttcaacggcaaggtgctgaagcagctgggcaccgtgaccaccaccgagcacgaaaacgccctgctgcggagcttcgacaagttcaccacctacttcagcggcttctacgagaaccggaagaacgtgttcagcgccgaggacatcagcaccgccatcccccacagaatcgtgcaggacaacttccccaagttcaaagagaactgccacatcttcacccggctgatcaccgccgtgcccagcctgagagaacacttcgagaacgtgaagaaggccatcggcatcttcgtgtccaccagcatcgaggaagtgttcagcttcccattctacaaccagctgctgacccagacccagatcgacctgtataatcagctgctgggcggcatcagcagagaggccggcaccgagaagatcaagggcctgaacgaagtgctgaacctggccatccagaagaacgacgagacagcccacatcattgccagcctgccccaccggttcatccctctgttcaagcagatcctgagcgacagaaacaccctgagcttcatcctggaagagttcaagtccgatgaggaagtgatccagagcttctgcaagtataagaccctgctgaggaacgagaatgtgctggaaaccgccgaggccctgttcaatgagctgaacagcatcgacctgacccacatctttatcagccacaagaagctggaaacaatcagcagcgccctgtgcgaccactgggacacactgcggaatgccctgtacgagcggcggatctctgagctgaccggcaagatcaccaagagcgccaaagaaaaggtgcagcggagcctgaagcacgaggatatcaacctgcaggaaatcatcagcgccgctggcaaagaactgagcgaggcctttaagcagaaaaccagcgagatcctgtcccacgcccacgccgcactggatcagcctctgcctaccaccctgaagaagcaggaagagaaagagatcctgaagtcccagctggacagcctgctgggcctgtaccatctgctggattggttcgccgtggacgagagcaacgaggtggaccccgagttctccgccagactgacaggcatcaaactggaaatggaacccagcctgtccttctacaacaaggccagaaactacgccaccaagaaaccctacagcgtggaaaagtttaagctgaacttccagatgcccaccctggccagcggctgggacgtgaacaaagagaagaacaacggcgccatcctgttcgtgaagaacggactgtactacctgggcatcatgcctaagcagaagggcagatacaaggccctgtcctttgagcccaccgaaaagaccagcgagggctttgacaagatgtactacgattacttccccgacgccgccaagatgatccccaagtgcagcacccagctgaaggccgtgaccgcccactttcagacccacaccacccccatcctgctgagcaacaacttcatcgagcccctggaaatcaccaaagagatctacgacctgaacaaccccgagaaagagcccaagaagttccagaccgcctacgccaagaaaaccggcgaccagaagggctaccgcgaggctctgtgcaagtggatcgactttacccgggacttcctgagcaagtacaccaagaccacctccatcgatctgagcagcctgcggcccagctcccagtacaaggatctgggcgagtactacgccgagctgaaccctctgctgtaccacatcagcttccagcggatcgccgaaaaagaaatcatggacgccgtggaaaccggcaagctgtacctgttccagatctataacaaggacttcgccaagggccaccacggcaagcccaatctgcacaccctgtactggaccggcctgtttagccccgagaatctggccaagaccagcatcaagctgaacggccaggccgaactgttttaccggcccaagagccggatgaagcggatggcccatagactgggcgagaagatgctgaacaagaaactgaaggaccagaaaacccctatccccgacacactgtatcaggaactgtacgactacgtgaaccaccggctgagccacgacctgtccgacgaagctagagcactgctgcccaacgtgatcacaaaagaggtgtcccacgagatcatcaaggaccggcggtttacctccgataagttcttcttccacgtgcccatcaccctgaactaccaggccgccaacagccccagcaagttcaaccagagagtgaacgcctacctgaaagagcaccccgagacacccatcattggcatcgacagaggcgagcggaacctgatctacatcaccgtgatcgacagcacaggcaaaatcctggaacagagaagcctgaacaccatccagcagttcgactaccagaagaaactggacaaccgggaaaaagaacgggtggccgccagacaggcttggagcgtcgtgggcaccattaaggacctgaagcagggctacctgagccaagtgattcacgagatcgtggacctgatgatccactatcaggctgtggtggtgctggaaaacctgaacttcggcttcaagagcaagcggaccggaatcgccgagaaagccgtgtaccagcagtttgagaaaatgctgatcgacaagctgaattctctggtgctgaaagactaccccgctgagaaagtgggaggcgtgctgaatccctaccagctgaccgaccagttcacctcctttgccaagatgggaacccagagcggcttcctgttctacgtgccagccccctacaccagcaagatcgaccctctgaccggcttcgtggaccccttcgtgtggaaaaccatcaagaaccacgagtcccggaagcacttcctggaaggctttgacttcctgcactacgacgtgaaaacaggcgatttcatcctgcacttcaagatgaatcggaatctgtccttccagaggggcctgcccggcttcatgcctgcctgggatatcgtgttcgagaagaatgagacacagttcgacgccaagggaaccccctttatcgccggcaagaggatcgtgcctgtgatcgagaaccacagattcaccggcagataccgggacctgtaccccgccaacgagctgattgccctgctggaagagaagggcatcgtgttccgggacggcagcaacatcctgcccaagctgctggaaaatgacgacagccacgccatcgataccatggtggcactgatccgcagcgtgctgcagatgcggaacagcaatgccgccaccggcgaggactacatcaatagcccagtgcgggacctgaacggcgtgtgcttcgacagcagattccagaaccccgagtggcccatggatgccgacgccaatggcgcctaccacattgccctgaagggacagctgctgctgaaccatctgaaagagagcaaagacctgaaactgcagaacggcatctccaaccaggactggctggcctatatccaggaactgcggaac

SEQ ID NO: 20 - DNA, 3921 bp, Artificial Sequence
  Synthetic Polynucleotide
atgacccagttcgagggcttcaccaacctgtaccaggtgtccaagaccctgagattcgagctgatcccccagggcaagacactgaagcacatccaggaacagggcttcatcgaagaggacaaggcccggaacgaccactacaaagagctgaagcccatcatcgaccggatctacaagacctacgccgaccagtgcctgcagctggtgcagctggactgggagaatctgagcgccgccatcgacagctaccggaaagagaaaaccgaggaaacccggaacgccctgatcgaggaacaggccacctacagaaacgccatccacgactacttcatcggccggaccgacaacctgaccgacgccatcaacaagcggcacgccgagatctataagggcctgttcaaggccgagctgttcaacggcaaggtgctgaagcagctgggcaccgtgaccaccaccgagcacgaaaacgccctgctgcggagcttcgacaagttcaccacctacttcagcggcttctacgagaaccggaagaacgtgttcagcgccgaggacatcagcaccgccatcccccacagaatcgtgcaggacaacttccccaagttcaaagagaactgccacatcttcacccggctgatcaccgccgtgcccagcctgagagaacacttcgagaacgtgaagaaggccatcggcatcttcgtgtccaccagcatcgaggaagtgttcagcttcccattctacaaccagctgctgacccagacccagatcgacctgtataatcagctgctgggcggcatcagcagagaggccggcaccgagaagatcaagggcctgaacgaagtgctgaacctggccatccagaagaacgacgagacagcccacatcattgccagcctgccccaccggttcatccctctgttcaagcagatcctgagcgacagaaacaccctgagcttcatcctggaagagttcaagtccgatgaggaagtgatccagagcttctgcaagtataagaccctgctgaggaacgagaatgtgctggaaaccgccgaggccctgttcaatgagctgaacagcatcgacctgacccacatctttatcagccacaagaagctggaaacaatcagcagcgccctgtgcgaccactgggacacactgcggaatgccctgtacgagcggcggatctctgagctgaccggcaagatcaccaagagcgccaaagaaaaggtgcagcggagcctgaagcacgaggatatcaacctgcaggaaatcatcagcgccgctggcaaagaactgagcgaggcctttaagcagaaaaccagcgagatcctgtcccacgcccacgccgcactggatcagcctctgcctaccaccctgaagaagcaggaagagaaagagatcctgaagtcccagctggacagcctgctgggcctgtaccatctgctggattggttcgccgtggacgagagcaacgaggtggaccccgagttctccgccagactgacaggcatcaaactggaaatggaacccagcctgtccttctacaacaaggccagaaactacgccaccaagaaaccctacagcgtggaaaagtttaagctgaacttccagatgcccaccctggccagcggctgggacgtgaacaaagagaagaacaacggcgccatcctgttcgtgaagaacggactgtactacctgggcatcatgcctaagcagaagggcagatacaaggccctgtcctttgagcccaccgaaaagaccagcgagggctttgacaagatgtactacgattacttccccgacgccgccaagatgatccccaagtgcagcacccagctgaaggccgtgaccgcccactttcagacccacaccacccccatcctgctgagcaacaacttcatcgagcccctggaaatcaccaaagagatctacgacctgaacaaccccgagaaagagcccaagaagttccagaccgcctacgccaagaaaaccggcgaccagaagggctaccgcgaggctctgtgcaagtggatcgactttacccgggacttcctgagcaagtacaccaagaccacctccatcgatctgagcagcctgcggcccagctcccagtacaaggatctgggcgagtactacgccgagctgaaccctctgctgtaccacatcagcttccagcggatcgccgaaaaagaaatcatggacgccgtggaaaccggcaagctgtacctgttccagatctataacaaggacttcgccaagggccaccacggcaagcccaatctgcacaccctgtactggaccggcctgtttagccccgagaatctggccaagaccagcatcaagctgaacggccaggccgaactgttttaccggcccaagagccggatgaagcggatggcccatagactgggcgagaagatgctgaacaagaaactgaaggaccagaaaacccctatccccgacacactgtatcaggaactgtacgactacgtgaaccaccggctgagccacgacctgtccgacgaagctagagcactgctgcccaacgtgatcacaaaagaggtgtcccacgagatcatcaaggaccggcggtttacctccgataagttcttcttccacgtgcccatcaccctgaactaccaggccgccaacagccccagcaagttcaaccagagagtgaacgcctacctgaaagagcaccccgagacacccatcattggcatcgacagaggcgagcggaacctgatctacatcaccgtgatcgacagcacaggcaaaatcctggaacagagaagcctgaacaccatccagcagttcgactaccagaagaaactggacaaccgggaaaaagaacgggtggccgccagacaggcttggagcgtcgtgggcaccattaaggacctgaagcagggctacctgagccaagtgattcacgagatcgtggacctgatgatccactatcaggctgtggtggtgctggaaaacctgaacttcggcttcaagagcaagcggaccggaatcgccgagaaagccgtgtaccagcagtttgagaaaatgctgatcgacaagctgaattgcctggtgctgaaagactaccccgctgagaaagtgggaggcgtgctgaatccctaccagctgaccgaccagttcacctcctttgccaagatgggaacccagagcggcttcctgttctacgtgccagccccctacaccagcaagatcgaccctctgaccggcttcgtggaccccttcgtgtggaaaaccatcaagaaccacgagtcccggaagcacttcctggaaggctttgacttcctgcactacgacgtgaaaacaggcgatttcatcctgcacttcaagatgaatcggaatctgtccttccagaggggcctgcccggcttcatgcctgcctgggatatcgtgttcgagaagaatgagacacagttcgacgccaagggaaccccctttatcgccggcaagaggatcgtgcctgtgatcgagaaccacagattcaccggcagataccgggacctgtaccccgccaacgagctgattgccctgctggaagagaagggcatcgtgttccgggacggcagcaacatcctgcccaagctgctggaaaatgacgacagccacgccatcgataccatggtggcactgatccgcagcgtgctgcagatgcggaacagcaatgccgccaccggcgaggactacatcaatagcccagtgcgggacctgaacggcgtgtctttcgacagcagattccagaaccccgagtggcccatggatgccgacgccaatggcgcctaccacattgccctgaagggacagctgctgctgaaccatctgaaagagagcaaagacctgaaactgcagaacggcatctccaaccaggactggctggcctatatccaggaactgcggaac

SEQ ID NO: 21 - DNA, 3921 bp, Artificial Sequence
  Synthetic Polynucleotide
atgacccagttcgagggcttcaccaacctgtaccaggtgtccaagaccctgagattcgagctgatcccccagggcaagacactgaagcacatccaggaacagggcttcatcgaagaggacaaggcccggaacgaccactacaaagagctgaagcccatcatcgaccggatctacaagacctacgccgaccagtgcctgcagctggtgcagctggactgggagaatctgagcgccgccatcgacagctaccggaaagagaaaaccgaggaaacccggaacgccctgatcgaggaacaggccacctacagaaacgccatccacgactacttcatcggccggaccgacaacctgaccgacgccatcaacaagcggcacgccgagatctataagggcctgttcaaggccgagctgttcaacggcaaggtgctgaagcagctgggcaccgtgaccaccaccgagcacgaaaacgccctgctgcggagcttcgacaagttcaccacctacttcagcggcttctacgagaaccggaagaacgtgttcagcgccgaggacatcagcaccgccatcccccacagaatcgtgcaggacaacttccccaagttcaaagagaactctcacatcttcacccggctgatcaccgccgtgcccagcctgagagaacacttcgagaacgtgaagaaggccatcggcatcttcgtgtccaccagcatcgaggaagtgttcagcttcccattctacaaccagctgctgacccagacccagatcgacctgtataatcagctgctgggcggcatcagcagagaggccggcaccgagaagatcaagggcctgaacgaagtgctgaacctggccatccagaagaacgacgagacagcccacatcattgccagcctgccccaccggttcatccctctgttcaagcagatcctgagcgacagaaacaccctgagcttcatcctggaagagttcaagtccgatgaggaagtgatccagagcttctgcaagtataagaccctgctgaggaacgagaatgtgctggaaaccgccgaggccctgttcaatgagctgaacagcatcgacctgacccacatctttatcagccacaagaagctggaaacaatcagcagcgccctgtctgaccactgggacacactgcggaatgccctgtacgagcggcggatctctgagctgaccggcaagatcaccaagagcgccaaagaaaaggtgcagcggagcctgaagcacgaggatatcaacctgcaggaaatcatcagcgccgctggcaaagaactgagcgaggcctttaagcagaaaaccagcgagatcctgtcccacgcccacgccgcactggatcagcctctgcctaccaccctgaagaagcaggaagagaaagagatcctgaagtcccagctggacagcctgctgggcctgtaccatctgctggattggttcgccgtggacgagagcaacgaggtggaccccgagttctccgccagactgacaggcatcaaactggaaatggaacccagcctgtccttctacaacaaggccagaaactacgccaccaagaaaccctacagcgtggaaaagtttaagctgaacttccagatgcccaccctggccagcggctgggacgtgaacaaagagaagaacaacggcgccatcctgttcgtgaagaacggactgtactacctgggcatcatgcctaagcagaagggcagatacaaggccctgtcctttgagcccaccgaaaagaccagcgagggctttgacaagatgtactacgattacttccccgacgccgccaagatgatccccaagtgcagcacccagctgaaggccgtgaccgcccactttcagacccacaccacccccatcctgctgagcaacaacttcatcgagcccctggaaatcaccaaagagatctacgacctgaacaaccccgagaaagagcccaagaagttccagaccgcctacgccaagaaaaccggcgaccagaagggctaccgcgaggctctgtctaagtggatcgactttacccgggacttcctgagcaagtacaccaagaccacctccatcgatctgagcagcctgcggcccagctcccagtacaaggatctgggcgagtactacgccgagctgaaccctctgctgtaccacatcagcttccagcggatcgccgaaaaagaaatcatggacgccgtggaaaccggcaagctgtacctgttccagatctataacaaggacttcgccaagggccaccacggcaagcccaatctgcacaccctgtactggaccggcctgtttagccccgagaatctggccaagaccagcatcaagctgaacggccaggccgaactgttttaccggcccaagagccggatgaagcggatggcccatagactgggcgagaagatgctgaacaagaaactgaaggaccagaaaacccctatccccgacacactgtatcaggaactgtacgactacgtgaaccaccggctgagccacgacctgtccgacgaagctagagcactgctgcccaacgtgatcacaaaagaggtgtcccacgagatcatcaaggaccggcggtttacctccgataagttcttcttccacgtgcccatcaccctgaactaccaggccgccaacagccccagcaagttcaaccagagagtgaacgcctacctgaaagagcaccccgagacacccatcattggcatcgacagaggcgagcggaacctgatctacatcaccgtgatcgacagcacaggcaaaatcctggaacagagaagcctgaacaccatccagcagttcgactaccagaagaaactggacaaccgggaaaaagaacgggtggccgccagacaggcttggagcgtcgtgggcaccattaaggacctgaagcagggctacctgagccaagtgattcacgagatcgtggacctgatgatccactatcaggctgtggtggtgctggaaaacctgaacttcggcttcaagagcaagcggaccggaatcgccgagaaagccgtgtaccagcagtttgagaaaatgctgatcgacaagctgaattgcctggtgctgaaagactaccccgctgagaaagtgggaggcgtgctgaatccctaccagctgaccgaccagttcacctcctttgccaagatgggaacccagagcggcttcctgttctacgtgccagccccctacaccagcaagatcgaccctctgaccggcttcgtggaccccttcgtgtggaaaaccatcaagaaccacgagtcccggaagcacttcctggaaggctttgacttcctgcactacgacgtgaaaacaggcgatttcatcctgcacttcaagatgaatcggaatctgtccttccagaggggcctgcccggcttcatgcctgcctgggatatcgtgttcgagaagaatgagacacagttcgacgccaagggaaccccctttatcgccggcaagaggatcgtgcctgtgatcgagaaccacagattcaccggcagataccgggacctgtaccccgccaacgagctgattgccctgctggaagagaagggcatcgtgttccgggacggcagcaacatcctgcccaagctgctggaaaatgacgacagccacgccatcgataccatggtggcactgatccgcagcgtgctgcagatgcggaacagcaatgccgccaccggcgaggactacatcaatagcccagtgcgggacctgaacggcgtgtctttcgacagcagattccagaaccccgagtggcccatggatgccgacgccaatggcgcctaccacattgccctgaagggacagctgctgctgaaccatctgaaagagagcaaagacctgaaactgcagaacggcatctccaaccaggactggctggcctatatccaggaactgcggaac

SEQ ID NO: 22 - DNA, 3921 bp, Artificial Sequence
  Synthetic Polynucleotide
atgacccagttcgagggcttcaccaacctgtaccaggtgtccaagaccctgagattcgagctgatcccccagggcaagacactgaagcacatccaggaacagggcttcatcgaagaggacaaggcccggaacgaccactacaaagagctgaagcccatcatctaccggatctacaagacctacgccgaccagtgcctgcagctggtgcagctggactgggagaatctgagcgccgccatcgacagctaccggaaagagaaaaccgaggaaacccggaacgccctgatcgaggaacaggccacctacagaaacgccatccacgactacttcatcggccggaccgacaacctgaccgacgccatcaacaagcggcacgccgagatctataagggcctgttcaaggccgagctgttcaacggcaaggtgctgaagcagctgggcaccgtgaccaccaccgagcacgaaaacgccctgctgcggagcttcgacaagttcaccacctacttcagcggcttctacgagaaccggaagaacgtgttcagcgccgaggacatcagcaccgccatcccccacagaatcgtgcaggacaacttccccaagttcaaagagaactctcacatcttcacccggctgatcaccgccgtgcccagcctgagagaacacttcgagaacgtgaagaaggccatcggcatcttcgtgtccaccagcatcgaggaagtgttcagcttcccattctacaaccagctgctgacccagacccagatcgacctgtataatcagctgctgggcggcatcagcagagaggccggcaccgagaagatcaagggcctgaacgaagtgctgaacctggccatccagaagaacgacgagacagcccacatcattgccagcctgccccaccggttcatccctctgttcaagcagatcctgagcgacagaaacaccctgagcttcatcctggaagagttcaagtccgatgaggaagtgatccagagcttctctaagtataagaccctgctgaggaacgagaatgtgctggaaaccgccgaggccctgttcaatgagctgaacagcatcgacctgacccacatctttatcagccacaagaagctggaaacaatcagcagcgccctgtctgaccactgggacacactgcggaatgccctgtacgagcggcggatctctgagctgaccggcaagatcaccaagagcgccaaagaaaaggtgcagcggagcctgaagcacgaggatatcaacctgcaggaaatcatcagcgccgctggcaaagaactgagcgaggcctttaagcagaaaaccagcgagatcctgtcccacgcccacgccgcactggatcagcctctgcctaccaccctgaagaagcaggaagagaaagagatcctgaagtcccagctggacagcctgctgggcctgtaccatctgctggattggttcgccgtggacgagagcaacgaggtggaccccgagttctccgccagactgacaggcatcaaactggaaatggaacccagcctgtccttctacaacaaggccagaaactacgccaccaagaaaccctacagcgtggaaaagtttaagctgaacttccagatgcccaccctggccagcggctgggacgtgaacaaagagaagaacaacggcgccatcctgttcgtgaagaacggactgtactacctgggcatcatgcctaagcagaagggcagatacaaggccctgtcctttgagcccaccgaaaagaccagcgagggctttgacaagatgtactacgattacttccccgacgccgccaagatgatccccaagtgcagcacccagctgaaggccgtgaccgcccactttcagacccacaccacccccatcctgctgagcaacaacttcatcgagcccctggaaatcaccaaagagatctacgacctgaacaaccccgagaaagagcccaagaagttccagaccgcctacgccaagaaaaccggcgaccagaagggctaccgcgaggctctgtctaagtggatcgactttacccgggacttcctgagcaagtacaccaagaccacctccatcgatctgagcagcctgcggcccagctcccagtacaaggatctgggcgagtactacgccgagctgaaccctctgctgtaccacatcagcttccagcggatcgccgaaaaagaaatcatggacgccgtggaaaccggcaagctgtacctgttccagatctataacaaggacttcgccaagggccaccacggcaagcccaatctgcacaccctgtactggaccggcctgtttagccccgagaatctggccaagaccagcatcaagctgaacggccaggccgaactgttttaccggcccaagagccggatgaagcggatggcccatagactgggcgagaagatgctgaacaagaaactgaaggaccagaaaacccctatccccgacacactgtatcaggaactgtacgactacgtgaaccaccggctgagccacgacctgtccgacgaagctagagcactgctgcccaacgtgatcacaaaagaggtgtcccacgagatcatcaaggaccggcggtttacctccgataagttcttcttccacgtgcccatcaccctgaactaccaggccgccaacagccccagcaagttcaaccagagagtgaacgcctacctgaaagagcaccccgagacacccatcattggcatcgacagaggcgagcggaacctgatctacatcaccgtgatcgacagcacaggcaaaatcctggaacagagaagcctgaacaccatccagcagttcgactaccagaagaaactggacaaccgggaaaaagaacgggtggccgccagacaggcttggagcgtcgtgggcaccattaaggacctgaagcagggctacctgagccaagtgattcacgagatcgtggacctgatgatccactatcaggctgtggtggtgctggaaaacctgaacttcggcttcaagagcaagcggaccggaatcgccgagaaagccgtgtaccagcagtttgagaaaatgctgatcgacaagctgaattgcctggtgctgaaagactaccccgctgagaaagtgggaggcgtgctgaatccctaccagctgaccgaccagttcacctcctttgccaagatgggaacccagagcggcttcctgttctacgtgccagccccctacaccagcaagatcgaccctctgaccggcttcgtggaccccttcgtgtggaaaaccatcaagaaccacgagtcccggaagcacttcctggaaggctttgacttcctgcactacgacgtgaaaacaggcgatttcatcctgcacttcaagatgaatcggaatctgtccttccagaggggcctgcccggcttcatgcctgcctgggatatcgtgttcgagaagaatgagacacagttcgacgccaagggaaccccctttatcgccggcaagaggatcgtgcctgtgatcgagaaccacagattcaccggcagataccgggacctgtaccccgccaacgagctgattgccctgctggaagagaagggcatcgtgttccgggacggcagcaacatcctgcccaagctgctggaaaatgacgacagccacgccatcgataccatggtggcactgatccgcagcgtgctgcagatgcggaacagcaatgccgccaccggcgaggactacatcaatagcccagtgcgggacctgaacggcgtgtctttcgacagcagattccagaaccccgagtggcccatggatgccgacgccaatggcgcctaccacattgccctgaagggacagctgctgctgaaccatctgaaagagagcaaagacctgaaactgcagaacggcatctccaaccaggactggctggcctatatccaggaactgcggaac